(19) United States
(12) Patent Application Publication
(10) Pub. No.: US 2023/0348866 A1
(43) Pub. Date: Nov. 2, 2023

(54) BIOSYNTHETIC PLATFORM FOR THE PRODUCTION OF CANNABINOIDS AND OTHER PRENYLATED COMPOUNDS

(71) Applicant: The Regents of the University of California, Oakland, CA (US)

(72) Inventors: James U. Bowie, Los Angeles, CA (US); Tyler P. Korman, Sierra Madre, CA (US); Meaghan Valliere, Leominster, MA (US)

(21) Appl. No.: 17/786,913

(22) PCT Filed: Dec. 24, 2020
(86) PCT No.: PCT/US2020/067032

§ 371 Date: Jun. 17, 2022

Related U.S. Application Data
(60) Provisional application No. 62/953,719, filed on Dec. 26, 2019.

(51) Int. Cl. C12N 9/10; C12P 7/6427; C12N 15/52

(52) U.S. Cl. CPC C12N 9/1085; C12Y 205/01039; C12P 7/6427; C12N 15/52

(57) ABSTRACT

Provided is an enzyme useful for prenylation and recombinant pathways for the production of cannabinoids, cannabinoid precursors and other prenylated chemicals in a cell free system as well and recombinant microorganisms that catalyze the reactions.

### CROSS REFERENCE TO RELATED APPLICATIONS

This application claims priority to U.S. Provisional Application Ser. No. 62/953,719, filed Dec. 26, 2019, the disclosures of which are incorporated herein by reference in their entirety.

### STATEMENT REGARDING FEDERALLY SPONSORED RESEARCH

This invention was made with government support under Grant Number DE-AR0000556, awarded by the U.S. Department of Energy. The government has certain rights in the invention.

### SEQUENCE LISTING

The instant application contains a Sequence Listing which has been submitted electronically in ASCII format and is hereby incorporated by reference in its entirety. Said ASCII copy, created on Dec. 24, 2020, is named Sequence-Listing_ST25.txt and is 207,506 bytes in size.

### TECHNICAL FIELD

Provided are methods of producing cannabinoids and other prenylated chemicals and compounds by contacting a suitable substrate with a metabolically-modified microorganism or enzymatic preparations or composition of the disclosure.

### BACKGROUND

Prenylation of natural compounds adds structural diversity, alters biological activity, and enhances therapeutic potential. Prenylated compounds often have low natural abundance or are difficult to isolate. Some prenylated natural products include a large class of bioactive molecules with demonstrated medicinal properties. Examples include prenyl-flavanoids, prenyl-stilbenoids, and cannabinoids

Cannabinoids are a large class of bioactive plant derived natural products that regulate the cannabinoid receptors (CB1 and CB2) of the human endocannabinoid system. Cannabinoids are promising pharmacological agents with over 100 ongoing clinical trials investigating their therapeutic benefits as antiemetics, anticonvulsants, analgesics and antidepressants. Further, three cannabinoid therapies have been FDA approved to treat chemotherapy induced nausea, MS spasticity and seizures associated with severe epilepsy.

Despite their therapeutic potential, the production of pharmaceutical grade (>99%) cannabinoids still face major technical challenges. Cannabis plants like marijuana and hemp produce high levels of tetrahydrocannabinolic (THCA) and cannabidiolic acid (CBDA), along with a variety of lower abundance cannabinoids. However, even highly expressed cannabinoids like CBDA and THCA, are challenging to isolate due to the high structural similarity of contaminating cannabinoids and the variability of cannabinoid composition with each crop. These problems are magnified when attempting to isolate rare cannabinoids. Moreover, current cannabis farming practices present serious environmental challenges. Consequently, there is considerable interest in developing alternative methods for the production of cannabinoids and cannabinoid analogs.

### SUMMARY

The disclosure provides an artificial in vitro enzymatic pathway for the production of CBG(V)A, the pathway comprising: (a) (1) an enzyme that converts prenol and ATP to prenol phosphate and ADP, an enzyme that converts prenol phosphate and ATP to dimethylallyl diphosphate (DMAPP), and/or (2) an enzyme that converts isoprenol and ATP to isoprenol phosphate and ADP and an enzyme that converts isoprenol phosphate and ATP to isopentenyl diphosphate (IPP); (b) an enzyme that isomerizes DMAPP to IPP and/or IPP to DMAPP; (c) an enzyme that converts DMAPP and IPP to geranyl pyrophosphate (GPP); and (d) an enzyme that converts GPP and olivetolic acid or divarinic acid or similar compound to CBG(V)A or variant thereof. In one embodiment, the input substrate(s) are olivetolic acid or divarinic acid, prenol and/or isoprenol. In another or further embodiment, the pathway comprises an ATP generating system that converts that ADP from part (a) to ATP.

The disclosure also provides an enzymatic scheme or pathway as set forth in FIG. 1A-B.

The disclosure also provides a recombinant polypeptide comprising a sequence selected from the group consisting of: (i) SEQ ID NO:30 and having a Y288X, A232S and a mutation selected from the group consisting of T69P, T98I and G224S, any combination of the foregoing and all of the foregoing mutations, wherein X is A, N, S, V or a non-natural amino acid; (ii) SEQ ID NO:30 and having a Y288X, A232S and a mutation selected from the group consisting of T69P, T98I, G224S and T126P, any combination of the foregoing and all of the foregoing mutations, wherein X is A, N, S, V or a non-natural amino acid; (iii) SEQ ID NO:30 and having a Y288X, A232S and a mutation selected from the group consisting of M14I, Y31W, T69P, T77I, T98I, S136A, E222D, G224S, N236T, G297K, any combination of the foregoing and all of the foregoing mutations, wherein X is A, N, S, V or a non-natural amino acid; (iv) SEQ ID NO:30 having a Y288X, A232S and a mutation selected from the group consisting of M14I, Y31W, T69P, T77I, E80A, D93S, T98I, T126P, M129L, G131Q, S136A, E222D, G224S, N236T, S277T, G297K, any combination of the foregoing and all of the foregoing mutations, wherein X is A, N, S, V or a non-natural amino acid; (v) SEQ ID NO:30 having a Y288X, A232S and a mutation selected from the group consisting of M14I, L33I, Y31W, T69P, T77I, V78A, E80A, D93S, T98I, E112G, T114V, T126P, M129L, G131Q, S136A, E222D, G224S, K225Q, N236T, S277T, G297K, any combination of the foregoing and all of the foregoing mutations, wherein X is A, N, S, V or a non-natural amino acid; (vi) any of (i)-(iv) or (v) comprising from 1-20 conservative amino acid substitutions and having NphB activity; (vii) a sequence that is at least 85%, 90%, 95%, 98% or 99% identical to the sequences of (i)-(iv) or (v) and which have NphB activity.

The disclosure also provides a method of producing CBG(V)A from GPP and Olivetolate (OA) or divirinic acid (DA) or CBGXA from GPP and a 2,4-dihydroxy benzoic acid or derivative thereof comprising incubating GPP and OA or DA, or GPP and 2,4-dihydroxy benzoic acid derivative with a recombinant polypeptide of the disclosure under condition to produce CBG(V)A or CBG(X)A, respectively.

The disclosure also provides a recombinant pathway comprising a polypeptide of the disclosure and a plurality of enzymes that convert prenol or isoprenol to geranylpyrophosphate (GPP). In one embodiment, the pathway further comprises an ATP regeneration module. In another or further embodiment, the ATP regeneration module converts acetyl-phosphate to acetic acid. In yet another or further embodiment of any of the foregoing embodiments, the pathway comprises the following enzymes (i) Acetyl-phosphate transferase (PTA); (ii) malonate decarboxylase alpha subunit (mdcA); (iii) acyl activating enzyme 3 (AAE3); (iv) olivetol synthase (OLS); (v) olivetolic acid cyclase (OAC); (vi) hydroxyethylthiazole kinase (ThiM); (vii) isopentenyl kinase (IPK); (viii) isopentyl diphosphate isomerase (IDI); (ix) Diphosphomevalonate decarboxylase alpha subunit (MDCa); (x) Geranyl-PP synthase (GPPS) or Farnesyl-PP synthease mutant S82F (FPPS S82F); and (xi) a recombinant polypeptide of the disclosure having prenylating activity. In another or further embodiment, the pathway is supplemented with BSA. In yet another embodiment, the pathway is supplemented with acetyl-phosphate, malonate, hexanoate or butyrate and isoprenol or prenol. In still another or further embodiment, the pathway further comprises a cannabidiolic acid synthase. In another or further embodiment, the pathway produces cannabidiolic acid.

The disclosure also provides a recombinant pathway comprising a recombinant polypeptide of the disclosure having prenylating activity and a plurality of enzymes that convert prenol or isoprenol to geranyl pyrophosphate (GPP).

The disclosure also provides a cell free enzymatic system for the production of geranyl pyrophosphate, the pathway including (i) Acetyl-phosphate transferase (PTA); (ii) malonate decarboxylase alpha subunit (mdcA); (iii) acyl activating enzyme 3 (AAE3); (iv) olivetol synthase (OLS); (v) olivetolic acid cyclase (OAC); (vi) hydroxyethylthiazole kinase (ThiM); (vii) isopentenyl kinase (IPK); (viii) isopentyl diphosphate isomerase (IDI); (ix) Diphosphomevalonate decarboxylase alpha subunit (MDCa); (x) Geranyl-PP synthase (GPPS) or Farnesyl-PP synthease mutant S82F (FPPS S82F); and (xi) a recombinant polypeptide comprising a sequence selected from the group consisting of: (a) SEQ ID NO:30 and having a Y288X, A232S and a mutation selected from the group consisting of T69P, T98I and G224S, any combination of the foregoing and all of the foregoing mutations, wherein X is A, N, S, V or a non-natural amino acid; (b) SEQ ID NO:30 and having a Y288X, A232S and a mutation selected from the group consisting of T69P, T98I, G224S and T126P, any combination of the foregoing and all of the foregoing mutations, wherein X is A, N, S, V or a non-natural amino acid; (c) SEQ ID NO:30 and having a Y288X, A232S and a mutation selected from the group consisting of M14I, Y31W, T69P, T77I, T98I, S136A, E222D, G224S, N236T, G297K, any combination of the foregoing and all of the foregoing mutations, wherein X is A, N, S, V or a non-natural amino acid; (d) SEQ ID NO:30 having a Y288X, A232S and a mutation selected from the group consisting of M14I, Y31W, T69P, T77I, E80A, D93S, T98I, T126P, M129L, G131Q, S136A, E222D, G224S, N236T, S277T, G297K, any combination of the foregoing and all of the foregoing mutations, wherein X is A, N, S, V or a non-natural amino acid; (e) SEQ ID NO:30 having a Y288X, A232S and a mutation selected from the group consisting of M14I, L33I, Y31W, T69P, T77I, V78A, E80A, D93S, T98I, E112G, T114V, T126P, M129L, G131Q, S136A, E222D, G224S, K225Q, N236T, S277T, G297K, any combination of the foregoing and all of the foregoing mutations, wherein X is A, N, S, V or a non-natural amino acid; (f) any of (a)-(d) or (e) comprising from 1-20 conservative amino acid substitutions and having NphB activity; (g) a sequence that is at least 85%, 90%, 95%, 98% or 99% identical to the sequences of (a)-(d) or (e) and which have NphB activity.

The disclosure also provides an isolated polynucleotide encoding a polypeptide selected from the group consisting of: (i) SEQ ID NO:30 and having a Y288X, A232S and a mutation selected from the group consisting of T69P, T98I and G224S, any combination of the foregoing and all of the foregoing mutations, wherein X is A, N, S, V or a non-natural amino acid; (ii) SEQ ID NO:30 and having a Y288X, A232S and a mutation selected from the group consisting of T69P, T98I, G224S and T126P, any combination of the foregoing and all of the foregoing mutations, wherein X is A, N, S, V or a non-natural amino acid; (iii) SEQ ID NO:30 and having a Y288X, A232S and a mutation selected from the group consisting of M14I, Y31W, T69P, T77I, T98I, S136A, E222D, G224S, N236T, G297K, any combination of the foregoing and all of the foregoing mutations, wherein X is A, N, S, V or a non-natural amino acid; (iv) SEQ ID NO:30 having a Y288X, A232S and a mutation selected from the group consisting of M14I, Y31W, T69P, T77I, E80A, D93S, T98I, T126P, M129L, G131Q, S136A, E222D, G224S, N236T, S277T, G297K, any combination of the foregoing and all of the foregoing mutations, wherein X is A, N, S, V or a non-natural amino acid; (v) SEQ ID NO:30 having a Y288X, A232S and a mutation selected from the group consisting of M14I, L33I, Y31W, T69P, T77I, V78A, E80A, D93S, T98I, E112G, T114V, T126P, M129L, G131Q, S136A, E222D, G224S, K225Q, N236T, S277T, G297K, any combination of the foregoing and all of the foregoing mutations, wherein X is A, N, S, V or a non-natural amino acid; (vi) any of (i)-(iv) or (v) comprising from 1-20 conservative amino acid substitutions and having NphB activity; (vii) a sequence that is at least 85%, 90%, 95%, 98% or 99% identical to the sequences of (i)-(iv) or (v) and which have NphB activity.

The disclosure also provides a vector comprising an isolated polynucleotide of the disclosure.

The disclosure also provides a recombinant microorganism comprising the isolated polynucleotide of the disclosure or vector of the disclosure.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the disclosure and, together with the detailed description, serve to explain the principles and implementations of the invention.

FIG. 1A-B show a cell-free system design for cannabinoid production of the disclosure. (A) GPP is derived from isoprenoid module pathway (dark blue path; top left). The aromatic polyketide OA or DA is derived from hexanoate (or butyrate) and malonate (green path). Malonyl-CoA is generated from malonate via a non-natural transfer of CoA from acetyl-CoA using MdcA (starred). Acetyl-CoA is derived from acetyl phosphate, which is also used to regenerate ATP (red path; top right). The aromatic polyketide is prenylated from GPP derived from the isoprenoid module using a designed CBGA synthase, which yields the CBG(V)A cannabinoids. Although not part of the cell free system, the figure illustrates how CBG(V)A can be converted into many additional medicinally interesting cannabinoids in a single enzymatic step. Enzymes and abbreviations used are listed in Table 1. (B) shows an alternative depiction of a pathway of the disclosure. R=alkyl group; inputs are aromatic polyketides such as olivetolate, prenol or isoprenol, or both prenol and isoprenol. When both prenol and isoprenol are used, IDI is not necessary; different ATP generating systems could be used, including but not limited to methods described in Zhao et al., “Regeneration of cofactors for use in biocatalysis,” Curr Opin Biotechnol., 14(6):583-9, 2003.

FIG. 2A-F shows testing OA/DA synthesis. (A) The simplified MatB pathway for testing OA/DA production. (B) The OA (squares) or DA (circles) titer over time using the MatB path. (C) The effect of additives on OA or DA production using the MatB pathway. Additives were added to a reaction at time zero, and the titer of OA or DA at 4 hours relative to the control is plotted. Error bars represent standard deviation of biological replicates. (D) Scheme for OA/DA production from hexanoate, malonate and AcP using MdcA to generate malonyl-CoA. (E) Production of the aromatic polyketides OA (squares) and DA (circles) using the MdcA system in panel D. The time course was carried out in the presence (filled shape) or absence (outlined shape) of BSA. (F) CBGA (squares) and CBGVA (circles) production from isoprenol and added OA or DA, respectively. Error bars represent standard deviation of biological replicates.

FIG. 3A-C shows implementation of the full cannabinoid production system. (A) Time course for conversion of inputs isoprenol, acetyl phosphate, malonate and hexanoate (or butyrate) into CBGA (squares) or CBGVA (circles). (B) Production of intermediates in the full system. A reaction producing CBGA was monitored for OA production (black circles), CBGA production (green triangles) and GPP production (blue squares). (C) Enzyme recycling. At 6 hours the enzymes from a CBGA producing reaction were concentrated and washed to remove metabolites. A new reaction was set up with fresh inputs and co-factors, and the reaction was quenched after an addition 31 hours. The titer of the initial reaction (Initial) and total titer of the initial and recycled reaction is shown (Recycled Enzymes). Error bars represent standard deviation of biological replicates.

FIG. 4 shows the effect of OLS and AAE3 concentrations on product specificity. The concentration of CsOLS vs Product Specificity is plotted at three different AAE3 concentrations. As the concentration of CsOLS or CsAAE3 increased, a decrease in product specificity was observed.

FIG. 5A-B shows OA and DA inhibition of enzyme activity. (A) The percent activity remaining at 5 mM OA (blue) and DA (green) compared to no addition is shown for 4 enzymes. (B) At reaction relevant conditions, CsOLS is the most inhibited by OA.

FIG. 6 shows inhibition of OA and CBGA production by GPP. The RpMatB reaction system was used to generate OA, which can then be prenylated by the added GPP, catalyzed by NphBM31^{S}. Increasing GPP leads to a decrease in overall production of OA and CBGA, indicating that GPP inhibits the OA pathway.

FIG. 7 shows the titer of CBGA as a function of initial AcP concentrations. A 50 mM initial AcP concentration was used because increasing the AcP concentration over 50 mM decreases the CBGA titer.

FIG. 8 shows the effect of BSA on the titer of OA using MdcA to generate malonyl-CoA. BSA titration data showing 20 mg/mL BSA should be used in subsequent reactions because there was minimal improvement when BSA was increased to 40 mg/mL.

FIG. 9 shows the effect of acetate and phosphate on CBGA production. Varying starting Acetate or Phosphate concentration from 0 to 100 mM had minimal effect on CBGA production using isoprenol and OA as inputs.

FIG. 10 shows the stabilization of NphB M31. Activity remaining after a 20 min incubation at various temperatures is shown for the parent enzyme NphB M31 and the new enzyme NphB M31^{s}.

### DETAILED DESCRIPTION

As used herein and in the appended claims, the singular forms “a,” “an,” and “the” include plural referents unless the context clearly dictates otherwise. Thus, for example, reference to “a polynucleotide” includes a plurality of such polynucleotides and reference to “the enzyme” includes reference to one or more enzymes, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Any publications discussed above and throughout the text are provided solely for their disclosure prior to the filing date of the present application. Nothing herein is to be construed as an admission that the inventors are not entitled to antedate such disclosure by virtue of prior disclosure.

As used herein, an “activity” of an enzyme is a measure of its ability to catalyze a reaction resulting in a metabolite, i.e., to “function”, and may be expressed as the rate at which the metabolite of the reaction is produced. For example, enzyme activity can be represented as the amount of metabolite produced per unit of time or per unit of enzyme (e.g., concentration or weight), or in terms of affinity or dissociation constants.

“Bacteria”, or “eubacteria”, refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; and (11) Thermotoga and Thermosipho thermophiles.

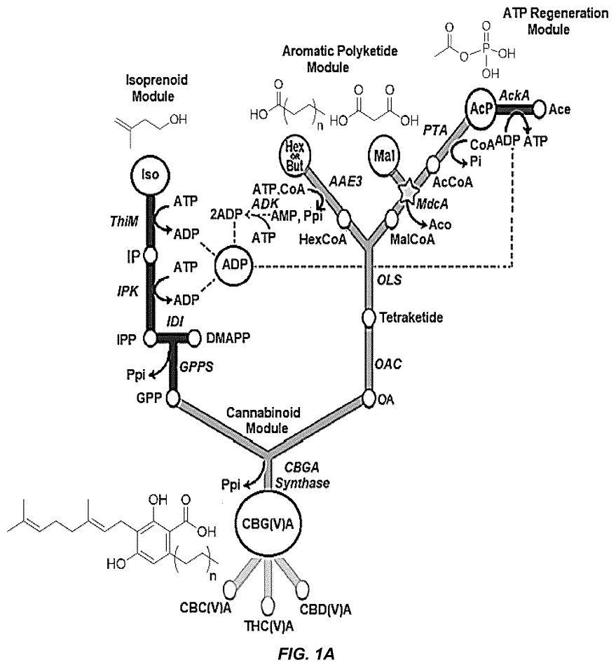

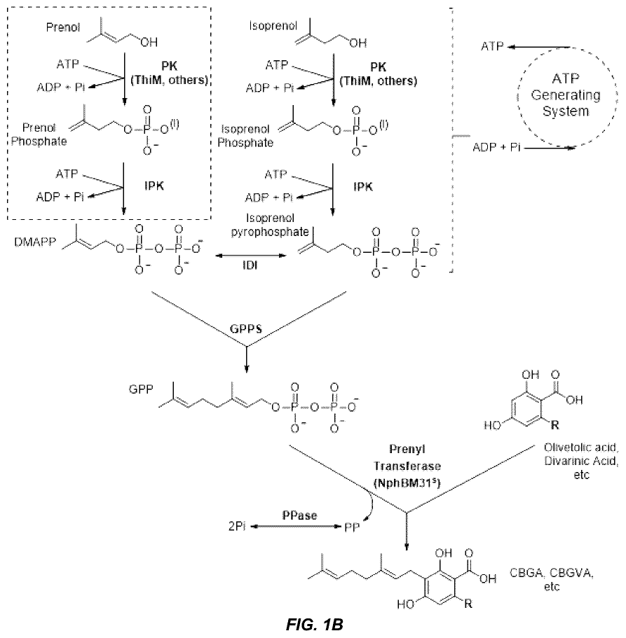

The term “biosynthetic pathway”, also referred to as “metabolic pathway”, refers to a set of anabolic or catabolic biochemical reactions for converting (transmuting) one chemical species into another (see, e.g., FIG. 1). Gene products belong to the same “metabolic pathway” if they, in parallel or in series, act on the same substrate, produce the same product, or act on or produce a metabolic intermediate (i.e., metabolite) between the same substrate and metabolite end product. The disclosure provides recombinant microorganism having a metabolically engineered pathway for the production of a desired product or intermediate.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

An “enzyme” means any substance, typically composed wholly or largely of amino acids making up a protein or polypeptide that catalyzes or promotes, more or less specifically, one or more chemical or biochemical reactions.

The term “expression” with respect to a gene or polynucleotide refers to transcription of the gene or polynucleotide and, as appropriate, translation of the resulting mRNA transcript to a protein or polypeptide. Thus, as will be clear from the context, expression of a protein or polypeptide results from transcription and translation of the open reading frame.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram positive bacteria” include cocci, nonsporulating rods, and sporulating rods. The genera of gram positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences).

As used herein, two proteins (or a region of the proteins) are substantially homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (see, e.g., Pearson et al., 1994, hereby incorporated herein by reference).

In addition, and as mentioned above, homologs of enzymes useful for generating metabolites are encompassed by the microorganisms and methods provided herein. The term “homologs” used with respect to an original enzyme or gene of a first family or species refers to distinct enzymes or genes of a second family or species which are determined by functional, structural or genomic analyses to be an enzyme or gene of the second family or species which corresponds to the original enzyme or gene of the first family or species. Most often, homologs will have functional, structural or genomic similarities. Techniques are known by which homologs of an enzyme or gene can readily be cloned using genetic probes and PCR. Identity of cloned sequences as homolog can be confirmed using functional assays and/or by genomic mapping of the genes.

Sequence homology for polypeptides, which can also be referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1.

A typical algorithm used comparing a molecule sequence to a database containing a large number of sequences from different organisms is the computer program BLAST (Altschul, 1990; Gish, 1993; Madden, 1996; Altschul, 1997; Zhang, 1997), especially blastp or tblastn (Altschul, 1997). Typical parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

When searching a database containing sequences from a large number of different organisms, it is typical to compare amino acid sequences. Database searching using amino acid sequences can be measured by algorithms other than BLASTp known in the art. For instance, polypeptide sequences can be compared using FASTA, a program in GCG Version 6.1. FASTA provides alignments and percent sequence identity of the regions of the best overlap between the query and search sequences (Pearson, 1990, hereby incorporated herein by reference). For example, percent sequence identity between amino acid sequences can be determined using FASTA with its default parameters (a word size of 2 and the PAM250 scoring matrix), as provided in GCG Version 6.1, hereby incorporated herein by reference.

In some instances “isozymes” can be used that carry out the same functional conversion/reaction, but which are so dissimilar in structure that they are typically determined to not be “homologous”.

As used herein, the term “metabolically engineered” or “metabolic engineering” involves rational pathway design and assembly of biosynthetic genes, genes associated with operons, and control elements of such polynucleotides, for the production of a desired metabolite, such as an GPP and/or OA, CBG(V)A or other chemical, in a microorganism, partially in a microorganism, in a cell free system and/or a combination of cell-free system and microorganism. “Metabolically engineered” can further include optimization of metabolic flux by regulation and optimization of transcription, translation, protein stability and protein functionality using genetic engineering and appropriate culture condition including the reduction of, disruption, or knocking out of, a competing metabolic pathway that competes with an intermediate leading to a desired pathway. A biosynthetic gene can be heterologous to the host microorganism, either by virtue of being foreign to the host, or being modified by mutagenesis, recombination, and/or association with a heterologous expression control sequence in an endogenous host cell. In one embodiment, where the polynucleotide is xenogenetic to the host organism, the polynucleotide can be codon optimized.

A “metabolite” refers to any substance produced by metabolism or enzymatic pathway or a substance necessary for or taking part in a particular metabolic process or pathway that gives rise to a desired metabolite, chemical, etc. A metabolite can be an organic compound that is a starting material (e.g., isoprenol etc.), an intermediate in (e.g., IP), or an end product (e.g., GPP) of metabolism or enzymatic pathway. Metabolites can be used to construct more complex molecules, or they can be broken down into simpler ones. Intermediate metabolites may be synthesized from other metabolites, perhaps used to make more complex substances, or broken down into simpler compounds, often with the release of chemical energy.

The term “microorganism” includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

A “mutation” means any process or mechanism resulting in a mutant protein, enzyme, polynucleotide, gene, or cell. This includes any mutation in which a protein, enzyme, polynucleotide, or gene sequence is altered, and any detectable change in a cell arising from such a mutation. Typically, a mutation occurs in a polynucleotide or gene sequence, by point mutations, deletions, or insertions of single or multiple nucleotide residues. A mutation includes polynucleotide alterations arising within a protein-encoding region of a gene as well as alterations in regions outside of a protein-encoding sequence, such as, but not limited to, regulatory or promoter sequences. A mutation in a gene can be “silent”, i.e., not reflected in an amino acid alteration upon expression, leading to a “sequence-conservative” variant of the gene. This generally arises when one amino acid corresponds to more than one codon. A mutation that gives rise to a different primary sequence of a protein can be referred to as a mutant protein or protein variant.

A “native” or “wild-type” protein, enzyme, polynucleotide, gene, or cell, means a protein, enzyme, polynucleotide, gene, or cell that occurs in nature.

A “parental microorganism” refers to a cell used to generate a recombinant microorganism. The term “parental microorganism” describes, in one embodiment, a cell that occurs in nature, i.e. a “wild-type” cell that has not been genetically modified. The term “parental microorganism” further describes a cell that serves as the “parent” for further engineering. In this latter embodiment, the cell may have been genetically engineered, but serves as a source for further genetic engineering.

For example, a wild-type microorganism can be genetically modified to express or over express a first target enzyme. This microorganism can act as a parental microorganism in the generation of a microorganism modified to express or over-express a second target enzyme. In turn, that microorganism can be modified to express or over express a third target enzyme, etc. As used herein, “express” or “over express” refers to the phenotypic expression of a desired gene product. In one embodiment, a naturally occurring gene in the organism can be engineered such that it is linked to a heterologous promoter or regulatory domain, wherein the regulatory domain causes expression of the gene, thereby modifying its normal expression relative to the wild-type organism. Alternatively, the organism can be engineered to remove or reduce a repressor function on the gene, thereby modifying its expression. In yet another embodiment, a cassette comprising the gene sequence operably linked to a desired expression control/regulatory element is engineered in to the microorganism.

Accordingly, a parental microorganism functions as a reference cell for successive genetic modification events. Each modification event can be accomplished by introducing one or more nucleic acid molecules into the reference cell. The introduction facilitates the expression or over-expression of one or more target enzyme or the reduction or elimination of one or more target enzymes. It is understood that the term “facilitates” encompasses the activation of endogenous polynucleotides encoding a target enzyme through genetic modification of e.g., a promoter sequence in a parental microorganism. It is further understood that the term “facilitates” encompasses the introduction of exogenous polynucleotides encoding a target enzyme into a parental microorganism.

A “parental enzyme or protein” refers to an enzyme or protein used to generate a variant or mutant enzyme or protein. The term “parental enzyme” (or protein) describes, in one embodiment, an enzyme or protein that occurs in nature, i.e. a “wild-type” enzyme or protein that has not been genetically modified. The term “parental enzyme” (or protein) further describes a cell that serves as the “parent” for further engineering. In this latter embodiment, the enzyme or protein may have been genetically engineered, but serves as a source for further genetic engineering.

The term “polynucleotide,” “nucleic acid” or “recombinant nucleic acid” refers to polynucleotides such as deoxyribonucleic acid (DNA), and, where appropriate, ribonucleic acid (RNA).

Polynucleotides that encode enzymes useful for generating metabolites including homologs, variants, fragments, related fusion proteins, or functional equivalents thereof, are used in recombinant nucleic acid molecules that direct the expression of such polypeptides in appropriate host cells, such as bacterial or yeast cells. The sequences provided herein and the accession numbers provide those of skill in the art the ability to obtain and obtain coding sequences for various enzymes of the disclosure using readily available software and basic biology knowledge.

Those of skill in the art will recognize that, due to the degenerate nature of the genetic code, a variety of codons differing in their nucleotide sequences can be used to encode a given amino acid. A particular polynucleotide or gene sequence encoding a biosynthetic enzyme or polypeptide described above are referenced herein merely to illustrate an embodiment of the disclosure, and the disclosure includes polynucleotides of any sequence that encode a polypeptide comprising the same amino acid sequence of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with alternate amino acid sequences, and the amino acid sequences encoded by the DNA sequences shown herein merely illustrate exemplary embodiments of the disclosure.

The disclosure provides polynucleotides in the form of recombinant DNA expression vectors or plasmids, as described in more detail elsewhere herein, that encode one or more target enzymes. Generally, such vectors can either replicate in the cytoplasm of the host microorganism or integrate into the chromosomal DNA of the host microorganism. In either case, the vector can be a stable vector (i.e., the vector remains present over many cell divisions, even if only with selective pressure) or a transient vector (i.e., the vector is gradually lost by host microorganisms with increasing numbers of cell divisions). The disclosure provides DNA molecules in isolated (i.e., not pure, but existing in a preparation in an abundance and/or concentration not found in nature) and purified (i.e., substantially free of contaminating materials or substantially free of materials with which the corresponding DNA would be found in nature) form.

A polynucleotide of the disclosure can be amplified using cDNA, mRNA or alternatively, genomic DNA, as a template and appropriate oligonucleotide primers according to standard PCR amplification techniques and those procedures described in the Examples section below. The nucleic acid so amplified can be cloned into an appropriate vector and characterized by DNA sequence analysis. Furthermore, oligonucleotides corresponding to nucleotide sequences can be prepared by standard synthetic techniques, e.g., using an automated DNA synthesizer.

The disclosure provides a number of polypeptide sequences in the sequence listing accompanying the present application, which can be used to design, synthesize and/or isolate polynucleotide sequences using the degeneracy of the genetic code or using publicly available databases to search for the coding sequences.

It is also understood that an isolated polynucleotide molecule encoding a polypeptide homologous to the enzymes described herein can be created by introducing one or more nucleotide substitutions, additions or deletions into the nucleotide sequence encoding the particular polypeptide, such that one or more amino acid substitutions, additions or deletions are introduced into the encoded protein. Mutations can be introduced into the polynucleotide by standard techniques, such as site-directed mutagenesis and PCR-mediated mutagenesis. In contrast to those positions where it may be desirable to make a non-conservative amino acid substitution, in some positions it is preferable to make conservative amino acid substitutions.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, a process sometimes called “codon optimization” or “controlling for species codon bias.”

Optimized coding sequences containing codons preferred by a particular prokaryotic or eukaryotic host (see also, Murray et al. (1989) Nucl. Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al. (1996) Nucl. Acids Res. 24: 216-218). Methodology for optimizing a nucleotide sequence for expression in a plant is provided, for example, in U.S. Pat. No. 6,015,891, and the references cited therein.

It is understood that a polynucleotide described herein include “genes” and that the nucleic acid molecules described above include “vectors” or “plasmids.”

The term “prokaryotes” is art recognized and refers to cells which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

A “protein” or “polypeptide”, which terms are used interchangeably herein, comprises one or more chains of chemical building blocks called amino acids that are linked together by chemical bonds called peptide bonds. A protein or polypeptide can function as an enzyme.

The term “substrate” or “suitable substrate” refers to any substance or compound that is converted or meant to be converted into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a starting material, but also intermediate and end product metabolites used in a pathway associated with a metabolically engineered microorganism as described herein.

“Transformation” refers to the process by which a vector is introduced into a host cell. Transformation (or transduction, or transfection), can be achieved by any one of a number of means including electroporation, microinjection, biolistics (or particle bombardment-mediated delivery), or Agrobacterium mediated transformation.

A “vector” generally refers to a polynucleotide that can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes,” that is, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an Agrobacterium or a bacterium.

The various components of an expression vector can vary widely, depending on the intended use of the vector and the host cell(s) in which the vector is intended to replicate or drive expression. Expression vector components suitable for the expression of genes and maintenance of vectors in E. coli, yeast, Streptomyces, and other commonly used cells are widely known and commercially available. For example, suitable promoters for inclusion in the expression vectors of the disclosure include those that function in eukaryotic or prokaryotic host microorganisms. Promoters can comprise regulatory sequences that allow for regulation of expression relative to the growth of the host microorganism or that cause the expression of a gene to be turned on or off in response to a chemical or physical stimulus. For E. coli and certain other bacterial host cells, promoters derived from genes for biosynthetic enzymes, antibiotic-resistance conferring enzymes, and phage proteins can be used and include, for example, the galactose, lactose (lac), maltose, tryptophan (trp), beta-lactamase (bla), bacteriophage lambda PL, and T5 promoters. In addition, synthetic promoters, such as the tac promoter (U.S. Pat. No. 4,551,433, which is incorporated herein by reference in its entirety), can also be used. For E. coli expression vectors, it is useful to include an E. coli origin of replication, such as from pUC, p1P, p1, and pBR.

Thus, recombinant expression vectors contain at least one expression system, which, in turn, is composed of at least a portion of a gene coding sequences operably linked to a promoter and optionally termination sequences that operate to effect expression of the coding sequence in compatible host cells. The host cells are modified by transformation with the recombinant DNA expression vectors of the disclosure to contain the expression system sequences either as extrachromosomal elements or integrated into the chromosome.

The disclosure provides accession numbers and sequences for various genes, homologs and variants useful in the generation of recombinant microorganism and proteins for use in in vitro systems. It is to be understood that homologs and variants described herein are exemplary and non-limiting. Additional homologs, variants and sequences are available to those of skill in the art using various databases including, for example, the National Center for Biotechnology Information (NCBI) access to which is available on the World-Wide-Web.

It is well within the level of skill in the art to utilize the sequences and accession number described herein to identify homologs and isozymes that can be used or substituted for any of the polypeptides used herein. In fact, a BLAST search of any one of the sequences provide herein will identify a plurality of related homologs.

The sequence listing accompanying this application provides exemplary polypeptides useful in the methods described herein. It is understood that the addition of sequences which do not alter the activity of a polypeptide molecule, such as the addition of a non-functional or non-coding sequence (e.g., polyHIS tags), is a conservative variation of the basic molecule.

Cannabinoids show immense therapeutic potential with over 100 ongoing clinical trials as antiemetics, anticonvulsants, antidepressants, anticancer and analgesics. Nevertheless, despite the therapeutic potential of prenyl-natural products, their study and use is limited by the lack of cost-effective production methods.

The two main alternatives to plant-based cannabinoid production are organic synthesis and production in a metabolically engineered host (e.g., plant, yeast, or bacteria). Total syntheses have been elucidated for the production of some cannabinoids, such as THCA and CBDA, but they are often not practical for drug manufacturing. Additionally, the synthetic approach is not modular, requiring a unique synthesis for each cannabinoid. A modular approach could be achieved by using the natural biosynthetic pathway.

The three major cannabinoids (THCA, CBDA and cannabichromene or CBCA) are derived from a single precursor, CBGA. Additionally, three low abundance cannabinoids are derived from CBGVA (FIG. 1). Thus, the ability to make CBGA and CBGVA in a heterologous host would open the door to the production of an array of cannabinoids. Unfortunately, engineering microorganisms to produce CBGA and CBGVA has proven extremely challenging.

Cannabinoids are derived from a combination of fatty acid, polyketide, and terpene biosynthetic pathways that generate the key building blocks geranyl pyrophosphate (GPP) and olivetolic acid (OA) (FIG. 1). High level CBGA biosynthesis requires the re-routing of long, essential and highly regulated pathways. Moreover, GPP is toxic to cells, creating a notable barrier to high level production in microbes.

Synthetic biochemistry, in which complex biochemical conversions are performed cell-free using a mixture of enzymes, affords potential advantages over traditional metabolic engineering including: a higher level of flexibility in pathway design; greater control over component optimization; more rapid design-build-test cycles; and freedom from cell toxicity of intermediates or products. The disclosure provides a cell-free system for the production of cannabinoids. It should be noted the “full” pathway does not need to be in a cell free system (i.e., parts of the pathway can be performed in cells, and their products provided to a cell-free system) or vice-a-versa.

This disclosure provides enzyme variants and pathways comprising such variants for the production of cannabinoids. In addition, the biosynthetic pathways described herein use “purge valves” or “regeneration valves” to regulate co-factor availability (e.g., ATP, NADH/NAD⁺, and NADPH/NADP⁺ levels).

The disclosure provides a cell-free system for the production of the central cannabinoids CBGVA and CBGA (abbr. CBG(V)A herein), because many other key cannabinoids can be obtained from CBG(V)A in single, well-established enzymatic steps (FIG. 1). The metabolic pathway of the disclosure can be broken down into various modules. The Isoprenoid (ISO) module builds geranyl pyrophosphate (GPP) from isoprenol using a simplified isoprenoid pathway. The Aromatic Polyketide (AP) module converts the inputs malonate and hexanoate (or butyrate) into olivetolic acid (OA) or divarinic acid (DA). Other fatty acid inputs could be utilized as well to make related aromatic polyketides. The Cannabinoid (CAN) module receives the GPP from the ISO module and prenylates OA/DA from the AP module to produce the central cannabinoids CBG(V)A. The entire system is powered by ATP that is made in the ATP Regeneration (AR) module. Acetyl phosphate (AcP) was used as a sacrificial substrate for ATP regeneration because it can be made inexpensively from acetic anhydride and phosphoric acid. Other methods for generating ATP using sacrificial substrates could be used and are well known in the literature (see, e.g., Zhao H, et al., “Regeneration of cofactors for use in biocatalysis,” Curr Opin Biotechnol. 14(6):583-9, 2003).

To reduce ATP requirements, the pathway uses a non-natural route for malonyl-CoA production as a “regeneration valve”. Normally malonyl-CoA generation from malonate requires 2 ATP equivalents per malonate employed, via the action of the enzyme malonyl-CoA synthetase (MatB; SEQ ID NO:16 or sequences having at least 85% identity thereto, e.g., 85%, 87%, 90%, 92%, 95%, 98%, 99% or 100%). Since three malonate are required per OA/DA produced, the ATP contribution for malonate activation is 6 ATP. To lower the ATP requirement, the disclosure provides a way to directly transfer CoA from acetyl-CoA to malonate, making acetate and malonyl-CoA, since the thioester transfer should be thermodynamically favorable. Because acetyl-CoA can be directly derived from the input AcP with phosphotransacetylase this approach would save 3 ATP-equivalents per OA/DA. While there is no natural enzyme that performs the transferase reaction, the isolated a subunit of the enzyme malonate decarboxylase (MdcA) can fortuitously catalyze this reaction when expressed in isolation. Thus, the disclosure incorporates MdcA (or homolog thereof; SEQ ID NO:6 or sequences having at least 50% or more sequence identity thereto) into the overall pathway design.

A synthetic biochemistry approach is outlined in FIG. 1. In one embodiment, GPP is derived from isoprenol or prenol. In one embodiment, GPP is derived from isoprenol. In yet a further embodiment, the isoprenol pathway to GPP is coupled to an ATP regeneration system. For example, the pathway can be coupled with a creatine kinase ATP generating system; an acetate kinase system; a glycolysis system as well as others. In one embodiment, the ATP regeneration system comprises an acetate kinase. Enzymes (nucleic acid coding sequences and polypeptides) of FIG. 1 are provided in SEQ ID NOs: 54-65 (e.g., PRK enzymes are provided in SEQ ID NOs: 54-57; IPK enzymes are provided in SEQ ID NOs: 58-61; IDI enzymes are provided in SEQ ID NOs:20-27 and 62-63; and FPPS enzymes are provided in SEQ ID NOs: 64-65).

NphB is an aromatic prenyltransferase that catalyzes the attachment of a 10-carbon geranyl group to aromatic substrates. NphB exhibits a rich substrate selectivity and product regioselectivity. NphB, identified from Streptomyces, catalyzes the addition of a 10-carbon geranyl group to a number of small organic aromatic substrates. NphB has a spacious and solvent accessible binding pocket in to which two substrates molecules, geranyl diphosphate (GPP) and 1,6-dihydroxynaphthalene (1,6-DHN), can be bound. GPP is stabilized via interactions between its negatively charged diphosphate moiety and several amino acid sidechains, including Lys119, Thr/Gln171, Arg228, Tyr216 and Lys284, in addition to Mg²⁺. A Mg²⁺ cofactor is required for the activity of NphB. NphB from Streptomyces has a sequence as set forth in SEQ ID NO:30.

NovQ (accession no. AAF67510, incorporated herein by reference) is a member of the CloQ/NphB class of prenyltransferases. The novQ gene can be cloned from Streptomyces niveus, which produces an aminocoumarin antibiotic, novobiocin. Recombinant NovQ can be expressed in Escherichia coli and purified to homogeneity. The purified enzyme is a soluble monomeric 40-kDa protein that catalyzed the transfer of a dimethylallyl group to 4-hydroxyphenylpyruvate (4-HPP) independently of divalent cations to yield 3-dimethylallyl-4-HPP, an intermediate of novobiocin. In addition to the prenylation of 4-HPP, NovQ catalyzed carbon-carbon-based and carbon-oxygen-based prenylations of a diverse collection of phenylpropanoids, flavonoids and dihydroxynaphthalenes. Despite its catalytic promiscuity, the NovQ-catalyzed prenylation occurred in a regiospecific manner. NovQ is the first reported prenyltransferase capable of catalyzing the transfer of a dimethylallyl group to both phenylpropanoids, such as p-coumaric acid and caffeic acid, and the B-ring of flavonoids. NovQ can serve as a useful biocatalyst for the synthesis of prenylated phenylpropanoids and prenylated flavonoids.

Aspergillus terreus aromatic prenyltransferase (AtaPT; accession no. AMB20850, incorporated herein by reference), is responsible for the prenylation of various aromatic compounds. Recombinant AtaPT can be overexpressed in Escherichia coli and purified. Aspergillus terreus aromatic prenyltransferase (AtaPT) catalyzes predominantly C-monoprenylation of acylphloroglucinols in the presence of different prenyl diphosphates.

Mutational experiments were performed on NphB to improve substrate specificity and stability. The disclosure provides an NphB mutant comprising SEQ ID NO:30 having a Y288X, A232S and a mutation selected from the group consisting of T69P, T98I and G224S, any combination of the foregoing and all of the foregoing mutations, wherein X is A, N, S, V or a non-natural amino acid; SEQ ID NO:30 and having a Y288X, A232S and a mutation selected from the group consisting of T69P, T98I, G224S and T126P any combination of the foregoing and all of the foregoing mutations, wherein X is A, N, S, V or a non-natural amino acid; SEQ ID NO:30 having a Y288X, A232S and a mutation selected from the group consisting of M14I, Y31W, T69P, T77I, T98I, S136A, E222D, G224S, N236T, G297K, any combination of the foregoing and all of the foregoing mutations; wherein X is A, N, S, V or a non-natural amino acid. In another embodiment, the disclosure provides an NphB mutant comprising SEQ ID NO:30 having a Y288X, A232S and a mutation selected from the group consisting of M14I, Y31W, T69P, T77I, E80A, D93S, T98I, T126P, M129L, G131Q, S136A, E222D, G224S, N236T, S277T, G297K, any combination of the foregoing and all of the foregoing mutations; wherein X is A, N, S, V or a non-natural amino acid. In another embodiment, the disclosure provides an NphB mutant comprising SEQ ID NO:30 having a Y288X, A232S and a mutation selected from the group consisting of M14I, L33I, Y31W, T69P, T77I, V78A, E80A, D93S, T98I, E112G, T114V, T126P, M129L, G131Q, S136A, E222D, G224S, K225Q, N236T, S277T, G297K, any combination of the foregoing and all of the foregoing mutations; wherein X is A, N, S, V or a non-natural amino acid.

The disclosure thus provides mutant NphB variants comprising (i) SEQ ID NO:30 having a Y288X, A232S and a mutation selected from the group consisting of T69P, T98I and G224S, any combination of the foregoing and all of the foregoing mutations, wherein X is A, N, S, V or a non-natural amino acid; (ii) SEQ ID NO:30 and having a Y288X, A232S and a mutation selected from the group consisting of T69P, T98I, G224S and T126P any combination of the foregoing and all of the foregoing mutations, wherein X is A, N, S, V or a non-natural amino acid; (iii) SEQ ID NO:30 and having a Y288X, A232S and a mutation selected from the group consisting of M14I, Y31W, T69P, T77I, T98I, S136A, E222D, G224S, N236T, G297K, any combination of the foregoing and all of the foregoing mutations, wherein X is A, N, S, V or a non-natural amino acid; (iv) SEQ ID NO:30 having a Y288X, A232S and a mutation selected from the group consisting of M14I, Y31W, T69P, T77I, E80A, D93S, T98I, T126P, M129L, G131Q, S136A, E222D, G224S, N236T, S277T, G297K, any combination of the foregoing and all of the foregoing mutations, wherein X is A, N, S, V or a non-natural amino acid; (v) SEQ ID NO:30 having a Y288X, A232S and a mutation selected from the group consisting of M14I, L33I, Y31W, T69P, T77I, V78A, E80A, D93S, T98I, E112G, T114V, T126P, M129L, G131Q, S136A, E222D, G224S, K225Q, N236T, S277T, G297K, any combination of the foregoing and all of the foregoing mutations, wherein X is A, N, S, V or a non-natural amino acid; (vi) any of (i)-(v) comprising from 1-20 (e.g., 2, 5, 10, 15 or 20; or any value between 1 and 20) conservative amino acid substitutions and having NphB activity; (vii) a sequence that is at least 85%, 90%, 95%, 98% or 99% identical to the sequences of any one of (i) to (v) and which have NphB activity. By “NphB activity” means the ability of the enzyme to prenylated a substrate and more specifically to generate CBGA from OA.

The following provides an alignment of various mutants (all of which had biological effect; SEQ ID NOs:40, 41, 42, 43, 44) and wildtype sequence (SEQ ID NO:30):

| | | |
|---|---|---|
| 1ZB6_designed_4_a | MSEAADVERVYAAIEEAAGLLGVACARDKIWPLLSTFQDTLVEGGSVVVFSMASGRHSTE | 60 |
| 1ZB6_designed_5_a | MSEAADVERVYAAIEEAAGLLGVACARDKIWPLLSTFQDTLVEGGSVVVFSMASGRHSTE | 60 |
| 1ZB6_designed_6_a | MSEAADVERVYAAIEEAAGLLGVACARDKIWPLLSTFQDTLVEGGSVVVFSMASGRHSTE | 60 |
| 1ZB6_designed_7_a | MSEAADVERVYAAIEEAAGLLGVACARDKIWPILSTFQDTLVEGGSVVVFSMASGRHSTE | 60 |
| NPHBM31 | MSEAADVERVYAAMEEAAGLLGVACARDKIYPLLSTFQDTLVEGGSVVVFSMASGRHSTE | 60 |
| WTNPHB | MSEAADVERVYAAMEEAAGLLGVACARDKIYPLLSTFQDTLVEGGSVVVFSMASGRHSTE | 60 |
| | *************:****************:*:*************************** | |
| 1ZB6_designed_4_a | LDFSISVPTSHGDPYATVVEKGLFPATGHPVDDLLADIQKHLPVSMFAIDGEVTGGFKKT | 120 |
| 1ZB6_designed_5_a | LDFSISVPPSHGDPYAIVVEKGLFPATGHPVDDLLADIQKHLPVSMFAIDGEVTGGFKKT | 120 |
| 1ZB6_designed_6_a | LDFSISVPPSHGDPYAIVVAKGLFPATGHPVDSLLADIQKHLPVSMFAIDGEVTGGFKKT | 120 |
| 1ZB6_designed_7_a | LDFSISVPPSHGDPYAIAVAKGLFPATGHPVDSLLADIQKHLPVSMFAIDGGVVGGFKKT | 120 |
| NPHBM31 | LDFSISVPTSHGDPYATVVEKGLFPATGHPVDDLLADTQKHLPVSMFAIDGEVTGGFKKT | 120 |
| WTNPHB | LDFSISVPTSHGDPYATVVEKGLFPATGHPVDDLLADTQKHLPVSMFAIDGEVTGGFKKT | 120 |
| | ******** ******* .* ************.**** ************* *.****** | |
| 1ZB6_designed_4_a | YAFFPTDNMPGVAELAAIPSMPPAVAENAELFARYGLDKVQMTSMDYKKRQVNLYFSELS | 180 |
| 1ZB6_designed_5_a | YAFFPTDNMPGVAELAAIPSMPPAVAENAELFARYGLDKVQMTSMDYKKRQVNLYFSELS | 180 |
| 1ZB6_designed_6_a | YAFFPPDNLPQVAELAAIPSMPPAVAENAELFARYGLDKVQMTSMDYKKRQVNLYFSELS | 180 |
| 1ZB6_designed_7_a | YAFFPPDNLPQVAELAAIPSMPPAVAENAELFARYGLDKVQMTSMDYKKRQVNLYFSELS | 180 |
| NPHBM31 | YAFFPTDNMPGVAELSAIPSMPPAVAENAELFARYGLDKVQMTSMDYKKRQVNLYFSELS | 180 |
| WTNPHB | YAFFPTDNMPGVAELSAIPSMPPAVAENAELFARYGLDKVQMTSMDYKKRQVNLYFSELS | 180 |
| | ***** **:* ****:******************************************** | |
| 1ZB6_designed_4_a | AQTLEAESVLALVRELGLHVPNELGLKFCKRSFSVYPTLNWDTSKIDRLCFAVISTDPTL | 240 |
| 1ZB6_designed_5_a | AQTLEAESVLALVRELGLHVPNELGLKFCKRSFSVYPTLNWDTSKIDRLCFAVISTDPTL | 240 |
| 1ZB6_designed_6_a | AQTLEAESVLALVRELGLHVPNELGLKFCKRSFSVYPTLNWDTSKIDRLCFAVISTDPTL | 240 |
| 1ZB6_designed_7_a | AQTLEAESVLALVRELGLHVPNELGLKFCKRSFSVYPTLNWDTSQIDRLCFAVISTDPTL | 240 |
| NPHBM31 | AQTLEAESVLALVRELGLHVPNELGLKFCKRSFSVYPTLNWETGKIDRLCFSVISNDPTL | 240 |
| WTNPHB | AQTLEAESVLALVRELGLHVPNELGLKFCKRSFSVYPTLNWETGKIDRLCFAVISNDPTL | 240 |
| | *****************************************:*:*.*****:***.**** | |
| 1ZB6_designed_4_a | VPSSDEGDIEKFHNYATKAPYAYVGEKRTLVYGLTLSPKEEYYKLGAYYHITDVQRKLLK | 300 |
| 1ZB6_designed_5_a | VPSSDEGDIEKFHNYATKAPYAYVGEKRTLVYGLTLSPKEEYYKLGAYYHITDVQRKLLK | 300 |
| 1ZB6_designed_6_a | VPSSDEGDIEKFHNYATKAPYAYVGEKRTLVYGLTLTPKEEYYKLGAYYHITDVQRKLLK | 300 |
| 1ZB6_designed_7_a | VPSSDEGDIEKFHNYATKAPYAYVGEKRTLVYGLTLTPKEEYYKLGAYYHITDVQRKLLK | 300 |
| NPHBM31 | VPSSDEGDIEKFHNYATKAPYAYVGEKRTLVYGLTLSPKEEYYKLGAVYHITDVQRGLLK | 300 |
| WTNPHB | VPSSDEGDIEKFHNYATKAPYAYVGEKRTLVYGLTLSPKEEYYKLGAYYHITDVQRGLLK | 300 |
| | ************************************:********** ******** *** | |
| 1ZB6_designed_4_a | AFDSLED | 307 |
| 1ZB6_designed_5_a | AFDSLED | 307 |
| 1ZB6_designed_6_a | AFDSLED | 307 |
| 1ZB6_designed_7_a | AFDSLED | 307 |
| NPHBM31 | AFDSLED | 307 |
| WTNPHB | AFDSLED | 307 |
| | ******* | |

Recombinant methods for producing and isolating modified/mutant NphB polypeptides of the disclosure are described herein. In addition to recombinant production, the polypeptides may be produced by direct peptide synthesis using solid-phase techniques (e.g., Stewart et al. (1969) Solid-Phase Peptide Synthesis (WH Freeman Co, San Francisco); and Merrifield (1963) J. Am. Chem. Soc. 85: 2149-2154; each of which is incorporated by reference). Peptide synthesis may be performed using manual techniques or by automation. Automated synthesis may be achieved, for example, using Applied Biosystems 431A Peptide Synthesizer (Perkin Elmer, Foster City, Calif.) in accordance with the instructions provided by the manufacturer.

As used herein a non-natural amino acid refers to amino acids that do not occur in nature such as N-methyl amino acids (e.g., N-methyl L-alanine, N-methyl L-valine etc.) or alpha-methyl amino acids, beta-homo amino acids, homo-amino acids and D-amino acids. In a particular embodiment, a non-natural amino acid useful in the disclosure includes a small hydrophobic non-natural amino acid (e.g., N-methyl L-alanine, N-methyl L-valine etc.).

In addition, the disclosure provides polynucleotides encoding any of the NphB variants described herein. Due to the degeneracy of the genetic code, the actual coding sequences can vary, while still arriving at the recited polypeptide for NphB mutants and variants. It will again be readily apparent that the degeneracy of the genetic code will allow for wide variation in the percent identity between polynucleotide sequences while still encoding a particular polypeptide. Generating a polynucleotide sequence from an amino acid sequence is routine in the art.

The disclosure also provide recombinant host cells and cell free systems comprising any of the NphB variant enzymes of the disclosure. In some embodiments, the recombinant cells and cell free systems are used carry out prenylation processes.

One objective of the disclosure is to produce the precursor GPP from prenol and/or isoprenol, which can then be used to prenylate added OA with a mutant NphB of the disclosure, thereby generating CBG(V)A.

The disclosure thus provides a cell-free system comprising a plurality of enzymatic steps that converts prenol and/or isoprenol to geranyl pyrophosphate. In one embodiment, the pathway comprises an ATP regeneration module.

As depicted in FIG. 1, a pathway of the disclosure comprises four modules. The first module is the isoprenoid module which converts isoprenol or prenol to GPP. The pathway comprises a plurality of enzymatic steps. For example, in a first enzymatic reaction isoprenol is phosphorylated by an enzyme having kinase activity such as hydroxyethylthiazole kinase (ThiM; EC 2.7.1.50) to form isopentenyl monophosphate (IP). The ThiM has a polypeptide sequence as set forth in SEQ ID NO:2 or sequences that have at least 85%, 87%, 90%, 92%, 95%, 97%, or 99% identity thereto and can phosphorylate isoprenol.

In some embodiments, the hydroxyethylthiazole kinase comprises from 1 to about 20 or from 1 to about 10 amino acid modifications with respect to SEQ ID NO: 2. In some embodiments, the hydroxyethylthiazole kinase comprises from 1 to 5 amino acid modifications with respect to SEQ ID NO: 2. In some embodiments, the hydroxyethylthiazole kinase comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 amino acid modifications with respect to the amino acid sequence of SEQ ID NO: 2. In some embodiments, the hydroxyethylthiazole kinase comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, or at least 45, amino acid modifications with respect to the amino acid sequence of SEQ ID NO: 2. Amino acid modifications can be independently selected from amino acid substitutions, insertions, and deletions.

The second step of the pathway can be catalyzed by, for example, isopentenyl phosphate kinase (IPK). The IPK converts isopentenyl monophosphate to isopentenyl diphosphate (IPP). While several isopentenyl phosphate kinases are known, in some embodiments, the recombinant isopentenyl phosphate kinase comprises an amino acid sequence that is at least 70% identical to the amino acid sequence of SEQ ID NO: 59 (Methanocaldococcus jannaschii IPK) (see also SEQ ID NO:61 from M. thermoacetophila). In some embodiments, the recombinant isopentenyl phosphate kinase is 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95% 96%, 97%, 98%, 99%, or 100%, or any range between two of the foregoing values, identical to the amino acid sequence of SEQ ID NO: 59. In some embodiments, the recombinant enzyme is at least 55%, at least 60%, at least 65%, at least 70%, at least 75%, at least 76%, at least 77%, at least 70%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NO: 59. In some embodiments, the recombinant enzyme is at least 50% identical to the amino acid sequence of SEQ ID NO: 59.

In some embodiments, the isopentenyl phosphate kinases comprises from 1 to about 20 or from 1 to about 10 amino acid modifications with respect to SEQ ID NO: 59. In some embodiments, the isopentenyl phosphate kinases comprises from 1 to 5 amino acid modifications with respect to SEQ ID NO: 59. In some embodiments, the isopentenyl phosphate kinases comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 amino acid modifications with respect to the amino acid sequence of SEQ ID NO: 59. In some embodiments, the isopentenyl phosphate kinases comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, or at least 45, amino acid modifications with respect to the amino acid sequence of SEQ ID NO: 59. Amino acid modifications can be independently selected from amino acid substitutions, insertions, and deletions.

A third enzymatic step in the isoprenoid module comprises the conversion of IPP to dimethylallyl diphosphate (DMAPP) or vice-a-versa using an enzyme having isopentenyl pyrophosphate isomerase (IDI) activity. The isopentenyl pyrophosphate isomerase (IDI), can be a bacterial IDI or yeast IDI. In some embodiments, IDI isomerizes IPP to DMAPP and/or DMAPP to IPP. While several isopentenyl pyrophosphate isomerases are known, in some embodiments, the isopentenyl pyrophosphate isomerase comprises an amino acid sequence that is at least 70% identical to the amino acid sequence of SEQ ID NO: 63 (Escherichia coli IDI). In some embodiments, the isopentenyl pyrophosphate isomerase is 50%, 55%, 60%, 65%, 70%, 75%, 76%, 77%, 70%, 79%, 80%, 81%, 82%, 83%, 84%, 85%, 86%, 87%, 88%, 89%, 90%, 91%, 92%, 93%, 94%, 95% 96%, 97%, 98%, 99%, or 100%, or any range between any two of the foregoing values, identical to the amino acid sequence of SEQ ID NO: 63. In some embodiments, the isopentenyl pyrophosphate isomerase is at least 50%, at least 55%, at least 60%, at least 65%, at least 70%, at least 75%, at least 76%, at least 77%, at least 70%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NO: 63.

In some embodiments, the isopentenyl pyrophosphate isomerase comprises from 1 to about 20 or from 1 to about 10 amino acid modifications with respect to SEQ ID NO: 63. In some embodiments, the isopentenyl pyrophosphate isomerase comprises from 1 to 5 amino acid modifications with respect to SEQ ID NO: 63. In some embodiments, the isopentenyl pyrophosphate isomerase comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 amino acid modifications with respect to the amino acid sequence of SEQ ID NO: 63. In some embodiments, the isopentenyl pyrophosphate isomerase comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, or at least 45, amino acid modifications with respect to the amino acid sequence of SEQ ID NO: 63. Amino acid modifications can be independently selected from amino acid substitutions, insertions, and deletions.

In a fourth enzymatic reaction in the isoprenoid module geranyl pyrophosphate (GPP) is formed from the combination of DMAPP and isopentenyl pyrophosphate (IPP) in the presence of farnesyl-PP synthase having an S82F mutation relative to SEQ ID NO:65. In one embodiment, the farnesyl-diphosphate synthase has a sequence that is at least 95%, 98%, 99% or 100% identical to SEQ ID NO:65 having an S82F mutation and which is capable of forming geranyl pyrophosphate from DMAPP and isopentyl pyrophosphate.

In some embodiments, the farnesyl-PP synthase comprises from 1 to about 20 or from 1 to about 10 amino acid modifications with respect to SEQ ID NO: 65. In some embodiments, the farnesyl-PP synthase comprises from 1 to 5 amino acid modifications with respect to SEQ ID NO: 65. In some embodiments, the farnesyl-PP synthase comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 amino acid modifications with respect to the amino acid sequence of SEQ ID NO: 65. In some embodiments, the farnesyl-PP synthase comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, or at least 45, amino acid modifications with respect to the amino acid sequence of SEQ ID NO: 65. Amino acid modifications can be independently selected from amino acid substitutions, insertions, and deletions.

The conversion of isoprenol to GPP utilizes ATP. The pathway of FIG. 1 comprises a second module comprising an ATP regeneration module that converts acetyl phosphate and ADP to acetic acid and ATP using an acetyl kinase (AckA). In the pathway, the ATP produced by the “ATP regeneration” module can be used in the isoprenoid pathway and aromatic polyketide module. Acetate kinase is encoded in E. coli by ackA. AckA is involved in conversion of acetyl-coA to acetate. Specifically, ackA catalyzes the conversion of acetyl-phophate to acetate. AckA homologs and variants are known. The NCBI database list approximately 1450 polypeptides as bacterial acetate kinases. For example, such homologs and variants include acetate kinase (Streptomyces coelicolor A3(2)) gi|21223784|ref|NP_629563.1|(21223784); acetate kinase (Streptomyces coelicolor A3(2)) gi|6808417|emb|CAB70654.1|(6808417); acetate kinase (Streptococcus pyogenes M1 GAS) gi|15674332|ref|NP_268506.1|(15674332); acetate kinase (Campylobacter jejuni subsp. jejuni NCTC 11168) gi|15792038|ref|NP_281861.1|(15792038); acetate kinase (Streptococcus pyogenes M1 GAS) gi|13621416|gb|AAK33227.1|(13621416); acetate kinase (Rhodopirellula baltica SH 1) gi|32476009|ref|NP_869003.1|(32476009); acetate kinase (Rhodopirellula baltica SH 1) gi|32472045|ref|NP_865039.1|(32472045); acetate kinase (Campylobacter jejuni subsp. jejuni NCTC 11168) gi|112360034|emb|CAL34826.1|(112360034); acetate kinase (Rhodopirellula baltica SH 1) gi|32446553|emb|CAD76388.1|(32446553); acetate kinase (Rhodopirellula baltica SH 1) gi|32397417|emb|CAD72723.1|(32397417); AckA (Clostridium kluyveri DSM 555) gi|153954016|ref|YP_001394781.1|(153954016); acetate kinase (Bifidobacterium longum NCC2705) gi|23465540|ref|NP_696143.1|(23465540); AckA (Clostridium kluyveri DSM 555) gi|146346897|gb|EDK33433.1|(146346897); Acetate kinase (Corynebacterium diphtheriae) gi|38200875|emb|CAE50580.1|(38200875); acetate kinase (Bifidobacterium longum NCC2705) gi|23326203|g|IAAN24779.1|(23326203); Acetate kinase (Acetokinase) gi|67462089|sp|P0A6A3.1|ACKA_ECOLI(67462089); and AckA (Bacillus licheniformis DSM 13) gi|52349315|gb|AAU41949.1|(52349315), the sequences associated with such accession numbers are incorporated herein by reference.

FIG. 1 further depicts a third module, the “aromatic polyketide module”. This module generates olivetolic acid (OA). Generally, the aromatic polyketide OA or DA is derived from hexanoate (or butyrate) and malonate. Malonyl-CoA is generated from malonate via a non-natural transfer of CoA from acetyl-CoA using MdcA.

In a first enzymatic step hexanoate or butyrate is converted to hexanoyl-CoA using an acyl activating enzyme 3 (AAE3). In some embodiments, the AAE3 polypeptide comprises the amino acid sequence set forth in SEQ ID NO:4. In some embodiments, the AAE polypeptide is obtained from C. sativa. In another or further embodiment, the AAE3 polypeptide comprises an amino acid sequence having at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or 100% amino acid sequence identity to SEQ ID NO:4 (See also homologous sequences of SEQ ID NO:66-69).

In some embodiments, the acyl activating enzyme 3 (AAE3) comprises from 1 to about 20 or from 1 to about 10 amino acid modifications with respect to SEQ ID NO: 4. In some embodiments, the acyl activating enzyme 3 (AAE3) comprises from 1 to 5 amino acid modifications with respect to SEQ ID NO: 4. In some embodiments, the acyl activating enzyme 3 (AAE3) comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 amino acid modifications with respect to the amino acid sequence of SEQ ID NO: 4. In some embodiments, the acyl activating enzyme 3 (AAE3) comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, or at least 45, amino acid modifications with respect to the amino acid sequence of SEQ ID NO: 4. Amino acid modifications can be independently selected from amino acid substitutions, insertions, and deletions.

In a second enzymatic step of the polyketide module malonate and acetyl-CoA are converted to malonyl-coA using a subunit of an enzyme having malonate decarboxylase activity. In one embodiment, the malonate decarboxylase comprises the alpha subunit of malonate decarboxylase. In another or further embodiment, the malonate decarboxylase alpha subunit (MdcA) is obtained from Geobacillus sp. In another embodiment, the MdcA comprises an amino acid sequence having at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or 100% amino acid sequence identity to SEQ ID NO:6 and which is capable of transferring coA to malonate.

In some embodiments, the malonate decarboxylase alpha subunit (MdcA) comprises from 1 to about 20 or from 1 to about 10 amino acid modifications with respect to SEQ ID NO: 6. In some embodiments, the malonate decarboxylase alpha subunit (MdcA) comprises from 1 to 5 amino acid modifications with respect to SEQ ID NO: 6. In some embodiments, the malonate decarboxylase alpha subunit (MdcA) comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 amino acid modifications with respect to the amino acid sequence of SEQ ID NO: 6. In some embodiments, the malonate decarboxylase alpha subunit (MdcA) comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, or at least 45, amino acid modifications with respect to the amino acid sequence of SEQ ID NO: 6. Amino acid modifications can be independently selected from amino acid substitutions, insertions, and deletions.

The polyketide module includes a third enzymatic step that converts acetyl-phosphate and coA to acetyl-coA. The enzymatic step uses a phosphate acetyltransferase (PTA) (EC 2.3.1.8) that catalyzes the chemical reaction of acetyl-CoA+phosphate to CoA+acetyl phosphate and vice versa. Phosphate acetyltransferase is encoded in G. stearothermophilus (SEQ ID NO:8; Accession No. WP_053532564). PTA homologs and variants are known. There are approximately 1075 bacterial phosphate acetyltransferases available on NCBI. For example, such homologs and variants include phosphate acetyltransferase Pta (Rickettsia felis URRWXCal2) gi|67004021|gb|AAY60947.1|(67004021); phosphate acetyltransferase (Buchnera aphidicola str. Cc (Cinara cedri)) gi|116256910|gb|ABJ90592.1|(116256910); pta (Buchnera aphidicola str. Cc (Cinara cedri)) gi|116515056|ref|YP_802685.1|(116515056); pta (Wigglesworthia glossinidia endosymbiont of Glossina brevipalpis) gi|25166135|dbj|BAC24326.1|(25166135); Pta (Pasteurella multocida subsp. multocida str. Pm70) gi|12720993|gb|AAK02789.1|(12720993); Pta (Rhodospirillum rubrum) gi|25989720|gb|AAN75024.1|(25989720); pta (Listeria welshimeri serovar 6b str. SLCC5334) gi|116742418|emb|CAK21542.1|(116742418); Pta (Mycobacterium avium subsp. paratuberculosis K-10) gi|41398816|gb|AAS06435.1|(41398816); phosphate acetyltransferase (pta) (Borrelia burgdorferi B31) gi|15594934|ref|NP_212723.1|(15594934); phosphate acetyltransferase (pta) (Borrelia burgdorferi B31) gi|2688508|gb|AAB91518.1|(2688508); phosphate acetyltransferase (pta) (Haemophilus influenzae Rd KW20) gi|1574131|gb|AAC22857.1|(1574131); Phosphate acetyltransferase Pta (Rickettsia bellii RML369-C) gi|91206026|ref|YP_538381.1|(91206026); Phosphate acetyltransferase Pta (Rickettsia bellii RML369-C) gi|91206025|ref|YP_538380.1|(91206025); phosphate acetyltransferase pta (Mycobacterium tuberculosis F11) gi|148720131|gb|ABR04756.1|(148720131); phosphate acetyltransferase pta (Mycobacterium tuberculosis str. Haarlem) gi|134148886|gb|EBA40931.1|(134148886); phosphate acetyltransferase pta (Mycobacterium tuberculosis C) gi|124599819|gb|EAY58829.1|(124599819); Phosphate acetyltransferase Pta (Rickettsia bellii RML369-C) gi|91069570|gb|ABE05292.1|(91069570); Phosphate acetyltransferase Pta (Rickettsia bellii RML369-C) gi|91069569|gb|ABE05291.1|(91069569); phosphate acetyltransferase (pta) (Treponema pallidum subsp. pallidum str. Nichols) gi|15639088|ref|NP_218534.1|(15639088); and phosphate acetyltransferase (pta) (Treponema pallidum subsp. pallidum str. Nichols) gi|3322356|gb|AAC65090.1|(3322356), each sequence associated with the accession number is incorporated herein by reference in its entirety.

The polyketide module uses hexanoyl-CoA and malonyl-CoA as substrates in the enzymatic conversion to olivetolic acid (OA). The pathway starts with condensation of hexanoyl-CoA as the initial primer and malonyl-CoA as the extender unit by e.g., C. sativa olivetol synthase (OLS) (BAG14339.1; SEQ ID NO:10; see also SEQ ID NOs:70-73), generating 3,5,7-trioxododecanoyl-CoA. Then, C. sativa olivetolic acid cyclase (OAC) (AFN42527.1, SEQ ID NO:12 or several mutants comprising non-conservative substitutions of residues that improve the activity, see SEQ ID NO:74-75) cyclizes 3,5,7-trioxododecanoyl-CoA to olivetolic acid.

In some embodiments, the olivetol synthase (OLS) and/or olivetolic acid cyclase (OAC) comprises from 1 to about 20 or from 1 to about 10 amino acid modifications with respect to SEQ ID NO: 10 or 12, respectively. In some embodiments, the OAC and/or OLS comprises from 1 to 5 amino acid modifications with respect to SEQ ID NO: 10 or 12, respectively. In some embodiments, the OAC and/or OLS comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 amino acid modifications with respect to the amino acid sequence of SEQ ID NO: 10 or 12, respectively. In some embodiments, the OAC and/or OLS comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, or at least 45, amino acid modifications with respect to the amino acid sequence of SEQ ID NO: 10 or 12, respectively. Amino acid modifications can be independently selected from amino acid substitutions, insertions, and deletions.

GPP can be used as a substrate for a number of pathways leading to prenyl-flavanoids, geranyl-flavanoids, prenyl-stilbenoids, geranyl-stilbenoids, CBGA, CBGVA, CBDA, CBDVA, CBCA, CBCVA, THCA and THCVA (see, e.g., FIG. 1).

For example, with the NphB mutant, as described above, in hand, the ability to produce CBG(V)A from GPP and OA was performed. Nonane overlay can be used in the reactions to extract CBGA; CBGA is more soluble in water than nonane, which limits the amount of CBGA that can be extracted with a simple overlay. Thus, a flow system can be used that would capture CBGA from the nonane layer and trap it in a separate water reservoir. By implementing this flow system a lower concentration of CBGA can be maintained in the reaction vessel to mitigate enzyme precipitation.

The disclosure provides, in one embodiment, a cell free system for the production of GPP. Further the disclosure provides a cell free approach for the production of an array of pure cannabinoids and other prenylated natural products using the GPP pathway in combination with prenylating enzymes including, but not limited to, a mutant NphB by using substrates for the mutant NphB of the disclosure. The success of this method uses the engineered prenyltransferase of the disclosure (e.g., NphB mutants as described above), which was active, stable, and specific and eliminated the need for the native transmembrane prenyltransferase. The modularity and flexibility of the synthetic biochemistry platform provided herein has the benefits of a bio-based approach, but removes the complexities of satisfying living systems. For example, GPP toxicity did not factor into the design process. Moreover, OA is not taken up by yeast so the approach of adding it exogenously would not necessarily be possible in cells. Indeed, the flexibility of cell free systems can greatly facilitate the design-build-test cycles required for further optimization, additional pathway enzymes and reagent and co-factor modifications.

Turning to the overall pathway of FIG. 1, the disclosure provides a number of steps catalyzed by enzymes to covert a “substrate” to a product. In some instances a step may utilize a co-factor, but some steps do not use co-factors (e.g., NAD(P)H, ATP/ADP etc.). Table 1 provides a list of enzymes (in addition to those described above and elsewhere herein), organisms and reaction amounts used as well as accession numbers (the sequences associated with such accession numbers are incorporated herein by reference).

TABLE 1
Enzymes used in the enzymatic platform

| Enzyme | | | NCBI Accession |
|---|---|---|---|
| Abbreviation | Full Name | Source Organism | # |
| AAE3 | Acyl Activating Enzyme 3 | C. sativa | AFD33347.1 |
| MatB | Malonyl-CoA Synthetase | R. plaustris | CAE25665.1 |
| MdcA | Malonate Decarboxylase α subunit | Geobacillus sp. 44B | OQO99201.1 |
| PTA | Phosphotransacetylase | G. stearothermophilus | WP_053532564 |
| OLS | Olivetol Synthase | C. sativa | BAG14339.1 |
| OAC | Olivetolic Acid Cyclase | C. sativa | AFN42527.1 |
| ADK | Adenylate Kinase | G. thermodenitrificans | ABO65513 |
| Ppase | Pyrophosphatase | G. stearothermophilus | O05724 |
| CPK | Creatine Kinase | Rabbit Muscle | Sigma Aldrich |
| ThiM | Hydroxyethylthiazole kinase | E. coli | NP_416607 |
| IPK | Isopentenyl Kinase | M. jannaschii | WP_01069535 |
| IDI | Isopentyl diphosphate isomerase | E. coli | NP_417365 |
| FPPS S82F | Farnesyl Pyrophosphat Synthase | G. stearothermophilus | KOR95521 |
| NphB M31^{S} ** | Aromatic prenyltransferase | Streptomyces sp. CL190 | BAE00106.1 |

** The NCBI accession number reported is for the WT NphB enzyme. The NphB M31^{S} sequences are described elsewhere herein.

As described above, prenylation of olivetolate by GPP is carried out by the activity of the mutant NphB polypeptides described herein and above.

FIG. 1 depict the pathway as various “modules” (e.g., isoprenoid module, cannabinoid module, polyketide module). For example, the isoprenoid module produces the isoprenoid geranyl pyrophosphate (GPP) from isoprenol via a simplified isoprenoid pathway. The Aromatic Polyketide (AP) module converts the inputs malonate and hexanoate (or butyrate) into olivetolic acid (OA) or divarinic acid (DA). The cannabinoid module, uses products from the isoprenoid module and the polyketide module to yield cannabigerolic acid, which is then converted into the final cannabinoid by a cannabinoid synthase.

The disclosure provides an in vitro method of producing prenylated compounds and moreover, an in vitro method for producing cannabinoids and cannabinoid precursors (e.g., CBGA, CBGVA or CBGXA where ‘X’ refers to any chemical group at the 6 position of the 2,4-dihydroxybenzoic acid scaffold). In one embodiment, of the disclosure cell-free preparations can be made through, for example, three different methods. In a first embodiment, the enzymes of the pathway, as described herein, are purchased and mixed in a suitable buffer and a suitable substrate is added and incubated under conditions suitable for production of the prenylated compound or the cannabinoids or cannabinoid precursor (as the case may be). In some embodiments, the enzyme can be bound to a support or expressed in a phage display or other surface expression system and, for example, fixed in a fluid pathway corresponding to points in the metabolic pathway's cycle.

In a second embodiment, one or more polynucleotides encoding one or more enzymes of the pathway are cloned into one or more microorganism under conditions whereby the enzymes are expressed. Subsequently the cells are lysed and the lysed preparation comprising the one or more enzymes derived from the cell are combined with a suitable buffer and substrate (and one or more additional enzymes of the pathway, if necessary) to produce the prenylated compound or the cannabinoids or cannabinoid precursor. Alternatively, the enzymes can be isolated from the lysed preparations and then recombined in an appropriate buffer.

In a third embodiment, a combination of purchased enzymes and expressed enzymes are used to provide a pathway in an appropriate buffer. In one embodiment, heat stabilized polypeptide/enzymes of the pathway are cloned and expressed. In one embodiment, the enzymes of the pathway are derived from thermophilic microorganisms. The microorganisms are then lysed, the preparation heated to a temperature wherein the heat stabilized polypeptides of the pathway are active and other polypeptides (not of interest) are denatured and become inactive. The preparation thereby includes a subset of all enzymes in the microorganism and includes active heat-stable enzymes. The preparation can then be used to carry out the pathway to produce the prenylated compound or the cannabinoids or cannabinoid precursor.

For example, to construct an in vitro system, all the enzymes can be acquired commercially or purified by affinity chromatography, tested for activity, and mixed together in a properly selected reaction buffer.

An in vivo system is also contemplated using all or portions of the foregoing enzymes in a biosynthetic pathway engineered into a microorganism to obtain a recombinant microorganism.

The disclosure also provides recombinant organisms comprising metabolically engineered biosynthetic pathways that comprise a mutant nphB for the production of prenylated compounds and may further include one or more additional microorganisms expressing enzymes for the production of cannabinoids (e.g., a co-culture of one set of microorganism expressing a partial pathway and a second set of microorganism expression yet a further or final portion of the pathway etc.).

In one embodiment, the disclosure provides a recombinant microorganism comprising elevated expression of at least one target enzyme as compared to a parental microorganism or encodes an enzyme not found in the parental organism. In another or further embodiment, the microorganism comprises a reduction, disruption or knockout of at least one gene encoding an enzyme that competes with a metabolite necessary for the production of a desired metabolite or which produces an unwanted product. The recombinant microorganism expresses an enzymes that produces at least one metabolite involved in a biosynthetic pathway for the production of, for example, the prenylated compound or the cannabinoids or cannabinoid precursor. In general, the recombinant microorganisms comprises at least one recombinant metabolic pathway that comprises a target enzyme and may further include a reduction in activity or expression of an enzyme in a competitive biosynthetic pathway. The pathway acts to modify a substrate or metabolic intermediate in the production of, for example, a prenylated compound or cannabinoids or cannabinoid precursors. The target enzyme is encoded by, and expressed from, a polynucleotide derived from a suitable biological source. In some embodiments, the polynucleotide comprises a gene derived from a plant, bacterial or yeast source and recombinantly engineered into the microorganism of the disclosure. In another embodiment, the polynucleotide encoding the desired target enzyme is naturally occurring in the organism but is recombinantly engineered to be overexpressed compared to the naturally expression levels.

Culture conditions suitable for the growth and maintenance of a recombinant microorganism provided herein are known (see, e.g., “Culture of Animal Cells—A Manual of Basic Technique” by Freshney, Wiley-Liss, N.Y. (1994), Third Edition). The skilled artisan will recognize that such conditions can be modified to accommodate the requirements of each microorganism.

It is understood that a range of microorganisms can be modified to include all or part of a recombinant metabolic pathway suitable for the production of prenylated compounds or cannabinoids or cannabinoid precursors. It is also understood that various microorganisms can act as “sources” for genetic material encoding target enzymes suitable for use in a recombinant microorganism provided herein.

As previously discussed, general texts which describe molecular biological techniques useful herein, including the use of vectors, promoters and many other relevant topics, include Berger and Kimmel, Guide to Molecular Cloning Techniques, Methods in Enzymology Volume 152, (Academic Press, Inc., San Diego, Calif.) (“Berger”); Sambrook et al., Molecular Cloning—A Laboratory Manual, 2d ed., Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”), each of which is incorporated herein by reference in its entirety.

Examples of protocols sufficient to direct persons of skill through in vitro amplification methods, including the polymerase chain reaction (PCR), the ligase chain reaction (LCR), Qβ-replicase amplification and other RNA polymerase mediated techniques (e.g., NASBA), e.g., for the production of the homologous nucleic acids of the disclosure are found in Berger, Sambrook, and Ausubel, as well as in Mullis et al. (1987) U.S. Pat. No. 4,683,202; Innis et al., eds. (1990) PCR Protocols: A Guide to Methods and Applications (Academic Press Inc. San Diego, Calif.) (“Innis”); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al. (1990) Proc. Nat'l. Acad. Sci. USA 87: 1874; Lomell et al. (1989) J. Clin. Chem 35: 1826; Landegren et al. (1988) Science 241: 1077-1080; Van Brunt (1990) Biotechnology 8: 291-294; Wu and Wallace (1989) Gene 4:560; Barringer et al. (1990) Gene 89:117; and Sooknanan and Malek (1995) Biotechnology 13:563-564.

Improved methods for cloning in vitro amplified nucleic acids are described in Wallace et al., U.S. Pat. No. 5,426,039.

Improved methods for amplifying large nucleic acids by PCR are summarized in Cheng et al. (1994) Nature 369: 684-685 and the references cited therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, e.g., Ausubel, Sambrook and Berger, all supra.

The invention is illustrated in the following examples, which are provided by way of illustration and are not intended to be limiting.

### Examples

Reagents. Divarinic acid (DA) and olivetolic acid (OA) were purchased from Enamine and Toronto Research Chemicals respectively, and cannabigerolic acid (CBGA) standard was purchased from Sigma Aldrich. Co-factors were purchased from either Thermo Fisher Scientific or Sigma Aldrich. Bovine Serum Albumin (BSA), S. cerevisiae hexokinase (ScHex) and pyruvate kinase with lactate dehydrogenase (PKLDH) were purchased from Sigma Aldrich.

Cloning, expression and purification of enzymes. The genes for E. coli hydroxyethylthiazole kinase (EcThiM), R. palustris MatB, (RpMatB) and G. thermodenitrificans ADK (GtADK) were amplified from genomic DNA using HotStart Taq Mastermix (Denville) and then cloned into PCR amplified vectors using a modified Gibson method. The PCR cycle parameters were as follows: 95° C. for 3 min, 10 cycles of 95° C. for 15 sec, 63° C. for 30 sec (decrease 1° C./cycle), 72° C. for 1 min, 30 cycles of 95° C. for 15 sec, 55° C. for 30 sec, 72° C. for 1 min, followed by 72° C. for 10 min. Primers used for cloning ThiM and MatB are listed in Table 2. Mj IPK, Gs MdcA, NphB M31^{S}, CsAAE3, CsOLS and CsOAC were synthesized and cloned into the pET28(+) vector with Nde1/Xho1 restriction sites by Twist Bioscience. Expression plasmids for EcIDI, GsFPPS-S82F and GsPpase were described previously (Korman et al., Nat. Commun. 8:15526, 2017).

TABLE 2
Protein, Nucleic acid and Primer sequences

>EcThiM (SEQ ID NO: 1)
ATGCAAGTCGACCTGCTGGGTTCAGCGCAATCTGCGCACGCGTTA
CACCTTTTTCACCAACATTCCCCTCTTGTGCACTGCATGACCAAT
GATGTGGTGCAAACCTTTACCGCCAATACCTTGCTGGCGCTCGGT
GCATCGCCAGCGATGGTTATCGAAACCGAAGAGGCCAGTCAGTTT
GCGGCTATCGCCAGTGCCTTGTTGATTAACGTTGGCACACTGACG
CAGCCACGCGCTCAGGCGATGCGTGCTGCCGTTGAGCAAGCAAAA
AGCTCTCAAACACCCTGGACGCTTGATCCAGTAGCGGTGGGTGCG
CTCGATTATCGCCGCCATTTTTGTCATGAACTTTTATCTTTTAAA
CCGGCAGCGATACGTGGTAATGCTTCGGAAATCATGGCATTAGCT
GGCATTGCTAATGGCGGACGGGGAGTGGATACCACTGACGCCGCA
GCTAACGCGATACCCGCTGCACAAACACTGGCACGGGAAACTGGC
GCAATCGTCGTGGTCACTGGCGAGATGGATTATGTTACCGATGGA
CATCGTATCATTGGTATTCACGGTGGTGATCCGTTAATGACCAAA
GTGGTAGGAACTGGCTGTGCATTATCGGCGGTTGTCGCTGCCTGC
TGTGCGTTACCAGGCGATACGCTGGAAAATGTCGCATCTGCCTGT
CACTGGATGAAACAAGCCGGAGAACGCGCAGTCGCCAGAAGCGAG
GGGCCAGGCAGTTTTGTTCCACATTTCCTTGATGCGCTCTGGCAA
TTGACGCAGGAGGTGCAGGCATAA
>CsAAE3 (SEQ ID NO: 3)
ATGGGCAGCAGCCATCATCATCATCATCACAGCAGCGGCCTGGTG
CCGCGCGGCAGCCATATGGAAAAGAGTGGCTACGGACGCGACGGT
ATTTACCGTAGCCTGCGTCCTCCTTTACACCTGCCAAACAATAAC
AATTTGAGTATGGTCTCATTCCTGTTCCGTAACAGCAGCAGCTAT
CCACAGAAACCGGCGTTGATCGATAGCGAGACTAATCAAATTTTA
TCTTTTAGTCATTTTAAAAGCACCGTGATCAAGGTCTCCCATGGC
TTCTTAAACCTGGGGATCAAAAAGAATGACGTGGTTTTAATCTAC
GCACCCAATTCGATCCACTTTCCCGTATGCTTCCTTGGCATTATT
GCTTCTGGGGCGATCGCCACTACTTCAAATCCATTATACACCGTG
AGTGAGTTGTCGAAACAAGTAAAGGACTCGAACCCTAAATTGATT
ATCACAGTCCCTCAGTTATTGGAAAAGGTCAAGGGTTTCAATCTG
CCAACTATCCTTATCGGCCCTGATTCTGAGCAGGAATCGTCTAGT
GATAAAGTAATGACTTTCAATGATCTGGTCAATCTGGGAGGAAGT
TCGGGTAGCGAATTCCCTATCGTCGACGATTTCAAGCAATCCGAC
ACCGCCGCACTGTTGTACTCAAGTGGCACGACAGGTATGAGCAAG
GGGGTCGTTCTGACGCACAAAAATTTTATTGCCTCATCGTTGATG
GTAACAATGGAACAGGACTTGGTCGGCGAGATGGACAATGTGTTC
CTGTGTTTCCTTCCTATGTTTCACGTCTTTGGCTTAGCCATTATT
ACGTATGCTCAGTTACAGCGCGGTAATACCGTGATTTCAATGGCC
CGCTTTGACTTGGAAAAGATGTTAAAAGATGTTGAAAAGTACAAA
GTTACCCACCTTTGGGTCGTACCCCCAGTTATCTTAGCGTTGTCG
AAGAACTCAATGGTGAAAAAATTCAATTTGTCATCCATCAAGTAT
ATTGGTTCAGGCGCTGCGCCATTAGGAAAGGATCTGATGGAAGAA
TGCTCTAAGGTGGTTCCTTACGGAATCGTGGCTCAAGGATATGGC
ATGACGGAAACGTGCGGAATCGTATCCATGGAAGACATCCGCGGC
GGGAAACGCAATTCAGGGTCGGCCGGAATGTTGGCAAGTGGGGTA
GAAGCTCAGATCGTGAGTGTGGACACCTTAAAACCCCTTCCCCCG
AATCAATTAGGGGAAATCTGGGTAAAAGGTCCAAATATGATGCAA
GGCTATTTCAACAATCCTCAAGCGACCAAACTTACCATTGATAAA
AAGGGTTGGGTTCATACTGGCGACTTGGGGTATTTCGACGAAGAC
GGACACTTATATGTTGTAGACCGTATTAAGGAGCTTATTAAATAC
AAGGGATTCCAAGTTGCGCCTGCGGAACTGGAGGGATTATTAGTT
AGTCACCCCGAGATCTTAGACGCGGTAGTTATTCCCTTCCCCGAT
GCTGAGGCAGGCGAAGTCCCGGTGGCATACGTTGTTCGCTCGCCT
AACAGTTCGTTGACCGAAAATGACGTTAAAAAATTCATCGCCGGT
CAGGTCGCCTCCTTTAAGCGTCTGCGCAAGGTTACTTTTATTAAT
TCCGTCCCCAAGAGCGCAAGTGGGAAGATTCTGCGCCGCGAGCTT
ATTCAAAAGGTTCGCTCTAACATGTAA
>GsMdcA (SEQ ID NO: 5)
ATGGGCAGCAGCCATCATCATCATCATCACAGCAGCGGCCTGGTG
CCGCGCGGCAGCCATATGAATAGAATACACCGGTCTAAACGTTCA
TGGACAACGCGTCGCGATGCGAAGGCAAAGCGAATGGCAAAATTG
GAGCGAGTCGTGAACGGAAAAATTATACCAACAGATAAAATTGTA
GAGGCATTAGAAGCGGTTATTGCTCCAGGGGATCGTGTTGTGTTA
GAAGGAAATAATCAAAAACAAGCTTCGTTTCTATCCAAGGCATTA
TCCAAAGTTAACCCTGAGAAAGTGAACGGATTACATATGATTATG
TCCAGTGTATCGCGACCAGAGCATTTAGATATATTTGAAAAAGGA
ATCGCTAGAAAAATTGATTTTTCTTATGCCGGCCCACAAAGTCTT
CGCATGTCACAAATGCTGGAAGACGGAAAGCTTATTATAGGGGAA
ATCCATACCTATCTTGAGCTATATGGGCGGTTATTTATTGATTTG
ACTCCGTCTGTTGCACTAGTGGCGGCGGATAAAGCAGACCGATCG
GGCAATTTGTATACAGGACCTAATACAGAGGAAACTCCAACGCTT
GTTGAAGCTACGGCATTCCGGGACGGAATCGTTATAGCCCAAGTA
AATGAACTGGCAGATGAACTGCCACGGGTAGATATACCTGGCTCT
TGGATTGATTTTATCGTTGTTGCTGACCAGCCTTATGAATTAGAA
CCTCTTTTTACAAGAGATCCTCGCCTTATTACAGAAATCCAGATT
CTTATGGCGATGATGACGATTAGAGGGATATATGAACGTCATAAC
ATCCAATCTCTCAACCATGGAATCGGATTTAATACTGCGGCGATT
GAGTTATTGCTTCCAACGTACGGAGAATCATTAGGATTGAAGGGG
AAAATTTGCAGACATTGGGCATTGAATCCGCATCCTACCCTTATA
CCAGCTATTGAAACAGGATGGGTAGAAAGCATTCATTGTTTTGGA
GGAGAAGTAGGAATGGAAAAGTATATTGCGGCACGTCCCGATGTG
TTCTTTACTGGAAAAGATGGGAGTTTACGTTCAAACCGGGCATTA
TCCCAAGTAGCTGGACAGTATGCTGTCGATCTTTTTATCGGTTCT
ACTCTACAGATGGATAGGGATGGGAATTCTTCAACAGTAACGATT
GGAAGACTGGCAGGATTCGGCGGGGCACCAAACATGGGGCATGAT
CCTCGTGGACGGCGCCATTCCACTCCTGCATGGCTAGATATGATA
ACGTCCGATCATCCGATCGCGAAAGGAAAAAAATTAGTCGTGCAG
ATAGTAGAAACGTTTCAAAAAGGAAATCGACCGGTATTTGTTGAG
TCTTTAGATGCGATTGAAGTAGGGAAAAAGGCGAATTTGGCGACA
GCGCCAATTATGATATATGGGGATGATGTGACCCATGTTGTCACT
GAAGAAGGAATCGCATATTTGTATAAGGCGAATAGTTTAGAAGAA
CGCCGTCAGGCCATTGCGGCAATCGCCGGAGTCACACCGATTGGG
CTAGAACATGATCCAAAAAGAACTGAGCAGTTGCGAAGGGATGGA
TTGGTGGCGTTTCCGGAGGATTTAGGCATACGCCGTACCGATGCC
AAACGTTCTTTATTAGCAGCAAAAAGCATTGAAGAACTGGTTGAA
TGGTCGGAGGGATTGTATGAACCGCCGGCTAGATTTCGCAGCTGG
TAA
>GsPTA (SEQ ID NO: 7)
ATGGGCAGCAGCCATCATCATCATCATCACAGCAGCGGCCTGGTG
CCGCGCGGCAGCCATATGACAACCGATTTATTTACGGCATTAAAA
GCGAAAGTAACCGGTACGGCTCGAAAAATCGTGTTTCCCGAGGGA
ACCGATGACCGCATCTTAACGGCGGCGAGCCGTTTGGCGACGGAG
CAAGTGCTTCAGCCGATCGTCCTTGGCGATGAGCAAGCGATAAGG
GTGAAAGCAGCTGCGCTTGGCTTGCCGCTTGAAGGGGTGGAGATT
GTCAACCCGCGCCGCTACGGCGGGTTTGATGAGCTAGTTTCGGCG
TTTGTGGAGCGGCGCAAAGGGAAAGTGACAGAAGAAACGGCGCGC
GAGTTGCTTTTCGATGAAAACTATTTCGGTACGATGCTCGTTTAT
ATGGGAGCGGCCGACGGCCTCGTCAGCGGGGCGGCACATTCGACG
GCGGATACGGTCCGACCAGCCTTGCAAATCATTAAAACGAAGCCA
GGCGTTGACAAAACGTCCGGCGTGTTCATCATGGTGCGCGGCGAC
GAAAAATATGTGTTTGCCGATTGCGCCATCAACATTGCTCCTAAC
AGTCATGATTTGGCTGAAATCGCGGTCGAGAGCGCCCGGACGGCC
AAAATGTTCGGCCTTAAGCCGCGCGTAGTGCTGTTAAGCTTTTCC
ACGAAAGGGTCGGCCTCGTCGCCGGAGACGGAAAAAGTCGTTGAG
GCGGTGCGGTTGGCGAAAGAAATGGCGCCGGATCTGATCCTTGAC
GGTGAGTTTCAATTTGACGCCGCGTTTGTGCCAGAGGTGGCGAAA
AAGAAAGCGCCGGACTCGGTCATTCAAGGGGACGCAAATGTCTTT
ATTTTCCCGAGCCTTGAGGCGGGCAACATCGGCTACAAAATCGCC
CAGCGCCTTGGCGGCTTTGAAGCGGTTGGCCCGATTTTGCAAGGG
CTGAACAAGCCGGTTAACGACCTATCGCGCGGCTGCAGCGCCGAA
GACGCCTACAAGCTCGCGCTCATCACCGCGGCGCAGTCGCTTGGG
GAG
>CSOLS (SEQ ID NO: 9)
ATGGGCAGCAGCCATCATCATCATCATCACAGCAGCGGCCTGGTG
CCGCGCGGCAGCCATATGAATCATCTGCGTGCTGAAGGACCAGCT
TCCGTATTGGCAATTGGAACAGCTAACCCTGAGAACATTCTTCTT
CAGGATGAGTTTCCCGACTATTACTTCCGCGTGACAAAGAGCGAA
CACATGACACAGCTTAAAGAGAAGTTCCGTAAGATCTGTGACAAA
AGCATGATCCGCAAACGTAACTGCTTCCTTAACGAGGAGCATCTG
AAGCAGAATCCCCGTCTTGTTGAACATGAGATGCAGACCTTGGAT
GCTCGCCAGGACATGTTGGTTGTTGAGGTCCCTAAGCTGGGCAAA
GATGCGTGTGCAAAAGCGATTAAAGAGTGGGGGCAGCCTAAAAGC
AAAATTACTCATCTGATTTTCACAAGCGCCAGTACAACCGATATG
CCCGGTGCGGACTACCATTGTGCAAAATTATTGGGTTTATCGCCT
TCAGTAAAACGTGTTATGATGTACCAGTTAGGATGCTACGGTGGT
GGCACCGTACTTCGTATTGCGAAGGACATCGCCGAGAACAACAAA
GGAGCCCGTGTACTTGCTGTATGTTGTGATATCATGGCGTGCCTT
TTTCGCGGCCCCAGCGAGAGTGACCTTGAGTTACTTGTGGGGCAG
GCCATCTTCGGAGACGGTGCCGCAGCCGTCATTGTTGGCGCAGAG
CCCGATGAATCCGTTGGCGAGCGCCCGATCTTTGAGCTTGTAAGT
ACAGGACAAACTATCTTGCCCAACTCTGAGGGGACTATCGGCGGA
CATATTCGTGAGGCGGGCTTGATTTTTGACCTTCACAAGGATGTT
CCAATGCTTATCTCCAATAATATTGAAAAATGTCTTATCGAAGCA
TTCACTCCGATTGGTATCTCCGATTGGAATTCGATTTTTTGGATC
ACCCATCCTGGTGGGAAAGCTATTTTAGACAAGGTGGAGGAGAAA
TTACATCTTAAGTCAGATAAGTTTGTCGACAGTCGCCACGTGTTG
TCGGAACATGGCAACATGTCATCGTCAACCGTCTTGTTCGTTATG
GACGAATTACGTAAACGCAGTTTAGAAGAGGGTAAGAGTACGACG
GGGGACGGGTTCGAGTGGGGAGTCTTATTCGGGTTCGGTCCAGGA
TTGACAGTGGAACGCGTCGTGGTTCGCAGTGTCCCCATTAAGTAC
TAA
>CSOAC (SEQ ID NO: 11)
ATGGGCAGCAGCCATCATCATCATCATCACAGCAGCGGCCTGGTG
CCGCGCGGCAGCCATATGGCAGTCAAACACTTGATCGTGTTAAAG
TTCAAAGATGAAATCACAGAGGCTCAGAAGGAAGAATTTTTCAAG
ACGTATGTAAACCTTGTTAATATCATCCCCGCTATGAAGGATGTG
TATTGGGGTAAAGACGTGACACAGAAGAACAAAGAGGAAGGCTAC
ACGCACATCGTAGAGGTCACATTTGAGAGCGTCGAAACTATTCAG
GATTACATCATTCATCCCGCACACGTTGGATTCGGGGATGTGTAT
CGCTCTTTCTGGGAAAAATTGCTGATCTTCGACTATACACCGCGT
AAGTAA
>GtADK (SEQ ID NO: 13)
ATGAATTTAGTGCTGATGGGGCTGCCAGGTGCCGGCAAAGGCACG
CAAGCCGAGAAAATCGTAGAAACGTATGGAATCCCACATATTTCA
ACCGGGGATATGTTTCGGGCGGCGATGAAAGAAGGCACACCGTTA
GGATTGCAGGCAAAAGAATATATCGACCGTGGTGATCTTGTTCCG
GATGAGGTGACGATCGGTATCGTCCGTGAACGGTTAAGCAAAGAC
GACTGCCAAAACGGCTTTTTGCTTGACGGATTCCCACGCACGGTT
GCCCAAGCGGAGGCGCTGGAAGCGATGCTGGCTGAAATCGGCCGC
AAGCTTGACTATGTCATCCATATCGATGTTCGCCAAGATGTGTTA
ATGGAGCGCCTCACAGGCAGACGAATTTGTCGCAACTGCGGAGCG
ACATACCATCTTGTTTTTCACCCACCGGCTCAGCCAGGCGTATGT
GATAAATGCGGTGGCGAGCTTTATCAGCGCCCTGACGATAATGAA
GCAACAGTGGCGAATCGGCTTGAGGTGAATACGAAACAAATGAAG
CCATTGCTCGATTTCTATGAGCAAAAAGGCTATTTGCGCCACATT
AACGGCGAACAAGAAATGGAAAAAGTGTTTAGCGACATTCGCGAA
TTGCTCGGGGGACTTACTCGATGA
>RpMatB (SEQ ID NO: 15)
ATGAACGCCAACCTGTTCGCCCGCCTGTTCGATAAGCTCGACGAC
CCCCACAAGCTCGCGATCGAAACCGCGGCCGGGGACAAGATCAGC
TACGCCGAGCTGGTGGCGCGGGCGGGCCGCGTCGCCAACGTGCTG
GTGGCACGCGGCCTGCAGGTCGGCGACCGCGTTGCGGCGCAAACC
GAGAAGTCGGTGGAAGCGCTGGTGCTGTATCTCGCCACGGTGCGG
GCCGGCGGCGTGTATCTGCCGCTCAACACCGCCTATACGCTGCAC
GAGCTCGATTACTTCATCACCGATGCCGAGCCGAAGATCGTGGTG
TGCGATCCGTCCAAGCGCGACGGGATCGCGGCGATTGCCGCCAAG
GTCGGCGCCACGGTGGAGACGCTTGGCCCCGACGGTCGGGGCTCG
CTCACCGATGCGGCAGCTGGAGCCAGCGAGGCGTTCGCCACGATC
GACCGCGGCGCCGATGATCTGGCGGCGATCCTCTACACCTCAGGG
ACGACCGGCCGCTCCAAGGGCGCGATGCTCAGCCACGACAATTTG
GCGTCGAACTCGCTGACGCTGGTCGATTACTGGCGCTTCACGCCG
GATGACGTGCTGATCCACGCGCTGCCGATCTATCACACCCATGGA
TTGTTCGTGGCCAGCAACGTCACGCTGTTCGCGCGCGGATCGATG
ATCTTCCTGCCGAAGTTCGATCCCGACAAGATCCTCGACCTGATG
GCGCGCGCCACCGTGCTGATGGGTGTGCCGACGTTCTACACGCGG
CTCTTGCAGAGCCCGCGGCTGACCAAGGAGACGACGGGCCACATG
AGGCTGTTCATCTCCGGGTCGGCGCCGCTGCTCGCCGATACGCAT
CGCGAATGGTCGGCGAAGACCGGTCACGCCGTGCTCGAGCGCTAC
GGCATGACCGAGACCAACATGAACACCTCGAACCCGTATGACGGC
GACCGCGTCCCCGGCGCGGTCGGCCCGGCGCTGCCCGGCGTTTCG
GCGCGCGTGACCGATCCGGAAACCGGCAAGGAACTGCCGCGCGGC
GACATCGGGATGATCGAGGTGAAGGGCCCGAACGTGTTCAAGGGC
TACTGGCGGATGCCGGAGAAGACCAAGTCTGAATTCCGCGACGAC
GGCTTCTTCATCACCGGCGACCTCGGCAAGATCGACGAGCGCGGC
TACGTCCACATCCTCGGCCGCGGCAAGGATCTGGTGATCACCGGC
GGCTTCAACGTCTATCCGAAGGAAATCGAGAGCGAGATCGACGCC
ATGCCGGGCGTGGTCGAATCCGCGGTGATCGGCGTGCCGCACGCC
GATTTCGGCGAGGGCGTCACTGCCGTGGTGGTGCGCGACAAGGGT
GCCACGATCGACGAAGCGCAGGTGCTGCACGGCCTCGACGGTCAG
CTCGCCAAGTTCAAGATGCCGAAGAAAGTGATCTTCGTCGACGAC
CTGCCGCGCAACACCATGGGCAAGGTCCAGAAGAACGTCCTGCGC
GAGACCTACAAGGACATCTACAAGTAA
>Gs PPase (SEQ ID NO: 17)
ATGGGCAGCAGCCATCATCATCATCATCACAGCAGCGGCCTGGTG
CCGCGCGGCAGCCATATGGCCTTTGAGAATAAGATTGTCGAAGCG
TTTATCGAAATTCCAACCGGCAGCCAAAACAAATACGAGTTCGAC
AAAGAGCGGGGCGTTTTCAAACTCGACCGCGTCTTGTACTCCCCG
ATGTTTTACCCGGCTGAGTACGGCTACTTGCAAAATACGCTGGCG
CTCGATGGCGACCCGCTCGACATTTTGGTCATCACAACGAATCCG
ACATTCCCGGGCTGCGTCATCGATACGCGTGTCATCGGCTTTTTG
AACATGGTCGACAGCGGTGAGGAGGACGCGAAGCTCATCGGCGTG
CCAGTCGAAGACCCGCGCTTTGATGAAGTCCGCTCGATTGAAGAC
CTGCCGCAGCACAAGCTGAAAGAAATCGCCCACTTCTTTGAACGG
TACAAAGACTTGCAAGGCAAGCGGACGGAAATCGGCACATGGGAA
GGGCCGGAAGCTGCGGCAAAACTGATCGATGAGTGCATCGCCCGC
TATAACGAACAAAAATAA
>GsFPPS-S82F (SEQ ID NO: 19)
ATGGGCAGCAGCCATCATCATCATCATCACAGCAGCGGCCTGGTG
CCGCGCGGCAGCCATATGGCGCAGCTTTCAGTTGAACAGTTTCTC
AACGAGCAAAAACAGGCGGTGGAAACAGCGCTCTCCCGTTATATA
GAGCGCTTAGAAGGGCCGGCGAAGCTGAAAAAGGCGATGGCGTAC
TCATTGGAGGCCGGCGGCAAACGAATCCGTCCGTTGCTGCTTCTG
TCCACCGTTCGGGCGCTCGGCAAAGACCCGGCGGTCGGATTGCCC
GTCGCCTGCGCGATTGAAATGATCCATACGTACTTTTTGATCCAT
GATGATTTGCCGAGCATGGACAACGATGATTTGCGGCGCGGCAAG
CCGACGAACCATAAAGTGTTCGGCGAGGCGATGGCCATCTTGGCG
GGGGACGGGTTGTTGACGTACGCGTTTCAATTGATCACCGAAATC
GACGATGAGCGCATCCCTCCTTCCGTCCGGCTTCGGCTCATCGAA
CGGCTGGCGAAAGCGGCCGGTCCGGAAGGGATGGTCGCCGGTCAG
GCAGCCGATATGGAAGGAGAGGGGAAAACGCTGACGCTTTCGGAG
CTCGAATACATTCATCGGCATAAAACCGGGAAAATGCTGCAATAC
AGCGTGCACGCCGGCGCCTTGATCGGCGGCGCTGATGCCCGGCAA
ACGCGGGAGCTTGACGAATTCGCCGCCCATCTAGGCCTTGCCTTT
CAAATTCGCGATGATATTCTCGATATTGAAGGGGCAGAAGAAAAA
ATCGGCAAGCCGGTCGGCAGCGACCAAAGCAACAACAAAGCGACG
TATCCAGCGTTGCTGTCGCTTGCCGGCGCGAAGGAAAAGTTGGCG
TTCCATATCGAGGCGGCGCAGCGCCATTTACGGAACGCTGACGTT
GACGGCGCCGCGCTCGCCTATATTTGCGAACTGGTCGCCGCCCGC
GACCATTAA
>ECIDI (SEQ ID NO: 20)
ATGCAAACGGAACACGTCATTTTATTGAATGCACAGGGAGTTCCC
ACGGGTACGCTGGAAAAGTATGCCGCACACACGGCAGACACCCGC
TTACATCTCGCGTTCTCCAGTTGGCTGTTTAATGCCAAAGGACAA
TTATTAGTTACCCGCCGCGCACTGAGCAAAAAAGCATGGCCTGGC
GTGTGGACTAACTCGGTTTGTGGGCACCCACAACTGGGAGAAAGC
AACGAAGACGCAGTGATCCGCCGTTGCCGTTATGAGCTTGGCGTG
GAAATTACGCCTCCTGAATCTATCTATCCTGACTTTCGCTACCGC
GCCACCGATCCGAGTGGCATTGTGGAAAATGAAGTGTGTCCGGTA
TTTGCCGCACGCACCACTAGTGCGTTACAGATCAATGATGATGAA
GTGATGGATTATCAATGGTGTGATTTAGCAGATGTATTACACGGT
ATTGATGCCACGCCGTGGGCGTTCAGTCCGTGGATGGTGATGCAG
GCGACAAATCGCGAAGCCAGAAAACGATTATCTGCATTTACCCAG
CTTAAACTCGAGCACCACCACCACCACCACTGA
>NphBM31^{S} (SEQ ID NO: 35)
ATGGGCAGCAGCCATCATCATCATCATCACAGCAGCGGCCTGGTG
CCGCGCGGCAGCCATATGTCGGAAGCTGCCGATGTAGAACGTGTC
TACGCCGCCATCGAAGAAGCCGCAGGTTTGTTGGGGGTCGCATGC
GCACGCGATAAGATTTGGCCCTTGCTGTCAACATTCCAGGATACC
TTGGTTGAGGGTGGAAGCGTAGTTGTTTTTAGCATGGCCTCGGGG
CGTCACTCAACGGAGCTGGACTTCTCAATTTCCGTCCCGCCTAGT
CATGGCGATCCGTACGCGATTGTGGTGGAAAAGGGCTTGTTCCCG
GCAACTGGACATCCAGTTGATGACCTTCTGGCGGACATTCAGAAG
CATCTTCCCGTATCTATGTTTGCGATTGACGGGGAAGTTACCGGG
GGGTTCAAAAAAACTTATGCGTTCTTCCCGACCGATAACATGCCC
GGTGTCGCGGAACTGGCGGCCATCCCATCGATGCCTCCTGCAGTC
GCTGAAAATGCTGAACTGTTCGCGCGTTATGGCCTGGACAAGGTA
CAAATGACCTCGATGGATTATAAAAAACGTCAAGTGAACCTGTAT
TTCTCCGAACTGTCGGCTCAGACGCTGGAGGCTGAATCAGTACTT
GCTTTAGTGCGTGAACTGGGTCTTCATGTCCCAAACGAGCTGGGT
CTGAAATTTTGCAAACGCTCCTTCTCAGTATACCCAACATTAAAC
TGGGACACCTCGAAGATTGACCGCCTTTGCTTCTCTGTAATCAGT
ACAGATCCGACACTTGTACCTAGCTCAGACGAGGGAGACATTGAA
AAATTTCACAATTACGCTACAAAGGCCCCCTATGCATATGTTGGA
GAAAAGCGTACACTTGTTTACGGCTTGACTTTATCTCCCAAAGAG
GAGTATTATAAATTGGGTGCCGTTTACCACATTACTGACGTACAA
CGCAAACTTTTGAAGGCGTTCGACAGCCTTGAGGATTAA
>Methanocaldococcus jannaschii IPK
(SEQ ID NO: 58)
ATGTTGACTATTCTTAAGTTGGGAGGGAGCATTCTGTCCGATAAA
AACGTTCCATATAGCATTAAGTGGGATAACTTAGAACGTATTGCT
ATGGAAATCAAAAACGCGTTAGATTATTACAAGAACCAAAATAAA
GAAATTAAGCTTATTCTGGTACATGGCGGCGGGGCATTTGGGCAT
CCAGTGGCCAAGAAATACCTGAAGATTGAAGACGGCAAAAAAATT
TTCATCAACATGGAAAAAGGATTCTGGGAGATTCAGCGTGCGATG
CGCCGTTTTAATAACATCATCATCGACACGCTTCAGAGTTACGAT
ATCCCAGCGGTCTCGATTCAACCTTCCAGCTTTGTTGTTTTTGGC
GACAAATTGATCTTCGACACCTCTGCGATCAAAGAGATGTTGAAA
CGCAACCTTGTACCCGTTATCCATGGGGATATCGTCATTGACGAT
AAAAATGGGTACCGTATTATCAGCGGTGACGACATCGTGCCATAT
TTAGCCAATGAACTGAAGGCAGATTTAATCCTTTATGCAACCGAC
GTGGACGGCGTATTGATTGACAACAAGCCCATTAAACGCATTGAT
AAGAATAATATCTACAAGATTTTGAATTATCTTTCGGGTAGCAAT
TCAATTGACGTCACGGGGGGGATGAAATACAAGATCGACATGATC
CGTAAAAACAAATGCCGTGGTTTCGTGTTTAATGGCAACAAGGCA
AACAACATTTATAAGGCGCTGCTTGGGGAAGTCGAGGGTACCGAA
ATCGACTTTTCTGAATAA
Primer sequences
EcThiM
FOR
5' CCGCGCGGCAGCCATATGCAAGTCGACCTGCTGGGTTCAGC
GCAATCTGC 3' (SEQ ID NO: 28)
REV
5' GGTGGTGGTGGTGGTGCTCGAGTTATGCCTGCACCTCCTGCG
TCAATTGCCAGAGCGC 3'
(SEQ ID NO: 29)
RpMatB
FOR
5' CCGCGCGGCAGCCATATGAACGCCAACCTGTTCGCCCGCCTG
TTCG 3' (SEQ ID NO: 31)
REV
5' GGTGGTGGTGGTGGTGCTCGAGTTACTTGTAGATGTCCTTGT
AGGTCTCGCGCAGG 3' (SEQ ID NO: 32)
GtADK
FOR
5' GGTGCCGCGCGGCAGCCATATGAATTTAGTGCTGATGGGGCT
GCC 3' (SEQ ID NO: 33)
REV
5' CAGTGGTGGTGGTGGTGGTGCTCGAGTTATCGAGTAAGTCCC
CCGAGC 3' (SEQ ID NO: 34)

The majority of the enzymes were expressed in E. coli BL21 (DE3) Gold, with the exception of CsOLS, CsAAE3 and GsMdcA which were expressed in the E. coli C43 BL21 (DE3). 1 L of LB media with 50 ug/mL kanamycin was inoculated with 1 mL of saturated culture, and grown to an OD₆₀₀ of 0.6-0.8. Protein expression was induced by adding IPTG to 1 mM, and the cultures were incubated overnight at 18° C. The cells were harvested by centrifugation at 2,500×g, and resuspended in 20 mL of binding buffer (50 mM Tris pH 8.0, 150 mM NaCl and 10 mM imidazole). The cells were lysed using an Emulsiflex (Avestin) instrument, and the lysate was clarified by centrifugation at 20,000×g for 20 min. A 50% v/v suspension of NiNTA resin in 20% ethanol was added to the clarified lysate (2 mL/1 L culture), and incubated with gentle mixing at 4° C. for 30 minutes. The clarified lysate was transferred to a gravity flow column. The flow through was discarded, and the column was washed with 5-10 column volumes of binding buffer. The wash was discarded, and the enzyme was eluted with 2-3 column volumes of elution buffer (50 mM Tris pH 8.0, 150 mM NaCl, 250 mM imidazole, 25% (v/v) glycerol).

Due to high ATPase activity, CsAAE3, CsOLS, CsOAC and EcThiM were purified further using size exclusion chromatography. CsAAE3, CsOLS and EcThiM were loaded (3-6 mL) onto a 16/600 Superdex 200 column. The flow rate was 1 mL/min, and the buffer was 50 mM Tris pH 8.0 and 200 mM NaCl. 2 mL elution fractions were concentrated using a 10 kDa Amicon filter from Millipore Sigma, and 15% glycerol was added. OAC was loaded (3-6 mL) onto a 16/600 Superdex 75 column. The flow rate was 1 mL/min and the buffer was 50 mM Tris pH 8.0, 200 mM NaCl and 10% glycerol. OAC precipitates without 20% glycerol, so 2 mL of 50 mM Tris pH 8, 200 mM NaCl and 40% glycerol were added to the fraction collection tubes to adjust the final glycerol concentration to 20%. OAC was then concentrated using a 5 kDa Amicon filter. The EcThiM ATPase activity was still present after SEC purification, so the elution fraction was diluted 3-fold into 50 mM Tris, and it was loaded onto a 5 mL Q sepharose column equilibrated in 50 mM Tris pH 8.0 and 50 mM NaCl. The column was washed with 50 mM Tris pH 8.0 and 50 mM NaCl, and then eluted with a linear gradient to 100% 50 mM Tris pH 8.0 1 M NaCl. Fractions containing ThiM were concentrated, and glycerol was added to 15%. All enzymes were stored a −80° C. until needed.

OA/DA Reaction Conditions using MatB. The conditions for reactions using RpMatB to produce malonyl-CoA were as follows: 15 mM malonate, 5 mM hexanoate or 5 mM butyrate, 1 mM CoA, 4 mM ATP, 25 mM creatine phosphate, 10 mM KCl, 5 mM MgCl₂ and 50 mM Tris pH 8.0, 1.3 μM RpMatB, 4.9 μM CsAAE3, 2.9 μM CsOLS, 46.6 μM CsOAC, 7.6 μM GsPpase, 2.6 μM ADK and 2 units of CPK (from Sigma Aldrich). For the additive reactions GPP (0.5-2 mM), OA (0.25-2 mM) and DA (0.25-5 mM) were added before the reaction was initiated.

For the time course, the reactions were quenched (see below) at various time points between 5 mins and 5 hours. The reactions with additives were quenched at 4 hours.

OA/DA Reaction Conditions using MdcA. The reaction conditions for experiments using the MdcA path were as follows: 4 mM ATP, 1 mM CoA, 5 mM MgCl₂, 10 mM KCl, 5 mM hexanoate or butyrate, 15 mM malonate, 50 mM acetyl phosphate, 50 mM Tris pH 8.0, 1.3 μM SeAckA, 1.4 μM GsMdcA, 4.5 μM CsAAE3, 2.9 μM CsOLS, 50 μM CsOAC, 2.6 μM GtADK, 2.6 μM GsPpase, 1.6 μM GsPTA. The effect of BSA was tested by titrating BSA into the reactions. The time course reactions contained either 20 mg/mL BSA or no BSA. The BSA titration reactions were quenched at 4 hours. The time course experiments were quenched at various time points between 0.5 and 5 hours.

Isoprenoid Reaction Conditions. The reaction conditions that tested the ability of the isoprenol pathway to generate GPP, were as follows: 1 mM ATP, 5 mM MgCl₂, 5 mM OA or DA, 50 mM acetyl phosphate, 50 mM Tris pH 8.0, 15.2 μM EcThiM, 2.1 μM MjIPK, 6.6 μM EcIDI, 2.5 μM GsFPPS-S82F, 13.2 μM NphB M31^{S}, 1.3 μM SeAckA and 20 mg/mL BSA. The reactions were quenched at various time points ranging from 0.5-25 hours.

Full pathway Reaction Conditions. The reaction conditions for the full pathway were as follows: 4 mM ATP, 1 mM CoA, 5 mM MgCl₂, 10 mM KCl, 5 mM hexanoate or butyrate, 15 mM malonate, 50 mM acetyl phosphate, 50 mM Tris pH 8.0, 1.3 μM SeAckA, 1.4 μM GsMdcA, 4.5 μM CsAAE3, 2.9 μM CsOLS, 50 μM CsOAC, 2.6 μM GtADK, 2.6 μM GsPpase, 1.6 μM GsPTA, 5.2 μM EcThiM, 2.1 μM MjIPK, 6.6 μM EcIDI, 2.5 μM GsFPPS-S82F, 13.2 μM NphB M31_{S} and 20 mg/mL BSA.

To test the effects of additives on product titer, acetate (25-100 mM) or phosphate (25-100 mM) was added before the reaction was initiated. The reaction was quenched at 6 hours. For the time course the reactions were quenched at various time points between 0.5 and 10 hours. AcP was also titrated from 25 mM to 200 mM to ensure the optimal starting conditions were being used. Those reactions were quenched at 4 hours.

Recycled Enzyme Reaction Conditions. The reaction conditions were identical to those detailed above under full pathway reaction conditions. At 6 hours 200 μL of the reaction mixture was added to a 3 kDa protein concentrator, and 300 μL of buffer (50 mM Tris pH 8.0 and 200 mM NaCl) was added. The sample volume was reduced to 100 μL after 15 minutes of centrifugation at 16,000×g at 4 C. Then, 400 μL of buffer (50 mM Tris pH 8.0 and 200 mM NaCl) was added to the protein concentrator, and centrifuged for another 15 mins at 16,000×g at 4 C. Then a new reaction was set up as follows: 100 μL of enzymes from the protein concentrator, 4 mM ATP, 1 mM CoA, 5 mM MgCl₂, 10 mM KCl, 5 mM hexanoate, 15 mM malonate, 50 mM acetyl phosphate and 50 mM Tris pH 8.0. The secondary reaction was quenched after an additional 31 hours (37 total).

HPLC Sample Analysis. All samples were quenched by 4-fold dilution into methanol (samples with a higher concentration of analyte were diluted up to 10-fold). The protein precipitate was removed by centrifugation at 16,000×g for 5 minutes and the supernatant was transferred to an LC vial for analysis.

Samples were analyzed by reverse phase chromatography on a Syncronis C8 column (4.6×100 mm) using a Thermo Ultimate 3000 HPLC. The column compartment temperature was set to 40° C., and the flow rate was 1 mL/min. The sample inject volume was 20 μL (full loop). The compounds were separated using a gradient elution with water+0.1% TFA (solvent A) and acetonitrile+0.1% TFA (solvent B) as the mobile phase. Solvent B was held at 20% for the first minute. Then solvent B was increased to 95% B over 4 minutes, and held at 95% B for 3 minutes. The column was then re-equilibrated to 20% B for three minutes, for a total run time of 11 minutes. Standards were used to identify the retention time, and to produce an external standard curve for quantification.

GPP Quantification Assay. A 50 μL aliquot of the reaction was quenched in 150 μL of methanol. The proteins were removed by centrifugation, and the supernatant was dried using a speed vac. Once the solvent was removed, 50 μL of Tris pH 8.0 and 2 units of calf intestinal alkaline phosphatase (CIP) were added. The reaction was incubated for 16 hours, and the reaction was extracted with 100 μL of hexane. The reaction extract was analyzed on a Thermo Scientific Trace 1310 GC-FID instrument equipped with a Thermo Scientific TG-WAXMS column (30m x 0.32 mm x 0.25 μM). The carrier gas was helium (30 mL/min), the split ratio was 1:1, the inject volume was 2 μL and the inlet temperature was set to 250° C. The initial temperature was held at 80° C. for 6 minutes, increased to 260° C. at a rate of 12° C./min, and held at 260° C. for 3 minutes, for a total run time of 24 minutes. GPP was quantified based on an external standard curve that was prepared in the same manner as the samples.

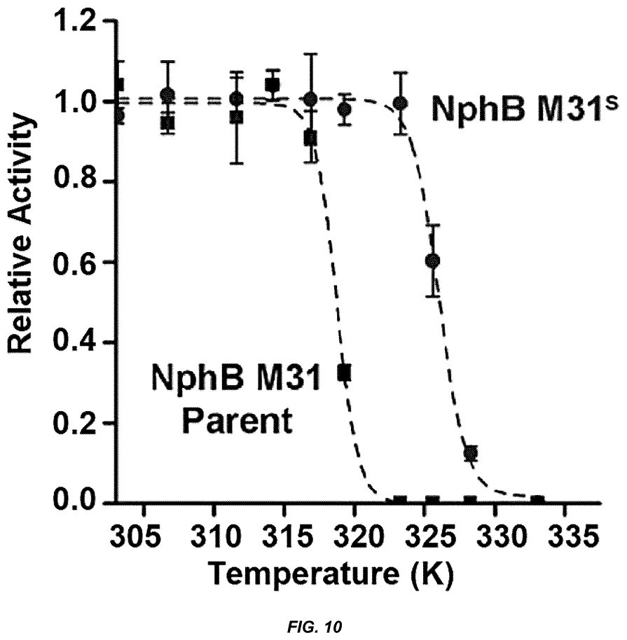

Stabilization of NphB. A stabilized version of the previously described NphBM31 enzyme was developed, using the PROSS software with default parameters. Chain A of the crystal structure of the wild-type Orf2 from Streptomyces sp. CL190 (RCSB:1ZB6) was used as the starting model. Small molecule ligands Mg²⁺ (MG) and 1,6 dihydroxynaphthalene (DHN) were input to exclude mutations to the active site. A mutant designated NphB M31S with the following mutations was found to stabilize the enzyme to thermal inactivation: M14I, Y31W, T69P, T77I, T98I, S136A, E222D, G224S, A232S, N236T, Y288V and G297K. The thermal inactivation profile of NphB M31 and NphB M31^{S} are compared in FIG. 10. To obtain the thermal inactivation profile either 1 mg/ml NphB M31 Parent NphB M31^{S} were heated for 20 minutes at 303.1, 306.7, 311.6, 314.2, 316.9, 319.3, 323.3, 325.6 328.3 and 333.1 K in an Eppendorf thermocycler and assayed for remaining activity.

ATPase Assay. To measure the amount of ATPase activity added to the reactions, ATPase activity was coupled to PKLDH. The reaction conditions were as follows: 5 mM PEP, 2 mM ATP, 1 mM NADH, 5 mM MgCl₂, 10 mM KCl, ˜1 U PKLDH (Sigma) and the enzyme master mix from the Full Pathway Reaction Conditions. The decrease in NADH absorbance at 340 nm was used as a measure of background ATPase activity.

MatB Activity Assay. A coupled enzymatic assay was used to determine the activity of MatB in the presence of OA and DA. The reaction conditions were: 2.5 mM malonate, 2 mM ATP, 1 mM CoA, 2.5 mM phosphoenolpyruvate (PEP), 1 mM NADH, 5 mM MgCl₂, 10 mM KCl, 0.35 mg/mL ADK, 0.75 μg/mL MatB, 1.6 units of PK and 2.5 units of LDH, and 50 mM Tris [pH 8.0]. Background ATPase activity was controlled for by leaving out the substrate (malonate), and either 1% ethanol, 250 μM or 5 mM OA or 5 mM DA was added to the remaining reactions. The activity of MatB was determined by monitoring decreasing absorbance at 340 nm due to NADH consumption using an M2 SpectraMax. To ensure that MatB was limiting at 5 mM OA or DA, MatB was doubled to 1.5 μg/mL. The rate of the reaction doubled indicating that MatB was the limiting component in the system. The rate of NADH consumption at 5 mM OA and 5 mM DA was normalized to the 1% ethanol control.

AAE3 Activity Assay. A coupled enzymatic assay, similar to the one above was used to determine the activity of AAE3 in the presence of OA and DA. The conditions were the same as the MatB assay with the following modifications: 2.5 mM hexanoate was added in lieu of malonate, and 15 μg/mL of AAE3 was added in lieu of MatB. To ensure that AAE3 was limiting, AAE3 was doubled in the presence of 5 mM OA or DA. The rate of the reaction doubled indicating AAE3 was limiting.

CPK Activity Assay. A coupled enzymatic assay was used to determine the activity of CPK in the presence of OA or DA. The reaction conditions were: 5 mM Creatine Phosphate, 2 mM ADP, 5 mM glucose, 2 mM NADP⁺, 5 mM MgCl₂, 5 mM KCl, 0.3 mg/mL Zwf, 0.1 mg/mL Sc Hex and 0.08 units CPK. The positive control reaction contained 1% ethanol, and either 5 mM of OA or DA was added to the remaining reactions. The absorbance of NADPH at 340 nm was monitored. To ensure that CPK was limiting, the CPK addition was doubled at 5 mM OA and 5 mM DA. The resulting rate doubled, which indicates CPK is limiting even at high OA and DA.

ADK Activity Assay. A coupled enzymatic assay was used to determine the activity of ADK in the presence of OA and DA. The conditions were similar to the MatB assay, with the following modifications: 2 mM AMP was added in lieu of malonate, CoA was not added, and 0.001 mg/mL of ADK was added. To ensure that ADK was the limiting reagent at 5 mM OA and DA, the amount of ADK was doubled. The 2-fold increase in rate indicated that ADK was the limiting factor.

OLS Activity Assay. For the inhibition experiments the conditions were altered to: 1 mM malonyl-CoA, 400 μM hexanoyl-CoA in 50 mM citrate buffer, pH 5.5 in a final volume of 200 μL. Either 1% ethanol, 250 μM OA or 1 mM DA was added to the reaction, and then the reactions were initiated by adding 0.65 mg/mL OLS. 50 μL aliquots were quenched at 2, 4, 6 and 8 minutes in 150 μL of methanol. The reactions were vortexed briefly and centrifuged at 16,000×g for 2 minutes to pellet the proteins. The supernatant was analyzed by HPLC. The raw peak areas of HTAL, PDAL and olivetol were summed and plotted against time to determine the rate. The rate of the OA supplemented reaction and the DA supplemented reaction were normalized to the ethanol control.

CBGVA Quantification. An authentic CBGVA standard was not immediately available, so a CBGVA standard was generated and quantified using NMR. A 1 mL reaction was set up with AcP, isoprenol and divarinic acid as inputs as described under the isoprenoid reaction conditions above. The reaction was extracted with 3 mL of hexane the hexane dried under argon. The sample was re-dissolved in 500 μL of deuterated methanol with 1 mM 1,3,5-trimethoxybenzene (TMB) as an internal standard. The sample was analyzed using a Bruker AV400 spectrometer. The NMR spectrum matched previously published results, and the CBGVA was quantified by comparison of the singlet hydrogen peak at 6.27 ppm to the internal standard. The quantified CBGVA sample was then used to make an external standard curve on the HPLC.

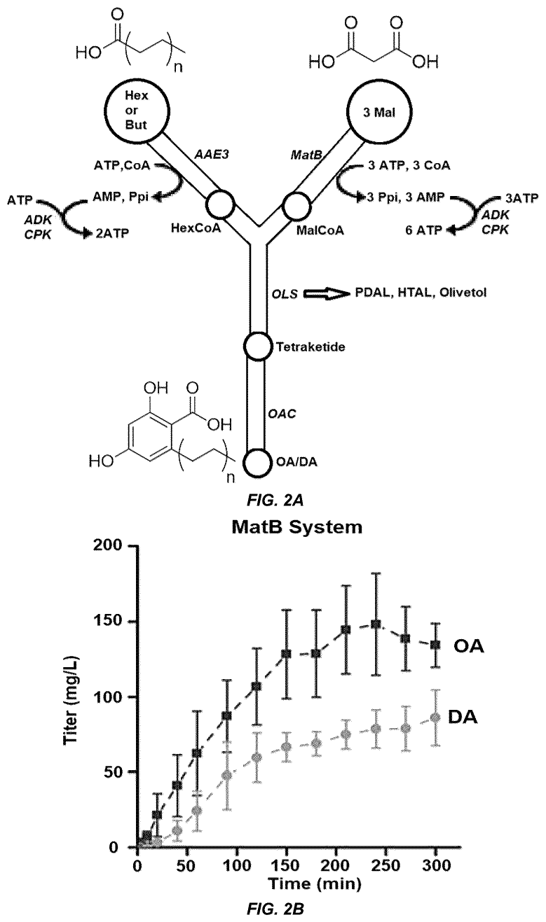

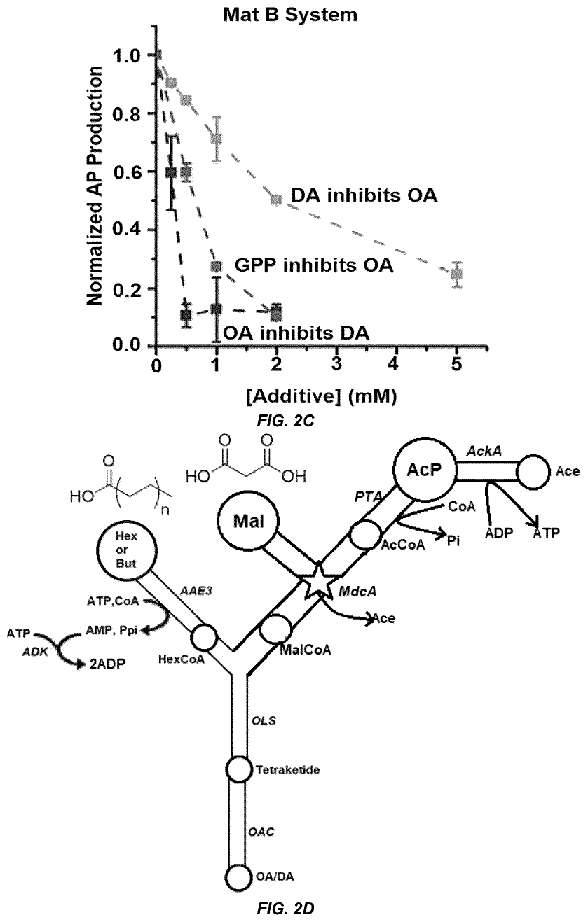

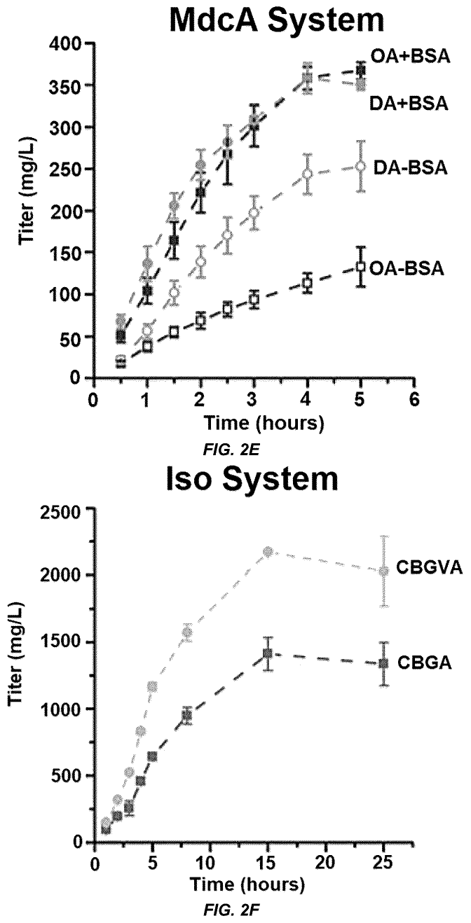

To test and troubleshoot the ability to synthesize OA/DA in vitro, the truncated system shown in FIG. 2A (MatB System) was set up in which malonyl-CoA is generated in the traditional way using MatB, and hexanoyl-CoA (or butyryl-CoA) produced using the acyl activating enzyme AAE3. Hexanyl-CoA (or butyryl-CoA) and malonyl-CoA are employed by olivetolate synthase (OLS) to build a linear tetraketide, which is then converted into OA/DA by olivetolate cyclase (OAC). For this truncated test system ATP was regenerated from AMP using a combination of adenylate kinase (ADK; SEQ ID NO:14 or sequences having 85% to 100% identity thereto) and creatine kinase (CPK) along with the sacrificial substrate creatine phosphate.

Initial reaction conditions were chosen from enzyme specific activities, providing enough inputs to produce up to 5 mM OA. Since MatB and AAE3 compete for ATP and CoA, approximate ratios were targeted that would yield 3 malonyl-CoA per hexanoyl-CoA. The pathway was optimized by individually titrating each reaction component while keeping the remaining components constant. OLS is an imprecise enzyme that releases dead-end side products in addition to the desired tetraketide, and one of the key findings from the optimization process was the importance of balancing the OLS and AAE3 concentration for suppressing side product formation. Experiments showed that as the OLS and AAE3 concentrations increase, the system yields a higher fraction of side products relative to OA (FIG. 4), suggesting that it is critical to tune polyketide initiation, extension and termination events relative to all the other reaction components. FIG. 2B shows the reaction time course for the optimized MatB System. OA production reached a final titer of 148±34 mg/L (660±150 μM) at 2.5 hours, and DA production reached a final titer of 78±12 mg/L (400±61 μM) in 4 hours.

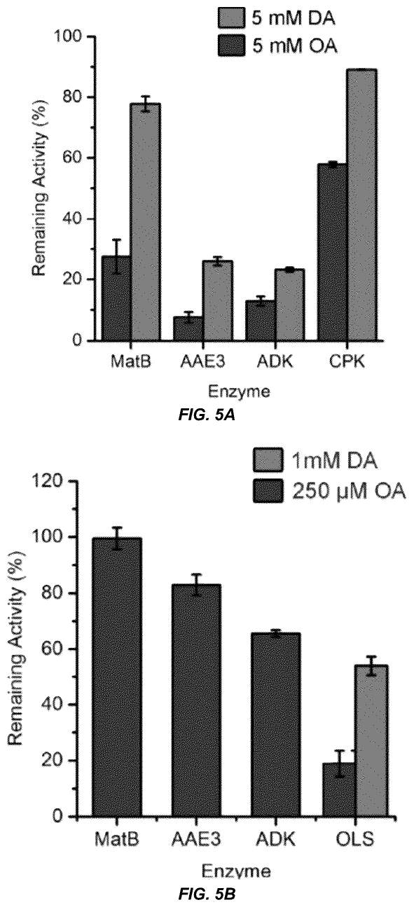

Metabolites were screened for possible inhibition and found that both OA and DA accumulation inhibit the pathway. As shown in FIG. 2C, 1 mM OA reduces DA production by 90%, while DA is a less potent inhibitor, with 1 mM DA reducing OA production 30%. To identify the inhibited enzyme, the individual enzymes were screened in the pathway and found that OA and DA strongly inhibited OLS activity (FIG. 5).

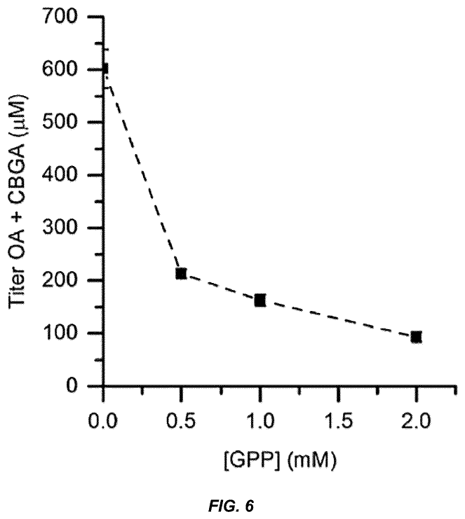

In an effort to reduce OA/DA inhibition, experiments were performed to remove OA/DA from the reaction as it is made by converting it directly into CBGA/CBGVA. To test this GPP and a stabilized CBGA synthase were added to the system (FIG. 6). The CBGA synthase used, NphB M31^{S}, is a stabilized version of the soluble enzyme designed previously (Valliere et al., Nat. Commun. 10:565, 2019). Instead of improved titers, adding more GPP actually yielded less CBGA, indicating that GPP could also inhibit a component of the reaction. Experiments were performed to test the effect of GPP concentration on OA production. At just 500 μM GPP, OA production decreased 40% percent (FIG. 2C). Taken together, the results indicate that high level cannabinoid production in the full pathway will require maintaining low concentrations of OA/DA and GPP during the course of the reaction.

The AP module was then tested with the AR module, including MdcA to reduce ATP consumption (Mdc A System, FIG. 2D). As shown in FIG. 2E, the full AP module yielded 132±24 mg/L of OA or 250±30 mg/L DA in 5 hours, similar to what was observed using MatB for malonyl-CoA production. Additives were screened that might boost performance, focusing on known activators of chalcone synthases (homologous to OLS) since the results suggest that OLS is the most problematic enzyme. The addition of bovine serum albumin (BSA) improved both OA and DA production to 350±10 mg/L. (FIG. 2E).

The ISO and CAN modules were then tested separately from the AP module by supplying OA/DA to the combined ISO/CAN modules externally. The combined ISO module and CAN module system yielded 1350±160 mg/L of CBGA or 2200±261 mg/L of CBGVA in 15 hours (FIG. 2F). These results suggest that the ISO and CAN modules can function efficiently so that the full system performance will likely be limited by the function of the AP module.

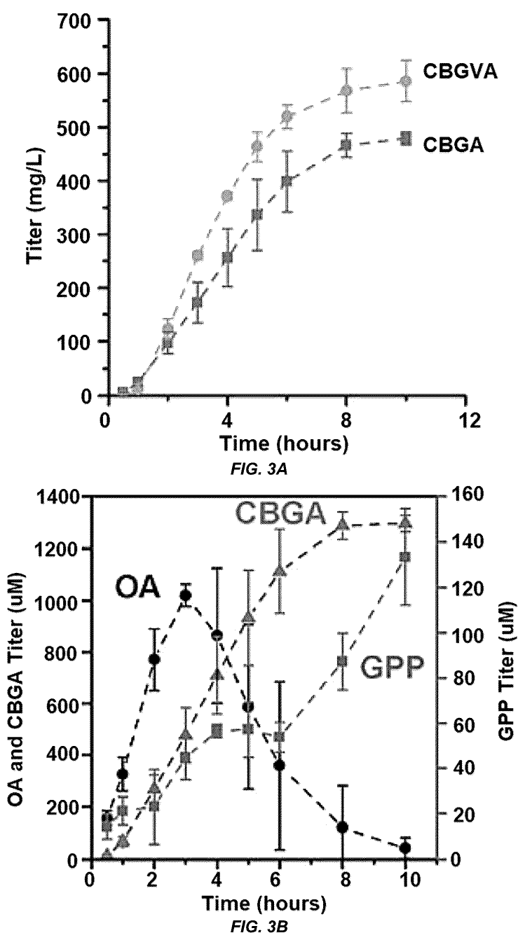

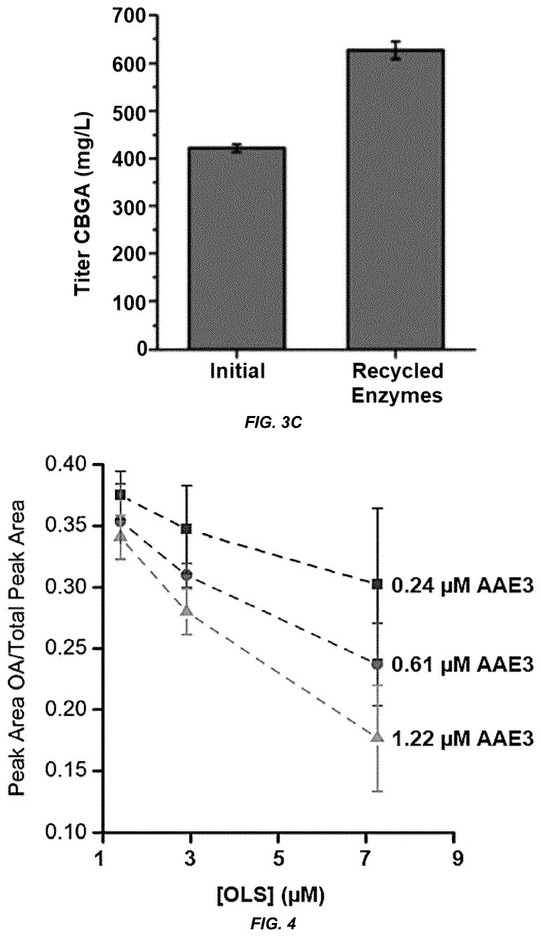

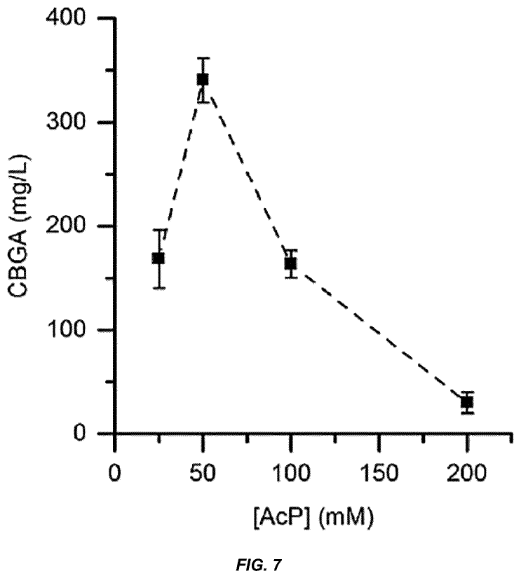

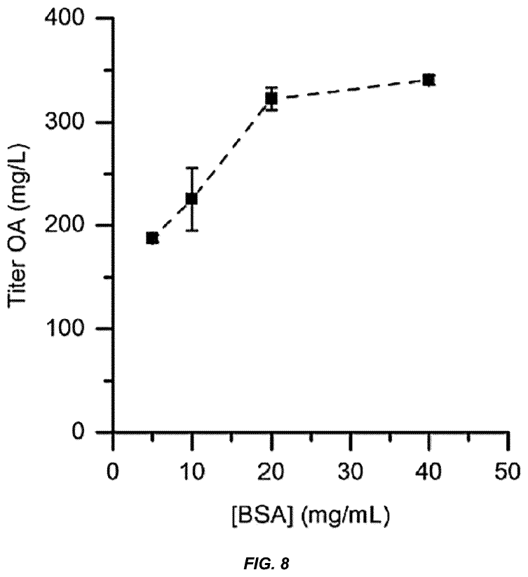

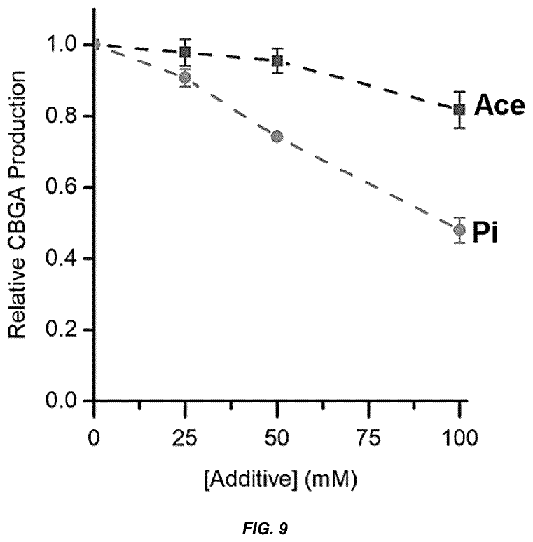

The complete pathway as shown in FIG. 1 was then assembled. After several rounds of optimization, the system generated 480±12 mg/L of CBGA or 580±38 mg/L of CBGVA in 10 hours (FIG. 3A). The starting concentration of AcP was a key factor in optimization as it could not be increased higher than 50 mM without reducing titers (FIG. 7). Additionally, BSA was titered to identify the ideal concentration of 20 mg/mL (FIG. 8). FIG. 3B shows key intermediates, GPP and OA during the time course of CBGA production. OA concentrations spiked early, and then OA decreased with subsequent CBGA production. Once all of the OA was consumed an increase in GPP levels was observed. These results suggest that the ISO module remains functional but the reaction ceases because the AP module becomes dysfunctional. As shown in FIG. 9, phosphate and acetate build up would have minimal effects on the reaction at the concentrations used. To test whether the dysfunction was due to a build-up of other metabolites, the metabolites were removed from a CBGA production system after 6 hours by filtration and restarted the reaction with fresh inputs and cofactors. The recycled enzymes did continue production to a total of 630±20 mg/L of CBGA suggesting that the enzymes remain active (FIG. 3C).

It is encouraging that the cell free system of the disclosure provides cannabinoid titers that are nearly two orders of magnitude higher than those reported in yeast so far and there remains room for further optimization. Moreover, an advantage of the cell free approach is that the problems are well defined. In particular, it is clear that the OLS enzyme is a weak link in the system. The natural enzyme is not only error-prone, readily producing unwanted side products, it is inhibited by key intermediates in the system. It is possible that further tuning of the process could improve results further since the balance of OA/DA and GPP production is an important consideration in OLS function. Alternatively, OLS should be a target of improvement by engineering or directed evolution. Similar considerations led to the development of an efficient water soluble CBGA synthase enzyme employed here to replace the natural integral membrane enzyme. The structure of OLS was recently determined, which could improve engineering efforts. Ideally, both microbial and cell free methods will ultimately become cost competitive so that there can be many viable options for producing these medically important molecules.

Certain embodiments of the invention have been described. It will be understood that various modifications may be made without departing from the spirit and scope of the invention. Other embodiments are within the scope of the following claims.

1. A recombinant polypeptide comprising a sequence selected from the group consisting of:
(i) SEQ ID NO:30 and having a Y288X, A232S and a mutation selected from the group consisting of T69P, T98I and G224S, any combination of the foregoing and all of the foregoing mutations, wherein X is A, N, S, V or a non-natural amino acid;
(ii) SEQ ID NO:30 and having a Y288X, A232S and a mutation selected from the group consisting of T69P, T98I, G224S and T126P, any combination of the foregoing and all of the foregoing mutations, wherein X is A, N, S, V or a non-natural amino acid;
(iii) SEQ ID NO:30 and having a Y288X, A232S and a mutation selected from the group consisting of M14I, Y31W, T69P, T77I, T98I, S136A, E222D, G224S, N236T, G297K, any combination of the foregoing and all of the foregoing mutations, wherein X is A, N, S, V or a non-natural amino acid;
(iv) SEQ ID NO:30 having a Y288X, A232S and a mutation selected from the group consisting of M14I, Y31W, T69P, T77I, E80A, D93S, T98I, T126P, M129L, G131Q, S136A, E222D, G224S, N236T, S277T, G297K, any combination of the foregoing and all of the foregoing mutations, wherein X is A, N, S, V or a non-natural amino acid;
(v) SEQ ID NO:30 having a Y288X, A232S and a mutation selected from the group consisting of M14I, L33I, Y31W, T69P, T77I, V78A, E80A, D93S, T98I, E112G, T114V, T126P, M129L, G131Q, S136A, E222D, G224S, K225Q, N236T, S277T, G297K, any combination of the foregoing and all of the foregoing mutations, wherein X is A, N, S, V or a non-natural amino acid;
(vi) any of (i)-(iv) or (v) comprising from 1-20 conservative amino acid substitutions; and
(vii) a sequence that is at least 85%, 90%, 95%, 98% or 99% identical to the sequences of (i)-(iv) or (v);
wherein the polypeptide of any one of (i) to (vii) has NphB activity.

2. A method of producing CBG(V)A from GPP and Olivetolate (OA) or divarinic acid (DA) or CBGXA from GPP and a 2,4-dihydroxy benzoic acid or derivative thereof comprising incubating GPP and OA, DA or other 2,4-dihydroxy benzoic acid derivatives with a recombinant polypeptide of claim 1 under condition to produce CBG(V)A.

3. A recombinant pathway comprising a polypeptide of claim 1 and a plurality of enzymes that convert isoprenol or prenol to Geranylpyrophosphate (GPP).

4. The recombinant pathway of claim 3, further comprising an ATP regeneration module that converts ADP and/or AMP to ATP.

5. The recombinant pathway of claim 3, wherein the ATP regeneration module converts acetyl-phosphate to acetic acid.

6. The recombinant pathway of claim 3, wherein the pathway comprises the following enzymes:
(i) Acetyl-phosphate transferase (PTA);
(ii) malonate decarboxylase alpha subunit (mdcA);
(iii) acyl activating enzyme 3 (AAE3);
(iv) olivetol synthase (OLS);
(v) olivetolic acid cyclase (OAC);
(vi) hydroxyethylthiazole kinase (ThiM);
(vii) isopentenyl kinase (IPK);
(viii) isopentyl diphosphate isomerase (IDI);
(ix) Diphosphomevalonate decarboxylase alpha subunit (MDCa);
(x) Geranyl-PP synthase (GPPS) or Farnesyl-PP synthease mutant S82F (FPPS S82F); and
(xi) a recombinant polypeptide having a sequence selected from:
(1) SEQ ID NO:30 and having a Y288X, A232S and a mutation selected from the group consisting of T69P, T98I and G224S, any combination of the foregoing and all of the foregoing mutations, wherein X is A, N, S, V or a non-natural amino acid;
(2) SEQ ID NO:30 and having a Y288X, A232S and a mutation selected from the group consisting of T69P, T98I, G224S and T126P, any combination of the foregoing and all of the foregoing mutations, wherein X is A, N, S, V or a non-natural amino acid;
(3) SEQ ID NO:30 and having a Y288X, A232S and a mutation selected from the group consisting of M14I, Y31W, T69P, T77I, T98I, S136A, E222D, G224S, N236T, G297K, any combination of the foregoing and all of the foregoing mutations, wherein X is A, N, S, V or a non-natural amino acid;
(4) SEQ ID NO:30 having a Y288X, A232S and a mutation selected from the group consisting of M14I, Y31W, T69P, T77I, E80A, D93S, T98I, T126P, M129L, G131Q, S136A, E222D, G224S, N236T, S277T, G297K, any combination of the foregoing and all of the foregoing mutations, wherein X is A, N, S, V or a non-natural amino acid;
(5) SEQ ID NO:30 having a Y288X, A232S and a mutation selected from the group consisting of M14I, L33I, Y31W, T69P, T77I, V78A, E80A, D93S, T98I, E112G, T114V, T126P, M129L, G131Q, S136A, E222D, G224S, K225Q, N236T, S277T, G297K, any combination of the foregoing and all of the foregoing mutations, wherein X is A, N, S, V or a non-natural amino acid;
(6) any of (1)-(4) or (5) comprising from 1-20 conservative amino acid substitutions;
(7) a sequence that is at least 85%, 90%, 95%, 98% or 99% identical to the sequences of (1)-(4) or (5);
wherein the polypeptide of any one of (1) to (7) has NphB activity.

7. The recombinant pathway of claim 6, wherein the pathway is supplemented with BSA.

8. The recombinant pathway of claim 6, wherein the pathway is supplemented with acetyl-phosphate, malonate, hexanoate or butyrate, and prenol or isoprenol.

9. The recombinant pathway of claim 8, wherein the pathway further comprises a cannabidiolic acid synthase.

10. The recombinant pathway of claim 9, wherein the pathway produces cannabidiolic acid.

11. A cell free enzymatic system for the production of cannabigerolic acid or cannabigerovarinic acid, the pathway including
(i) Acetyl-phosphate transferase (PTA);
(ii) malonate decarboxylase alpha subunit (mdcA);
(iii) acyl activating enzyme 3 (AAE3);
(iv) olivetol synthase (OLS);
(v) olivetolic acid cyclase (OAC);
(vi) hydroxyethylthiazole kinase (ThiM);
(vii) isopentenyl kinase (IPK);
(viii) isopentyl diphosphate isomerase (IDI);
(ix) Diphosphomevalonate decarboxylase alpha subunit (MDCa);
(x) Geranyl-PP synthase (GPPS) or Farnesyl-PP synthease mutant S82F (FPPS S82F); and
(xi) a recombinant polypeptide comprising a sequence selected from the group consisting of:
(1) SEQ ID NO:30 and having a Y288X, A232S and a mutation selected from the group consisting of T69P, T98I and G224S, any combination of the foregoing and all of the foregoing mutations, wherein X is A, N, S, V or a non-natural amino acid;
(2) SEQ ID NO:30 and having a Y288X, A232S and a mutation selected from the group consisting of T69P, T98I, G224S and T126P, any combination of the foregoing and all of the foregoing mutations, wherein X is A, N, S, V or a non-natural amino acid;
(3) SEQ ID NO:30 and having a Y288X, A232S and a mutation selected from the group consisting of M14I, Y31W, T69P, T77I, T98I, S136A, E222D, G224S, N236T, G297K, any combination of the foregoing and all of the foregoing mutations, wherein X is A, N, S, V or a non-natural amino acid;
(4) SEQ ID NO:30 having a Y288X, A232S and a mutation selected from the group consisting of M14I, Y31W, T69P, T77I, E80A, D93S, T98I, T126P, M129L, G131Q, S136A, E222D, G224S, N236T, S277T, G297K, any combination of the foregoing and all of the foregoing mutations, wherein X is A, N, S, V or a non-natural amino acid;
(5) SEQ ID NO:30 having a Y288X, A232S and a mutation selected from the group consisting of M14I, L33I, Y31W, T69P, T77I, V78A, E80A, D93S, T98I, E112G, T114V, T126P, M129L, G131Q, S136A, E222D, G224S, K225Q, N236T, S277T, G297K, any combination of the foregoing and all of the foregoing mutations, wherein X is A, N, S, V or a non-natural amino acid;
(6) any of (1)-(4) or (5) comprising from 1-20 conservative amino acid substitutions;
(7) a sequence that is at least 85%, 90%, 95%, 98% or 99% identical to the sequences of (1)-(4) or (5);
wherein the polypeptide of any one of (1) to (7) has NphB activity.

12. An isolated polynucleotide encoding a polypeptide selected from the group consisting of:
(1) SEQ ID NO:30 and having a Y288X, A232S and a mutation selected from the group consisting of T69P, T98I and G224S, any combination of the foregoing and all of the foregoing mutations, wherein X is A, N, S, V or a non-natural amino acid;
(2) SEQ ID NO:30 and having a Y288X, A232S and a mutation selected from the group consisting of T69P, T98I, G224S and T126P, any combination of the foregoing and all of the foregoing mutations, wherein X is A, N, S, V or a non-natural amino acid;
(3) SEQ ID NO:30 and having a Y288X, A232S and a mutation selected from the group consisting of M14I, Y31W, T69P, T77I, T98I, S136A, E222D, G224S, N236T, G297K, any combination of the foregoing and all of the foregoing mutations, wherein X is A, N, S, V or a non-natural amino acid;
(4) SEQ ID NO:30 having a Y288X, A232S and a mutation selected from the group consisting of M14I, Y31W, T69P, T77I, E80A, D93S, T98I, T126P, M129L, G131Q, S136A, E222D, G224S, N236T, S277T, G297K, any combination of the foregoing and all of the foregoing mutations, wherein X is A, N, S, V or a non-natural amino acid;
(5) SEQ ID NO:30 having a Y288X, A232S and a mutation selected from the group consisting of M14I, L33I, Y31W, T69P, T77I, V78A, E80A, D93S, T98I, E112G, T114V, T126P, M129L, G131Q, S136A, E222D, G224S, K225Q, N236T, S277T, G297K, any combination of the foregoing and all of the foregoing mutations, wherein X is A, N, S, V or a non-natural amino acid;
(6) any of (1)-(4) or (5) comprising from 1-20 conservative amino acid substitutions;
(7) a sequence that is at least 85%, 90%, 95%, 98% or 99% identical to the sequences of (1)-(4) or (5);
wherein the polypeptide of any one of (1) to (7) has NphB activity.

13. A vector comprising the isolated polynucleotide of claim 12.

14. A recombinant microorganism comprising the isolated polynucleotide of claim 12.

15. A recombinant microorganism comprising the vector of claim 13.

16. An artificial in vitro enzymatic pathway for the production of CBG(X)A, the pathway comprising:
(a)(1) an enzyme that converts prenol and ATP to prenol phosphate and ADP, an enzyme that converts prenol phosphate and ATP to dimethylallyl diphosphate (DMAPP), and/or (2) an enzyme that converts isoprenol and ATP to isoprenol phosphate and ADP and an enzyme that converts isoprenol phosphate and ATP to isopentenyl diphosphate (IPP);
(b) an enzyme that isomerizes DMAPP to IPP and/or IPP to DMAPP when only prenol or isoprenol are present;
(c) an enzyme that converts DMAPP and IPP to geranyl pyrophosphate (GPP); and
(d) an enzyme that converts GPP and olivetolic acid or divarinic acid or similar compound to CBG(X)A or variant thereof.

17. The artificial in vitro enzymatic pathway of claim 16, wherein the input substrate(s) are olivetolic acid, divarinic acid, 2,4 dihydroxybenzoic acid derivative, prenol and/or isoprenol.

18. The artificial in vitro enzymatic pathway of claim 17, further comprising at ATP generating system that converts that ADP from part (a) to ATP.

19. The artificial in vitro enzymatic pathway of claim 16, wherein the enzyme that converts GPP and olivetolic acid or divarinic acid or other 2,4 dihydroxybenzoic acid derivative comprises a recombinant polypeptide having a sequence selected from the group consisting of:
(1) SEQ ID NO:30 and having a Y288X, A232S and a mutation selected from the group consisting of T69P, T98I and G224S, any combination of the foregoing and all of the foregoing mutations, wherein X is A, N, S, V or a non-natural amino acid;
(2) SEQ ID NO:30 and having a Y288X, A232S and a mutation selected from the group consisting of T69P, T98I, G224S and T126P, any combination of the foregoing and all of the foregoing mutations, wherein X is A, N, S, V or a non-natural amino acid;
(3) SEQ ID NO:30 and having a Y288X, A232S and a mutation selected from the group consisting of M14I, Y31W, T69P, T77I, T98I, S136A, E222D, G224S, N236T, G297K, any combination of the foregoing and all of the foregoing mutations, wherein X is A, N, S, V or a non-natural amino acid;
(4) SEQ ID NO:30 having a Y288X, A232S and a mutation selected from the group consisting of M14I, Y31W, T69P, T77I, E80A, D93S, T98I, T126P, M129L, G131Q, S136A, E222D, G224S, N236T, S277T, G297K, any combination of the foregoing and all of the foregoing mutations, wherein X is A, N, S, V or a non-natural amino acid;
(5) SEQ ID NO:30 having a Y288X, A232S and a mutation selected from the group consisting of M14I, L33I, Y31W, T69P, T77I, V78A, E80A, D93S, T98I, E112G, T114V, T126P, M129L, G131Q, S136A, E222D, G224S, K225Q, N236T, S277T, G297K, any combination of the foregoing and all of the foregoing mutations, wherein X is A, N, S, V or a non-natural amino acid;
(6) any of (1)-(4) or (5) comprising from 1-20 conservative amino acid substitutions;
(7) a sequence that is at least 85%, 90%, 95%, 98% or 99% identical to the sequences of (1)-(4) or (5);
wherein the polypeptide of any one of (1) to (7) has NphB activity.

20. A enzymatic pathway as set forth in FIG. 1A-B.


SEQUENCE LISTING (75 sequences)

SEQ ID NO: 1 - DNA, 789 bp, Escherichia coli
  CDS (1)..(789)
atgcaagtcgacctgctgggttcagcgcaatctgcgcacgcgttacacctttttcaccaacattcccctcttgtgcactgcatgaccaatgatgtggtgcaaacctttaccgccaataccttgctggcgctcggtgcatcgccagcgatggttatcgaaaccgaagaggccagtcagtttgcggctatcgccagtgccttgttgattaacgttggcacactgacgcagccacgcgctcaggcgatgcgtgctgccgttgagcaagcaaaaagctctcaaacaccctggacgcttgatccagtagcggtgggtgcgctcgattatcgccgccatttttgtcatgaacttttatcttttaaaccggcagcgatacgtggtaatgcttcggaaatcatggcattagctggcattgctaatggcggacggggagtggataccactgacgccgcagctaacgcgatacccgctgcacaaacactggcacgggaaactggcgcaatcgtcgtggtcactggcgagatggattatgttaccgatggacatcgtatcattggtattcacggtggtgatccgttaatgaccaaagtggtaggaactggctgtgcattatcggcggttgtcgctgcctgctgtgcgttaccaggcgatacgctggaaaatgtcgcatctgcctgtcactggatgaaacaagccggagaacgcgcagtcgccagaagcgaggggccaggcagttttgttccacatttccttgatgcgctctggcaattgacgcaggaggtgcaggcataa

SEQ ID NO: 2 - protein, 262 aa, Escherichia coli
MQVDLLGSAQSAHALHLFHQHSPLVHCMTNDVVQTFTANTLLALGASPAMVIETEEASQFAAIASALLINVGTLTQPRAQAMRAAVEQAKSSQTPWTLDPVAVGALDYRRHFCHELLSFKPAAIRGNASEIMALAGIANGGRGVDTTDAAANAIPAAQTLARETGAIVVVTGEMDYVTDGHRIIGIHGGDPLMTKVVGTGCALSAVVAACCALPGDTLENVASACHWMKQAGERAVARSEGPGSFVPHFLDALWQLTQEVQA

SEQ ID NO: 3 - DNA, 1692 bp, Cannabis sativa
  CDS (1)..(1692)
atgggcagcagccatcatcatcatcatcacagcagcggcctggtgccgcgcggcagccatatggaaaagagtggctacggacgcgacggtatttaccgtagcctgcgtcctcctttacacctgccaaacaataacaatttgagtatggtctcattcctgttccgtaacagcagcagctatccacagaaaccggcgttgatcgatagcgagactaatcaaattttatcttttagtcattttaaaagcaccgtgatcaaggtctcccatggcttcttaaacctggggatcaaaaagaatgacgtggttttaatctacgcacccaattcgatccactttcccgtatgcttccttggcattattgcttctggggcgatcgccactacttcaaatccattatacaccgtgagtgagttgtcgaaacaagtaaaggactcgaaccctaaattgattatcacagtccctcagttattggaaaaggtcaagggtttcaatctgccaactatccttatcggccctgattctgagcaggaatcgtctagtgataaagtaatgactttcaatgatctggtcaatctgggaggaagttcgggtagcgaattccctatcgtcgacgatttcaagcaatccgacaccgccgcactgttgtactcaagtggcacgacaggtatgagcaagggggtcgttctgacgcacaaaaattttattgcctcatcgttgatggtaacaatggaacaggacttggtcggcgagatggacaatgtgttcctgtgtttccttcctatgtttcacgtctttggcttagccattattacgtatgctcagttacagcgcggtaataccgtgatttcaatggcccgctttgacttggaaaagatgttaaaagatgttgaaaagtacaaagttacccacctttgggtcgtacccccagttatcttagcgttgtcgaagaactcaatggtgaaaaaattcaatttgtcatccatcaagtatattggttcaggcgctgcgccattaggaaaggatctgatggaagaatgctctaaggtggttccttacggaatcgtggctcaaggatatggcatgacggaaacgtgcggaatcgtatccatggaagacatccgcggcgggaaacgcaattcagggtcggccggaatgttggcaagtggggtagaagctcagatcgtgagtgtggacaccttaaaaccccttcccccgaatcaattaggggaaatctgggtaaaaggtccaaatatgatgcaaggctatttcaacaatcctcaagcgaccaaacttaccattgataaaaagggttgggttcatactggcgacttggggtatttcgacgaagacggacacttatatgttgtagaccgtattaaggagcttattaaatacaagggattccaagttgcgcctgcggaactggagggattattagttagtcaccccgagatcttagacgcggtagttattcccttccccgatgctgaggcaggcgaagtcccggtggcatacgttgttcgctcgcctaacagttcgttgaccgaaaatgacgttaaaaaattcatcgccggtcaggtcgcctcctttaagcgtctgcgcaaggttacttttattaattccgtccccaagagcgcaagtgggaagattctgcgccgcgagcttattcaaaaggttcgctctaacatgtaa

SEQ ID NO: 4 - protein, 563 aa, Cannabis sativa
MGSSHHHHHHSSGLVPRGSHMEKSGYGRDGIYRSLRPPLHLPNNNNLSMVSFLFRNSSSYPQKPALIDSETNQILSFSHFKSTVIKVSHGFLNLGIKKNDVVLIYAPNSIHFPVCFLGIIASGAIATTSNPLYTVSELSKQVKDSNPKLIITVPQLLEKVKGFNLPTILIGPDSEQESSSDKVMTFNDLVNLGGSSGSEFPIVDDFKQSDTAALLYSSGTTGMSKGVVLTHKNFIASSLMVTMEQDLVGEMDNVFLCFLPMFHVFGLAIITYAQLQRGNTVISMARFDLEKMLKDVEKYKVTHLWVVPPVILALSKNSMVKKFNLSSIKYIGSGAAPLGKDLMEECSKVVPYGIVAQGYGMTETCGIVSMEDIRGGKRNSGSAGMLASGVEAQIVSVDTLKPLPPNQLGEIWVKGPNMMQGYFNNPQATKLTIDKKGWVHTGDLGYFDEDGHLYVVDRIKELIKYKGFQVAPAELEGLLVSHPEILDAVVIPFPDAEAGEVPVAYVVRSPNSSLTENDVKKFIAGQVASFKRLRKVTFINSVPKSASGKILRRELIQKVRSNM

SEQ ID NO: 5 - DNA, 1713 bp, Geobacillus sp. 44B
  CDS (1)..(1713)
atgggcagcagccatcatcatcatcatcacagcagcggcctggtgccgcgcggcagccatatgaatagaatacaccggtctaaacgttcatggacaacgcgtcgcgatgcgaaggcaaagcgaatggcaaaattggagcgagtcgtgaacggaaaaattataccaacagataaaattgtagaggcattagaagcggttattgctccaggggatcgtgttgtgttagaaggaaataatcaaaaacaagcttcgtttctatccaaggcattatccaaagttaaccctgagaaagtgaacggattacatatgattatgtccagtgtatcgcgaccagagcatttagatatatttgaaaaaggaatcgctagaaaaattgatttttcttatgccggcccacaaagtcttcgcatgtcacaaatgctggaagacggaaagcttattataggggaaatccatacctatcttgagctatatgggcggttatttattgatttgactccgtctgttgcactagtggcggcggataaagcagaccgatcgggcaatttgtatacaggacctaatacagaggaaactccaacgcttgttgaagctacggcattccgggacggaatcgttatagcccaagtaaatgaactggcagatgaactgccacgggtagatatacctggctcttggattgattttatcgttgttgctgaccagccttatgaattagaacctctttttacaagagatcctcgccttattacagaaatccagattcttatggcgatgatgacgattagagggatatatgaacgtcataacatccaatctctcaaccatggaatcggatttaatactgcggcgattgagttattgcttccaacgtacggagaatcattaggattgaaggggaaaatttgcagacattgggcattgaatccgcatcctacccttataccagctattgaaacaggatgggtagaaagcattcattgttttggaggagaagtaggaatggaaaagtatattgcggcacgtcccgatgtgttctttactggaaaagatgggagtttacgttcaaaccgggcattatcccaagtagctggacagtatgctgtcgatctttttatcggttctactctacagatggatagggatgggaattcttcaacagtaacgattggaagactggcaggattcggcggggcaccaaacatggggcatgatcctcgtggacggcgccattccactcctgcatggctagatatgataacgtccgatcatccgatcgcgaaaggaaaaaaattagtcgtgcagatagtagaaacgtttcaaaaaggaaatcgaccggtatttgttgagtctttagatgcgattgaagtagggaaaaaggcgaatttggcgacagcgccaattatgatatatggggatgatgtgacccatgttgtcactgaagaaggaatcgcatatttgtataaggcgaatagtttagaagaacgccgtcaggccattgcggcaatcgccggagtcacaccgattgggctagaacatgatccaaaaagaactgagcagttgcgaagggatggattggtggcgtttccggaggatttaggcatacgccgtaccgatgccaaacgttctttattagcagcaaaaagcattgaagaactggttgaatggtcggagggattgtatgaaccgccggctagatttcgcagctggtaa

SEQ ID NO: 6 - protein, 570 aa, Geobacillus sp. 44B
MGSSHHHHHHSSGLVPRGSHMNRIHRSKRSWTTRRDAKAKRMAKLERVVNGKIIPTDKIVEALEAVIAPGDRVVLEGNNQKQASFLSKALSKVNPEKVNGLHMIMSSVSRPEHLDIFEKGIARKIDFSYAGPQSLRMSQMLEDGKLIIGEIHTYLELYGRLFIDLTPSVALVAADKADRSGNLYTGPNTEETPTLVEATAFRDGIVIAQVNELADELPRVDIPGSWIDFIVVADQPYELEPLFTRDPRLITEIQILMAMMTIRGIYERHNIQSLNHGIGFNTAAIELLLPTYGESLGLKGKICRHWALNPHPTLIPAIETGWVESIHCFGGEVGMEKYIAARPDVFFTGKDGSLRSNRALSQVAGQYAVDLFIGSTLQMDRDGNSSTVTIGRLAGFGGAPNMGHDPRGRRHSTPAWLDMITSDHPIAKGKKLVVQIVETFQKGNRPVFVESLDAIEVGKKANLATAPIMIYGDDVTHVVTEEGIAYLYKANSLEERRQAIAAIAGVTPIGLEHDPKRTEQLRRDGLVAFPEDLGIRRTDAKRSLLAAKSIEELVEWSEGLYEPPARFRSW

SEQ ID NO: 7 - DNA, 1038 bp, Geobacillus stearothermophilus
  CDS (1)..(1038)
atgggcagcagccatcatcatcatcatcacagcagcggcctggtgccgcgcggcagccatatgacaaccgatttatttacggcattaaaagcgaaagtaaccggtacggctcgaaaaatcgtgtttcccgagggaaccgatgaccgcatcttaacggcggcgagccgtttggcgacggagcaagtgcttcagccgatcgtccttggcgatgagcaagcgataagggtgaaagcagctgcgcttggcttgccgcttgaaggggtggagattgtcaacccgcgccgctacggcgggtttgatgagctagtttcggcgtttgtggagcggcgcaaagggaaagtgacagaagaaacggcgcgcgagttgcttttcgatgaaaactatttcggtacgatgctcgtttatatgggagcggccgacggcctcgtcagcggggcggcacattcgacggcggatacggtccgaccagccttgcaaatcattaaaacgaagccaggcgttgacaaaacgtccggcgtgttcatcatggtgcgcggcgacgaaaaatatgtgtttgccgattgcgccatcaacattgctcctaacagtcatgatttggctgaaatcgcggtcgagagcgcccggacggccaaaatgttcggccttaagccgcgcgtagtgctgttaagcttttccacgaaagggtcggcctcgtcgccggagacggaaaaagtcgttgaggcggtgcggttggcgaaagaaatggcgccggatctgatccttgacggtgagtttcaatttgacgccgcgtttgtgccagaggtggcgaaaaagaaagcgccggactcggtcattcaaggggacgcaaatgtctttattttcccgagccttgaggcgggcaacatcggctacaaaatcgcccagcgccttggcggctttgaagcggttggcccgattttgcaagggctgaacaagccggttaacgacctatcgcgcggctgcagcgccgaagacgcctacaagctcgcgctcatcaccgcggcgcagtcgcttggggag

SEQ ID NO: 8 - protein, 346 aa, Geobacillus stearothermophilus
MGSSHHHHHHSSGLVPRGSHMTTDLFTALKAKVTGTARKIVFPEGTDDRILTAASRLATEQVLQPIVLGDEQAIRVKAAALGLPLEGVEIVNPRRYGGFDELVSAFVERRKGKVTEETARELLFDENYFGTMLVYMGAADGLVSGAAHSTADTVRPALQIIKTKPGVDKTSGVFIMVRGDEKYVFADCAINIAPNSHDLAEIAVESARTAKMFGLKPRVVLLSFSTKGSASSPETEKVVEAVRLAKEMAPDLILDGEFQFDAAFVPEVAKKKAPDSVIQGDANVFIFPSLEAGNIGYKIAQRLGGFEAVGPILQGLNKPVNDLSRGCSAEDAYKLALITAAQSLGE

SEQ ID NO: 9 - DNA, 1218 bp, Cannibis sativa
  CDS (1)..(1218)
atgggcagcagccatcatcatcatcatcacagcagcggcctggtgccgcgcggcagccatatgaatcatctgcgtgctgaaggaccagcttccgtattggcaattggaacagctaaccctgagaacattcttcttcaggatgagtttcccgactattacttccgcgtgacaaagagcgaacacatgacacagcttaaagagaagttccgtaagatctgtgacaaaagcatgatccgcaaacgtaactgcttccttaacgaggagcatctgaagcagaatccccgtcttgttgaacatgagatgcagaccttggatgctcgccaggacatgttggttgttgaggtccctaagctgggcaaagatgcgtgtgcaaaagcgattaaagagtgggggcagcctaaaagcaaaattactcatctgattttcacaagcgccagtacaaccgatatgcccggtgcggactaccattgtgcaaaattattgggtttatcgccttcagtaaaacgtgttatgatgtaccagttaggatgctacggtggtggcaccgtacttcgtattgcgaaggacatcgccgagaacaacaaaggagcccgtgtacttgctgtatgttgtgatatcatggcgtgcctttttcgcggccccagcgagagtgaccttgagttacttgtggggcaggccatcttcggagacggtgccgcagccgtcattgttggcgcagagcccgatgaatccgttggcgagcgcccgatctttgagcttgtaagtacaggacaaactatcttgcccaactctgaggggactatcggcggacatattcgtgaggcgggcttgatttttgaccttcacaaggatgttccaatgcttatctccaataatattgaaaaatgtcttatcgaagcattcactccgattggtatctccgattggaattcgattttttggatcacccatcctggtgggaaagctattttagacaaggtggaggagaaattacatcttaagtcagataagtttgtcgacagtcgccacgtgttgtcggaacatggcaacatgtcatcgtcaaccgtcttgttcgttatggacgaattacgtaaacgcagtttagaagagggtaagagtacgacgggggacgggttcgagtggggagtcttattcgggttcggtccaggattgacagtggaacgcgtcgtggttcgcagtgtccccattaagtactaa

SEQ ID NO: 10 - protein, 405 aa, Cannibis sativa
MGSSHHHHHHSSGLVPRGSHMNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 11 - DNA, 366 bp, Cannibis sativa
  CDS (1)..(366)
atgggcagcagccatcatcatcatcatcacagcagcggcctggtgccgcgcggcagccatatggcagtcaaacacttgatcgtgttaaagttcaaagatgaaatcacagaggctcagaaggaagaatttttcaagacgtatgtaaaccttgttaatatcatccccgctatgaaggatgtgtattggggtaaagacgtgacacagaagaacaaagaggaaggctacacgcacatcgtagaggtcacatttgagagcgtcgaaactattcaggattacatcattcatcccgcacacgttggattcggggatgtgtatcgctctttctgggaaaaattgctgatcttcgactatacaccgcgtaagtaa

SEQ ID NO: 12 - protein, 121 aa, Cannibis sativa
MGSSHHHHHHSSGLVPRGSHMAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 13 - DNA, 654 bp, Geobacillus thermodenitrificans
  CDS (1)..(654)
atgaatttagtgctgatggggctgccaggtgccggcaaaggcacgcaagccgagaaaatcgtagaaacgtatggaatcccacatatttcaaccggggatatgtttcgggcggcgatgaaagaaggcacaccgttaggattgcaggcaaaagaatatatcgaccgtggtgatcttgttccggatgaggtgacgatcggtatcgtccgtgaacggttaagcaaagacgactgccaaaacggctttttgcttgacggattcccacgcacggttgcccaagcggaggcgctggaagcgatgctggctgaaatcggccgcaagcttgactatgtcatccatatcgatgttcgccaagatgtgttaatggagcgcctcacaggcagacgaatttgtcgcaactgcggagcgacataccatcttgtttttcacccaccggctcagccaggcgtatgtgataaatgcggtggcgagctttatcagcgccctgacgataatgaagcaacagtggcgaatcggcttgaggtgaatacgaaacaaatgaagccattgctcgatttctatgagcaaaaaggctatttgcgccacattaacggcgaacaagaaatggaaaaagtgtttagcgacattcgcgaattgctcgggggacttactcgatga

SEQ ID NO: 14 - protein, 217 aa, Geobacillus thermodenitrificans
MNLVLMGLPGAGKGTQAEKIVETYGIPHISTGDMFRAAMKEGTPLGLQAKEYIDRGDLVPDEVTIGIVRERLSKDDCQNGFLLDGFPRTVAQAEALEAMLAEIGRKLDYVIHIDVRQDVLMERLTGRRICRNCGATYHLVFHPPAQPGVCDKCGGELYQRPDDNEATVANRLEVNTKQMKPLLDFYEQKGYLRHINGEQEMEKVFSDIRELLGGLTR

SEQ ID NO: 15 - DNA, 1512 bp, Rhodopseudomonas palustris
  CDS (1)..(1512)
atgaacgccaacctgttcgcccgcctgttcgataagctcgacgacccccacaagctcgcgatcgaaaccgcggccggggacaagatcagctacgccgagctggtggcgcgggcgggccgcgtcgccaacgtgctggtggcacgcggcctgcaggtcggcgaccgcgttgcggcgcaaaccgagaagtcggtggaagcgctggtgctgtatctcgccacggtgcgggccggcggcgtgtatctgccgctcaacaccgcctatacgctgcacgagctcgattacttcatcaccgatgccgagccgaagatcgtggtgtgcgatccgtccaagcgcgacgggatcgcggcgattgccgccaaggtcggcgccacggtggagacgcttggccccgacggtcggggctcgctcaccgatgcggcagctggagccagcgaggcgttcgccacgatcgaccgcggcgccgatgatctggcggcgatcctctacacctcagggacgaccggccgctccaagggcgcgatgctcagccacgacaatttggcgtcgaactcgctgacgctggtcgattactggcgcttcacgccggatgacgtgctgatccacgcgctgccgatctatcacacccatggattgttcgtggccagcaacgtcacgctgttcgcgcgcggatcgatgatcttcctgccgaagttcgatcccgacaagatcctcgacctgatggcgcgcgccaccgtgctgatgggtgtgccgacgttctacacgcggctcttgcagagcccgcggctgaccaaggagacgacgggccacatgaggctgttcatctccgggtcggcgccgctgctcgccgatacgcatcgcgaatggtcggcgaagaccggtcacgccgtgctcgagcgctacggcatgaccgagaccaacatgaacacctcgaacccgtatgacggcgaccgcgtccccggcgcggtcggcccggcgctgcccggcgtttcggcgcgcgtgaccgatccggaaaccggcaaggaactgccgcgcggcgacatcgggatgatcgaggtgaagggcccgaacgtgttcaagggctactggcggatgccggagaagaccaagtctgaattccgcgacgacggcttcttcatcaccggcgacctcggcaagatcgacgagcgcggctacgtccacatcctcggccgcggcaaggatctggtgatcaccggcggcttcaacgtctatccgaaggaaatcgagagcgagatcgacgccatgccgggcgtggtcgaatccgcggtgatcggcgtgccgcacgccgatttcggcgagggcgtcactgccgtggtggtgcgcgacaagggtgccacgatcgacgaagcgcaggtgctgcacggcctcgacggtcagctcgccaagttcaagatgccgaagaaagtgatcttcgtcgacgacctgccgcgcaacaccatgggcaaggtccagaagaacgtcctgcgcgagacctacaaggacatctacaagtaa

SEQ ID NO: 16 - protein, 503 aa, Rhodopseudomonas palustris
MNANLFARLFDKLDDPHKLAIETAAGDKISYAELVARAGRVANVLVARGLQVGDRVAAQTEKSVEALVLYLATVRAGGVYLPLNTAYTLHELDYFITDAEPKIVVCDPSKRDGIAAIAAKVGATVETLGPDGRGSLTDAAAGASEAFATIDRGADDLAAILYTSGTTGRSKGAMLSHDNLASNSLTLVDYWRFTPDDVLIHALPIYHTHGLFVASNVTLFARGSMIFLPKFDPDKILDLMARATVLMGVPTFYTRLLQSPRLTKETTGHMRLFISGSAPLLADTHREWSAKTGHAVLERYGMTETNMNTSNPYDGDRVPGAVGPALPGVSARVTDPETGKELPRGDIGMIEVKGPNVFKGYWRMPEKTKSEFRDDGFFITGDLGKIDERGYVHILGRGKDLVITGGFNVYPKEIESEIDAMPGVVESAVIGVPHADFGEGVTAVVVRDKGATIDEAQVLHGLDGQLAKFKMPKKVIFVDDLPRNTMGKVQKNVLRETYKDIYK

SEQ ID NO: 17 - DNA, 558 bp, Geobacillus stearothermophilus
  CDS (1)..(558)
atgggcagcagccatcatcatcatcatcacagcagcggcctggtgccgcgcggcagccatatggcctttgagaataagattgtcgaagcgtttatcgaaattccaaccggcagccaaaacaaatacgagttcgacaaagagcggggcgttttcaaactcgaccgcgtcttgtactccccgatgttttacccggctgagtacggctacttgcaaaatacgctggcgctcgatggcgacccgctcgacattttggtcatcacaacgaatccgacattcccgggctgcgtcatcgatacgcgtgtcatcggctttttgaacatggtcgacagcggtgaggaggacgcgaagctcatcggcgtgccagtcgaagacccgcgctttgatgaagtccgctcgattgaagacctgccgcagcacaagctgaaagaaatcgcccacttctttgaacggtacaaagacttgcaaggcaagcggacggaaatcggcacatgggaagggccggaagctgcggcaaaactgatcgatgagtgcatcgcccgctataacgaacaaaaataa

SEQ ID NO: 18 - protein, 185 aa, Geobacillus stearothermophilus
MGSSHHHHHHSSGLVPRGSHMAFENKIVEAFIEIPTGSQNKYEFDKERGVFKLDRVLYSPMFYPAEYGYLQNTLALDGDPLDILVITTNPTFPGCVIDTRVIGFLNMVDSGEEDAKLIGVPVEDPRFDEVRSIEDLPQHKLKEIAHFFERYKDLQGKRTEIGTWEGPEAAAKLIDECIARYNEQK

SEQ ID NO: 19 - DNA, 954 bp, Artificial Sequence
  GsFPPS-S82F Coding Sequence
atgggcagcagccatcatcatcatcatcacagcagcggcctggtgccgcgcggcagccatatggcgcagctttcagttgaacagtttctcaacgagcaaaaacaggcggtggaaacagcgctctcccgttatatagagcgcttagaagggccggcgaagctgaaaaaggcgatggcgtactcattggaggccggcggcaaacgaatccgtccgttgctgcttctgtccaccgttcgggcgctcggcaaagacccggcggtcggattgcccgtcgcctgcgcgattgaaatgatccatacgtactttttgatccatgatgatttgccgagcatggacaacgatgatttgcggcgcggcaagccgacgaaccataaagtgttcggcgaggcgatggccatcttggcgggggacgggttgttgacgtacgcgtttcaattgatcaccgaaatcgacgatgagcgcatccctccttccgtccggcttcggctcatcgaacggctggcgaaagcggccggtccggaagggatggtcgccggtcaggcagccgatatggaaggagaggggaaaacgctgacgctttcggagctcgaatacattcatcggcataaaaccgggaaaatgctgcaatacagcgtgcacgccggcgccttgatcggcggcgctgatgcccggcaaacgcgggagcttgacgaattcgccgcccatctaggccttgcctttcaaattcgcgatgatattctcgatattgaaggggcagaagaaaaaatcggcaagccggtcggcagcgaccaaagcaacaacaaagcgacgtatccagcgttgctgtcgcttgccggcgcgaaggaaaagttggcgttccatatcgaggcggcgcagcgccatttacggaacgctgacgttgacggcgccgcgctcgcctatatttgcgaactggtcgccgcccgcgaccattaa

SEQ ID NO: 20 - DNA, 573 bp, Escherichia coli
  CDS (1)..(573)
atgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaactcgagcaccaccaccaccaccactga

SEQ ID NO: 21 - protein, 190 aa, Escherichia coli
MQTEHVILLNAQGVPTGTLEKYAAHTADTRLHLAFSSWLFNAKGQLLVTRRALSKKAWPGVWTNSVCGHPQLGESNEDAVIRRCRYELGVEITPPESIYPDFRYRATDPSGIVENEVCPVFAARTTSALQINDDEVMDYQWCDLADVLHGIDATPWAFSPWMVMQATNREARKRLSAFTQLKLEHHHHHH

SEQ ID NO: 22 - DNA, 543 bp, Artificial Sequence
  EcIDIMS4
  CDS (1)..(543)
atgcagacagaacacgtgattttgttgaacgcacaaggcgttcctattgggacattagaaaagtacgccgcgcataccgcagatacacgccttcacttggcgttctcgtcctggctgttcaacgcgaaaggacaactgctggtaacacgtcgcgcattaagtaaaaaggcttggcctggggtttggactaacagcgtatgcggccatccgcagttgggagagtcaaatgaggacgcagtaatccgtcgctgccgctatgaactgggagttgaaattacacctcctgaacctatctacccagatttccgctaccgcgcaacagacccgtctggaattgtggaaaatgaagtttgccctgtcttcgctgcccgcacaacctccgccttacagattaacgatgacgaggtgatggactatcagtggtgtgatcttgcagatgtgttacacgggattgacgccacaccctgggccttttccccgtggatggtgatgcaggcaaccaacgaggaggctcgtaaacgtctgcaggcattcacgcaataa

SEQ ID NO: 23 - protein, 180 aa, Artificial Sequence
  Synthetic Construct
MQTEHVILLNAQGVPIGTLEKYAAHTADTRLHLAFSSWLFNAKGQLLVTRRALSKKAWPGVWTNSVCGHPQLGESNEDAVIRRCRYELGVEITPPEPIYPDFRYRATDPSGIVENEVCPVFAARTTSALQINDDEVMDYQWCDLADVLHGIDATPWAFSPWMVMQATNEEARKRLQAFTQ

SEQ ID NO: 24 - DNA, 543 bp, Artificial Sequence
  EcIDIM10
  CDS (1)..(543)
atgcaaaccgagcatgtcattttattggacgagcaaggagaaccaattggaactttagaaaaatacgctgcacatacagcggacacccgcttacatcttgctttttctagttggctgtttaacgataagggtcaattattagtgacgcgccgtgcgctgagcaaaaaagcatggccgggtgtttggacgaacagtgtttgcggacacccccaactgggagaatccaatgaggatgcggtaattcgccgttgtcgctatgaattgggtgtggagattacgccaccgacaccgatctaccctgatttccgttatcgcgctacggatccttcaggtattgttgaaaatgaagtatgcccagtgtttgccgcgcgcacaacttctgcgcttcaaatcaacccagacgaggtcatggattaccaatggtgtgatcttgctgacgtactgcacgggattgacgcgacaccgtgggcttttagtccctggatggttatgcaagcgacaaatgaagaagcacgtaagcgccttcaggcgtttactcagtaa

SEQ ID NO: 25 - protein, 180 aa, Artificial Sequence
  Synthetic Construct
MQTEHVILLDEQGEPIGTLEKYAAHTADTRLHLAFSSWLFNDKGQLLVTRRALSKKAWPGVWTNSVCGHPQLGESNEDAVIRRCRYELGVEITPPTPIYPDFRYRATDPSGIVENEVCPVFAARTTSALQINPDEVMDYQWCDLADVLHGIDATPWAFSPWMVMQATNEEARKRLQAFTQ

SEQ ID NO: 26 - DNA, 543 bp, Artificial Sequence
  EcIDIM17
  CDS (1)..(543)
atgcagaccgagcatgtaatccttcttgatgagcaggggaatccaattggtacattaccaaagtacgctgcccatacggcggacactcgcttacatttggcgttctcatcctggttgtttaacgacaaaggacaattgctggtaacgcgccgcgccctgtctaagaaggcgtggcctggggtatggaccaactccgtctgcggacaccctcaattaggtgaatcgctggaggatgcagtccgccgccgcgcccgctacgaacttggtgtggaaattacgcccccaacccctatctacccggattttcgctatcgtgcaacagacccaagtgggatcgtagagaatgaggtttgtcccgtatttgcagcgcgtaccacgtccgcattacagatcaatccagacgaagtcatggactggcaatggtgcgatcttgcggatgttttgcatgggatcgacgctactccctgggcctttagcccatggatggttatgcaagcggcccatgaggaggcccgtaaacgccttcaagcctttactcaataa

SEQ ID NO: 27 - protein, 180 aa, Artificial Sequence
  Synthetic Construct
MQTEHVILLDEQGNPIGTLPKYAAHTADTRLHLAFSSWLFNDKGQLLVTRRALSKKAWPGVWTNSVCGHPQLGESLEDAVRRRARYELGVEITPPTPIYPDFRYRATDPSGIVENEVCPVFAARTTSALQINPDEVMDWQWCDLADVLHGIDATPWAFSPWMVMQAAHEEARKRLQAFTQ

SEQ ID NO: 28 - DNA, 50 bp, Artificial Sequence
  Forward Primer EcThiM
ccgcgcggcagccatatgcaagtcgacctgctgggttcagcgcaatctgc

SEQ ID NO: 29 - DNA, 58 bp, Artificial Sequence
  Reverse Primer EcThiM
ggtggtggtggtggtgctcgagttatgcctgcacctcctgcgtcaattgccagagcgc

SEQ ID NO: 30 - protein, 307 aa, Streptomyces sp. [sequence omitted]

SEQ ID NO: 31 - DNA, 46 bp, Artificial Sequence
  Forward Primer RpMatB
ccgcgcggcagccatatgaacgccaacctgttcgcccgcctgttcg

SEQ ID NO: 32 - DNA, 56 bp, Artificial Sequence
  Reverse Primer RpMatB
ggtggtggtggtggtgctcgagttacttgtagatgtccttgtaggtctcgcgcagg

SEQ ID NO: 33 - DNA, 45 bp, Artificial Sequence
  Forward Primer GtADK
ggtgccgcgcggcagccatatgaatttagtgctgatggggctgcc

SEQ ID NO: 34 - DNA, 48 bp, Artificial Sequence
  Reverse Primer GtADK
cagtggtggtggtggtggtgctcgagttatcgagtaagtcccccgagc

SEQ ID NO: 35 - DNA, 984 bp, Artificial Sequence
  NphBM31s
  CDS (1)..(984)
atgggcagcagccatcatcatcatcatcacagcagcggcctggtgccgcgcggcagccatatgtcggaagctgccgatgtagaacgtgtctacgccgccatcgaagaagccgcaggtttgttgggggtcgcatgcgcacgcgataagatttggcccttgctgtcaacattccaggataccttggttgagggtggaagcgtagttgtttttagcatggcctcggggcgtcactcaacggagctggacttctcaatttccgtcccgcctagtcatggcgatccgtacgcgattgtggtggaaaagggcttgttcccggcaactggacatccagttgatgaccttctggcggacattcagaagcatcttcccgtatctatgtttgcgattgacggggaagttaccggggggttcaaaaaaacttatgcgttcttcccgaccgataacatgcccggtgtcgcggaactggcggccatcccatcgatgcctcctgcagtcgctgaaaatgctgaactgttcgcgcgttatggcctggacaaggtacaaatgacctcgatggattataaaaaacgtcaagtgaacctgtatttctccgaactgtcggctcagacgctggaggctgaatcagtacttgctttagtgcgtgaactgggtcttcatgtcccaaacgagctgggtctgaaattttgcaaacgctccttctcagtatacccaacattaaactgggacacctcgaagattgaccgcctttgcttctctgtaatcagtacagatccgacacttgtacctagctcagacgagggagacattgaaaaatttcacaattacgctacaaaggccccctatgcatatgttggagaaaagcgtacacttgtttacggcttgactttatctcccaaagaggagtattataaattgggtgccgtttaccacattactgacgtacaacgcaaacttttgaaggcgttcgacagccttgaggattaa

SEQ ID NO: 36 - protein, 327 aa, Artificial Sequence
  Synthetic Construct
MGSSHHHHHHSSGLVPRGSHMSEAADVERVYAAIEEAAGLLGVACARDKIWPLLSTFQDTLVEGGSVVVFSMASGRHSTELDFSISVPPSHGDPYAIVVEKGLFPATGHPVDDLLADIQKHLPVSMFAIDGEVTGGFKKTYAFFPTDNMPGVAELAAIPSMPPAVAENAELFARYGLDKVQMTSMDYKKRQVNLYFSELSAQTLEAESVLALVRELGLHVPNELGLKFCKRSFSVYPTLNWDTSKIDRLCFSVISTDPTLVPSSDEGDIEKFHNYATKAPYAYVGEKRTLVYGLTLSPKEEYYKLGAVYHITDVQRKLLKAFDSLED

SEQ ID NO: 37 - protein, 307 aa, Artificial Sequence
  NphB-M31 (Y288V/A232S)
MSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFSVISNDPTLVPSSDEGDIEKFHNYATKAPYAYVGEKRTLVYGLTLSPKEEYYKLGAVYHITDVQRGLLKAFDSLED

SEQ ID NO: 38 - protein, 307 aa, Artificial Sequence
  NphB-M31S2 is NphB-M31 (Y288V/A232S)  + M14I, Y31W, T69P, T77I, E80A, D93S, T98I, T126P, M129L, G131Q, S136A, E222D, G224S, N236T, S277T, and G297K
MSEAADVERVYAAIEEAAGLLGVACARDKIWPLLSTFQDTLVEGGSVVVFSMASGRHSTELDFSISVPPSHGDPYAIVVAKGLFPATGHPVDSLLADIQKHLPVSMFAIDGEVTGGFKKTYAFFPPDNLPQVAELAAIPSMPPAVAENAELFARYGLDKVQMTSMDYKKRQVNLYFSELSAQTLEAESVLALVRELGLHVPNELGLKFCKRSFSVYPTLNWDTSKIDRLCFSVISTDPTLVPSSDEGDIEKFHNYATKAPYAYVGEKRTLVYGLTLTPKEEYYKLGAVYHITDVQRKLLKAFDSLED

SEQ ID NO: 39 - protein, 307 aa, Artificial Sequence
  NphB-M31S3 is NphB-M31 (Y288V/A232S)  + M14I, L33I, Y31W, T69P, T77I, V78A, E80A, D93S, T98I, E112G, T114V, T126P, M129L, G131Q, S136A, E222D, G224S, K225Q, N236T, S277T, and G297K
MSEAADVERVYAAIEEAAGLLGVACARDKIWPILSTFQDTLVEGGSVVVFSMASGRHSTELDFSISVPPSHGDPYAIAVAKGLFPATGHPVDSLLADIQKHLPVSMFAIDGGVVGGFKKTYAFFPPDNLPQVAELAAIPSMPPAVAENAELFARYGLDKVQMTSMDYKKRQVNLYFSELSAQTLEAESVLALVRELGLHVPNELGLKFCKRSFSVYPTLNWDTSQIDRLCFSVISTDPTLVPSSDEGDIEKFHNYATKAPYAYVGEKRTLVYGLTLTPKEEYYKLGAVYHITDVQRKLLKAFDSLED

SEQ ID NO: 40-53 - skipped (14 entries)

SEQ ID NO: 54 - DNA, 789 bp, Escherichia coli
  CDS (1)..(789)
atgcaagtcgacctgctgggttcagcgcaatctgcgcacgcgttacacctttttcaccaacattcccctcttgtgcactgcatgaccaatgatgtggtgcaaacctttaccgccaataccttgctggcgctcggtgcatcgccagcgatggttatcgaaaccgaagaggccagtcagtttgcggctatcgccagtgccttgttgattaacgttggcacactgacgcagccacgcgctcaggcgatgcgtgctgccgttgagcaagcaaaaagctctcaaacaccctggacgcttgatccagtagcggtgggtgcgctcgattatcgccgccatttttgtcatgaacttttatcttttaaaccggcagcgatacgtggtaatgcttcggaaatcatggcattagctggcattgctaatggcggacggggagtggataccactgacgccgcagctaacgcgatacccgctgcacaaacactggcacgggaaactggcgcaatcgtcgtggtcactggcgagatggattatgttaccgatggacatcgtatcattggtattcacggtggtgatccgttaatgaccaaagtggtaggaactggctgtgcattatcggcggttgtcgctgcctgctgtgcgttaccaggcgatacgctggaaaatgtcgcatctgcctgtcactggatgaaacaagccggagaacgcgcagtcgccagaagcgaggggccaggcagttttgttccacatttccttgatgcgctctggcaattgacgcaggaggtgcaggcatga

SEQ ID NO: 55 - protein, 262 aa, Escherichia coli; identical to SEQ ID NO: 2

SEQ ID NO: 56 - DNA, 819 bp, Bacillus subtilis
  CDS (1)..(819)
atggatgcacaatcagcagcaaaatgtcttacggctgtccgccggcatagcccactggtgcatagcataaccaacaatgtcgtaacgaatttcacagcaaacggcctgctcgcgctcggcgcatcgcccgttatggcgtacgcaaaagaagaggtcgccgatatggcgaaaattgcgggtgcactcgttttaaatatcggaacactgagcaaggagtcagtcgaagcgatgatcatcgcgggaaaatcagctaatgaacatggcgttcccgtcattcttgatcctgtcggtgccggagcaacaccgttccgcactgaatcggcacgtgacatcattcgtgaggtgcgccttgctgcaatcagaggaaatgcggcggaaattgcccataccgtcggcgtgaccgattggctgatcaaaggtgttgatgcgggtgaaggtggaggcgacatcatccggctggctcagcaggcggcacaaaagctaaacacggtcattgcgataactggtgaagttgatgtcatagccgacacgtcacatgtatacacccttcataacggccacaagctgctgacaaaagtgacaggcgccggttgcctgctgacttccgtcgtcggtgcgttttgcgctgtggaagaaaatccattgtttgctgctattgcggccatttcttcgtatggggtcgccgctcagcttgccgcacagcagacggctgacaaaggccctggaagctttcagattgaattgctgaacaagctttcaactgttactgaacaagacgtccaagaatgggcgactatagaaagggtgactgtctcatga

SEQ ID NO: 57 - protein, 272 aa, Bacillus subtilis
MDAQSAAKCLTAVRRHSPLVHSITNNVVTNFTANGLLALGASPVMAYAKEEVADMAKIAGALVLNIGTLSKESVEAMIIAGKSANEHGVPVILDPVGAGATPFRTESARDIIREVRLAAIRGNAAEIAHTVGVTDWLIKGVDAGEGGGDIIRLAQQAAQKLNTVIAITGEVDVIADTSHVYTLHNGHKLLTKVTGAGCLLTSVVGAFCAVEENPLFAAIAAISSYGVAAQLAAQQTADKGPGSFQIELLNKLSTVTEQDVQEWATIERVTVS

SEQ ID NO: 58 - DNA, 783 bp, Methanocaldococcus jannaschii
  CDS (1)..(783)
atgttgactattcttaagttgggagggagcattctgtccgataaaaacgttccatatagcattaagtgggataacttagaacgtattgctatggaaatcaaaaacgcgttagattattacaagaaccaaaataaagaaattaagcttattctggtacatggcggcggggcatttgggcatccagtggccaagaaatacctgaagattgaagacggcaaaaaaattttcatcaacatggaaaaaggattctgggagattcagcgtgcgatgcgccgttttaataacatcatcatcgacacgcttcagagttacgatatcccagcggtctcgattcaaccttccagctttgttgtttttggcgacaaattgatcttcgacacctctgcgatcaaagagatgttgaaacgcaaccttgtacccgttatccatggggatatcgtcattgacgataaaaatgggtaccgtattatcagcggtgacgacatcgtgccatatttagccaatgaactgaaggcagatttaatcctttatgcaaccgacgtggacggcgtattgattgacaacaagcccattaaacgcattgataagaataatatctacaagattttgaattatctttcgggtagcaattcaattgacgtcacgggggggatgaaatacaagatcgacatgatccgtaaaaacaaatgccgtggtttcgtgtttaatggcaacaaggcaaacaacatttataaggcgctgcttggggaagtcgagggtaccgaaatcgacttttctgaataa

SEQ ID NO: 59 - protein, 260 aa, Methanocaldococcus jannaschii
MLTILKLGGSILSDKNVPYSIKWDNLERIAMEIKNALDYYKNQNKEIKLILVHGGGAFGHPVAKKYLKIEDGKKIFINMEKGFWEIQRAMRRFNNIIIDTLQSYDIPAVSIQPSSFVVFGDKLIFDTSAIKEMLKRNLVPVIHGDIVIDDKNGYRIISGDDIVPYLANELKADLILYATDVDGVLIDNKPIKRIDKNNIYKILNYLSGSNSIDVTGGMKYKIDMIRKNKCRGFVFNGNKANNIYKALLGEVEGTEIDFSE

SEQ ID NO: 60 - DNA, 744 bp, Methanothrix themoacetophila
  CDS (1)..(744)
ttaaagattttgaaattgggcggtagcattattacggataagagccgcttagctactgcacgtctggatcaaatttcacgtatcgcacacgaaatctcaggcatcgagaacctgattgttgttcacggagccggttcttttggtcacatccatgccaaaaatttcggtcttccggaacgtttctcaggagaagggttactgaaaacacatctgtcggtctcggatttgaatcgtatcgtcgttgaagctcttcatgatgcaggggtggacgcgctgcccttgcaccccttatcaagtgtagtccttcgtgacggacgcatccaccatatgtctaccgaggtcattacggaaatgcttcgtcgtgatgtagtgccggtattacatggggatgttgcgatggacctgtcaaagggtgccggcattgtaagtggagaccagttggtttcgtatatggcacgtactctgggagctggtatggtcgctatggggaccgatgtcgacggggttatgatcgatggtcgtgtccttagttgcattacacctaatgacatgcactctttggagagtcacttattacccgcaaaaggggtagacgtcacgggtggaatgcgcggtaaactggcggaattagtagagctggcaggcattggaattgattcgcgtatttttaatgccggcgttgctggtaatgtacgccgtgctttgtctggggagtcgttaggaactttgattactggacgctaa

SEQ ID NO: 61 - protein, 247 aa, Methanothrix themoacetophila
LKILKLGGSIITDKSRLATARLDQISRIAHEISGIENLIVVHGAGSFGHIHAKNFGLPERFSGEGLLKTHLSVSDLNRIVVEALHDAGVDALPLHPLSSVVLRDGRIHHMSTEVITEMLRRDVVPVLHGDVAMDLSKGAGIVSGDQLVSYMARTLGAGMVAMGTDVDGVMIDGRVLSCITPNDMHSLESHLLPAKGVDVTGGMRGKLAELVELAGIGIDSRIFNAGVAGNVRRALSGESLGTLITGR

SEQ ID NO: 62 - DNA, 543 bp, Escherichia coli; identical to SEQ ID NO: 24
  CDS (1)..(543)

SEQ ID NO: 63 - protein, 180 aa, Escherichia coli; identical to SEQ ID NO: 25

SEQ ID NO: 64 - DNA, 894 bp, Geobacillus stearothermophilus
  CDS (1)..(894)
atggcgcagctttcagttgaacagtttctcaacgagcaaaaacaggcggtggaaacagcgctctcccgttatatagagcgcttagaagggccggcgaagctgaaaaaggcgatggcgtactcattggaggccggcggcaaacgaatccgtccgttgctgcttctgtccaccgttcgggcgctcggcaaagacccggcggtcggattgcccgtcgcctgcgcgattgaaatgatccatacgtactttttgatccatgatgatttgccgagcatggacaacgatgatttgcggcgcggcaagccgacgaaccataaagtgttcggcgaggcgatggccatcttggcgggggacgggttgttgacgtacgcgtttcaattgatcaccgaaatcgacgatgagcgcatccctccttccgtccggcttcggctcatcgaacggctggcgaaagcggccggtccggaagggatggtcgccggtcaggcagccgatatggaaggagaggggaaaacgctgacgctttcggagctcgaatacattcatcggcataaaaccgggaaaatgctgcaatacagcgtgcacgccggcgccttgatcggcggcgctgatgcccggcaaacgcgggagcttgacgaattcgccgcccatctaggccttgcctttcaaattcgcgatgatattctcgatattgaaggggcagaagaaaaaatcggcaagccggtcggcagcgaccaaagcaacaacaaagcgacgtatccagcgttgctgtcgcttgccggcgcgaaggaaaagttggcgttccatatcgaggcggcgcagcgccatttacggaacgctgacgttgacggcgccgcgctcgcctatatttgcgaactggtcgccgcccgcgaccattaa

SEQ ID NO: 65 - protein, 297 aa, Geobacillus stearothermophilus
MAQLSVEQFLNEQKQAVETALSRYIERLEGPAKLKKAMAYSLEAGGKRIRPLLLLSTVRALGKDPAVGLPVACAIEMIHTYFLIHDDLPSMDNDDLRRGKPTNHKVFGEAMAILAGDGLLTYAFQLITEIDDERIPPSVRLRLIERLAKAAGPEGMVAGQAADMEGEGKTLTLSELEYIHRHKTGKMLQYSVHAGALIGGADARQTRELDEFAAHLGLAFQIRDDILDIEGAEEKIGKPVGSDQSNNKATYPALLSLAGAKEKLAFHIEAAQRHLRNADVDGAALAYICELVAARDH

SEQ ID NO: 66 - DNA, 1632 bp, Cannabis sativa
  CDS (1)..(1632)
atggaaaagagtggctacggacgcgacggtatttaccgtagcctgcgtcctcctttacacctgccaaacaataacaatttgagtatggtctcattcctgttccgtaacagcagcagctatccacagaaaccggcgttgatcgatagcgagactaatcaaattttatcttttagtcattttaaaagcaccgtgatcaaggtctcccatggcttcttaaacctggggatcaaaaagaatgacgtggttttaatctacgcacccaattcgatccactttcccgtatgcttccttggcattattgcttctggggcgatcgccactacttcaaatccattatacaccgtgagtgagttgtcgaaacaagtaaaggactcgaaccctaaattgattatcacagtccctcagttattggaaaaggtcaagggtttcaatctgccaactatccttatcggccctgattctgagcaggaatcgtctagtgataaagtaatgactttcaatgatctggtcaatctgggaggaagttcgggtagcgaattccctatcgtcgacgatttcaagcaatccgacaccgccgcactgttgtactcaagtggcacgacaggtatgagcaagggggtcgttctgacgcacaaaaattttattgcctcatcgttgatggtaacaatggaacaggacttggtcggcgagatggacaatgtgttcctgtgtttccttcctatgtttcacgtctttggcttagccattattacgtatgctcagttacagcgcggtaataccgtgatttcaatggcccgctttgacttggaaaagatgttaaaagatgttgaaaagtacaaagttacccacctttgggtcgtacccccagttatcttagcgttgtcgaagaactcaatggtgaaaaaattcaatttgtcatccatcaagtatattggttcaggcgctgcgccattaggaaaggatctgatggaagaatgctctaaggtggttccttacggaatcgtggctcaaggatatggcatgacggaaacgtgcggaatcgtatccatggaagacatccgcggcgggaaacgcaattcagggtcggccggaatgttggcaagtggggtagaagctcagatcgtgagtgtggacaccttaaaaccccttcccccgaatcaattaggggaaatctgggtaaaaggtccaaatatgatgcaaggctatttcaacaatcctcaagcgaccaaacttaccattgataaaaagggttgggttcatactggcgacttggggtatttcgacgaagacggacacttatatgttgtagaccgtattaaggagcttattaaatacaagggattccaagttgcgcctgcggaactggagggattattagttagtcaccccgagatcttagacgcggtagttattcccttccccgatgctgaggcaggcgaagtcccggtggcatacgttgttcgctcgcctaacagttcgttgaccgaaaatgacgttaaaaaattcatcgccggtcaggtcgcctcctttaagcgtctgcgcaaggttacttttattaattccgtccccaagagcgcaagtgggaagattctgcgccgcgagcttattcaaaaggttcgctctaacatgtaa

SEQ ID NO: 67 - protein, 543 aa, Cannabis sativa
MEKSGYGRDGIYRSLRPPLHLPNNNNLSMVSFLFRNSSSYPQKPALIDSETNQILSFSHFKSTVIKVSHGFLNLGIKKNDVVLIYAPNSIHFPVCFLGIIASGAIATTSNPLYTVSELSKQVKDSNPKLIITVPQLLEKVKGFNLPTILIGPDSEQESSSDKVMTFNDLVNLGGSSGSEFPIVDDFKQSDTAALLYSSGTTGMSKGVVLTHKNFIASSLMVTMEQDLVGEMDNVFLCFLPMFHVFGLAIITYAQLQRGNTVISMARFDLEKMLKDVEKYKVTHLWVVPPVILALSKNSMVKKFNLSSIKYIGSGAAPLGKDLMEECSKVVPYGIVAQGYGMTETCGIVSMEDIRGGKRNSGSAGMLASGVEAQIVSVDTLKPLPPNQLGEIWVKGPNMMQGYFNNPQATKLTIDKKGWVHTGDLGYFDEDGHLYVVDRIKELIKYKGFQVAPAELEGLLVSHPEILDAVVIPFPDAEAGEVPVAYVVRSPNSSLTENDVKKFIAGQVASFKRLRKVTFINSVPKSASGKILRRELIQKVRSNM

SEQ ID NO: 68 - DNA, 2163 bp, Cannabis sativa
  CDS (1)..(2163)
atgggtaagaattacaagtccctggactctgttgtggcctctgacttcatagccctaggtatcacctctgaagttgctgagacactccatggtagactggccgagatcgtgtgtaattatggcgctgccactccccaaacatggatcaatattgccaaccatattctgtcgcctgacctccccttctccctgcaccagatgctcttctatggttgctataaagactttggacctgcccctcctgcttggatacccgacccggagaaagtaaagtccaccaatctgggcgcacttttggagaagcgaggaaaagagtttttgggagtcaagtataaggatcccatttcaagcttttctcatttccaagaattttctgtaagaaaccctgaggtgtattggagaacagtactaatggatgagatgaagataagtttttcaaaggatccagaatgtatattgcgtagagatgatattaataatccagggggtagtgaatggcttccaggaggttatcttaactcagcaaagaattgcttgaatgtaaatagtaacaagaaattgaatgatacaatgattgtatggcgtgatgaaggaaatgatgatttgcctctaaacaaattgacacttgaccaattgcgtaaacgtgtttggttagttggttatgcacttgaagaaatgggtttggagaagggttgtgcaattgcaattgatatgccaatgcatgtggatgctgtggttatctatctagctattgttcttgcgggatatgtagttgtttctattgctgatagtttttctgctcctgaaatatcaacaagacttcgactatcaaaagcaaaagccatttttacacaggatcatattattcgtgggaagaagcgtattcccttatacagtagagttgtggaagccaagtctcccatggccattgttattccttgtagtggctctaatattggtgcagaattgcgtgatggcgatatttcttgggattactttctagaaagagcaaaagagtttaaaaattgtgaatttactgctagagaacaaccagttgatgcctatacaaacatcctcttctcatctggaacaacaggggagccaaaggcaattccatggactcaagcaactcctttaaaagcagctgcagatgggtggagccatttggacattaggaaaggtgatgtcattgtttggcccactaatcttggttggatgatgggtccttggctggtctatgcttcactccttaatggggcttctattgccttgtataatggatcaccacttgtttctggctttgccaaatttgtgcaggatgctaaagtaacaatgctaggtgtggtccctagtattgttcgatcatggaaaagtaccaattgtgttagtggctatgattggtccaccatccgttgcttttcctcttctggtgaagcatctaatgtagatgaatacctatggttgatggggagagcaaactacaagcctgttatcgaaatgtgtggtggcacagaaattggtggtgcattttctgctggctctttcttacaagctcaatcattatcttcatttagttcacaatgtatgggttgcactttatacatacttgacaagaatggttatccaatgcctaaaaacaaaccaggaattggtgaattagcgcttggtccagtcatgtttggagcatcgaagactctgttgaatggtaatcaccatgatgtttattttaagggaatgcctacattgaatggagaggttttaaggaggcatggggacatttttgagcttacatctaatggttattatcatgcacatggtcgtgcagatgatacaatgaatattggaggcatcaagattagttccatagagattgaacgagtttgtaatgaagttgatgacagagttttcgagacaactgctattggagtgccacctttgggcggtggacctgagcaattagtaattttctttgtattaaaagattcaaatgatacaactattgacttaaatcaattgaggttatctttcaacttgggtttacagaagaaactaaatcctctgttcaaggtcactcgtgttgtgcctctttcatcacttccgagaacagcaaccaacaagatcatgagaagggttttgcgccagcaattttctcactttgaatga

SEQ ID NO: 69 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 70 - DNA, 1695 bp, Cannabis sativa
  CDS (1)..(1695)
atggaagtactgaaggaggttgcgaaggaaggtagcgcagcccgtgaaggtgtcgctattcgcgccgaccagaaatcgtacagctataagcaattgatctcctccgcgcagtcgatctgctcactgttatgcggtactgaacttaaagcgattcacgaagccgggaaacaagctcgtcctagcgcgtctatcaatggggccgggggtcacggccacttgggaggagctcgtattggaattgttgctaagccgtcggcagaatttgtagccggtgttttaggtacgtggttatctggtggagttgcggttccccttgcactgtcttacccggaggcggaattactgcatgtcatgaacgattcagatatcagcatgatcttgagcacggaagaccatcaagaactgatgcaaaatattgctgccaagacttccgcacagttttccttaattccatctgtgccgtcgtcgtgctcacaagaagtagcggtcgatcatcgtcagaccggtgacatctctaccgactctatcttgcttaaccgcgagatctctaacgagaatcccgcacttatcgtctatacgtcggggacgacaggcaagccgaagggcgtcgttcacacacaccaatcaatttctgcacaggttcagacgttagccaaggcatgggagtatactcctgccgatcaattcttacactgcttaccgctgcatcatgtgcatgggctgtttaacgcactgttcgcgcccctttacgcgcgttcaacagttgaatttctgccgaaattttctgtccgcggtatttggcaacgctggcgcgaatcctacccaacgtcagagacgaaagccaatgactgcattacggtatttacaggagttcccaccatgtacacgcgtctgattcaaggatatgaagctatggatccagagttaaaagaggcctctgcatctgctgctaagcagctgcgccttatgatgtgtggttcctctgcgctgccagttcctgtcatgcagcagtggcaaaccatcaccggccaccgtcttctggaacgttacggaatgaccgaatttgtcatggcaatttctaaccccttgaaaggtgagcgcaaatccggtactgtcggaaagccgtttccaggtgtagaggtgcgcattttagcagaggatgaaaacggcgatgatgctaccggggtgggagagctgtgcgtacgcagtccgtcccttttcaaagagtattggcgtttgcccgaggtcacaaaagcctcctttacagacgacggctttttcaaaaccggcgacgcaggcaaggtcgatgaggacggttactacgtgattctgggccgtactagcgcagatattatgaaagttggaggctataagctgtctgctctggaaatcgagtcggtccttctggaacacccgactgtcgaggaatgctgtgtcttgggacttcccgacaaggattatggggaagccgtatccgcaatcattgtaccggcagccgaggcgaagaagaaacgcgaagaggagtcacgccccgccattagtctggaggaactgttctcatgggcacagcacaaacttgccccctacaaactgcccacgcgtttattcctgtgggactctttacctcgcaacgcaatggggaaagtcaacaaaaaagagctgaagaaaaaactgacagttgagcaaggtatttaa

SEQ ID NO: 71 - protein, 564 aa, Cannabis sativa
MEVLKEVAKEGSAAREGVAIRADQKSYSYKQLISSAQSICSLLCGTELKAIHEAGKQARPSASINGAGGHGHLGGARIGIVAKPSAEFVAGVLGTWLSGGVAVPLALSYPEAELLHVMNDSDISMILSTEDHQELMQNIAAKTSAQFSLIPSVPSSCSQEVAVDHRQTGDISTDSILLNREISNENPALIVYTSGTTGKPKGVVHTHQSISAQVQTLAKAWEYTPADQFLHCLPLHHVHGLFNALFAPLYARSTVEFLPKFSVRGIWQRWRESYPTSETKANDCITVFTGVPTMYTRLIQGYEAMDPELKEASASAAKQLRLMMCGSSALPVPVMQQWQTITGHRLLERYGMTEFVMAISNPLKGERKSGTVGKPFPGVEVRILAEDENGDDATGVGELCVRSPSLFKEYWRLPEVTKASFTDDGFFKTGDAGKVDEDGYYVILGRTSADIMKVGGYKLSALEIESVLLEHPTVEECCVLGLPDKDYGEAVSAIIVPAAEAKKKREEESRPAISLEELFSWAQHKLAPYKLPTRLFLWDSLPRNAMGKVNKKELKKKLTVEQGI

SEQ ID NO: 72 - DNA, 1158 bp, Cannabis sativa
  CDS (1)..(1158)
atgaatcatctgcgtgctgaaggaccagcttccgtattggcaattggaacagctaaccctgagaacattcttcttcaggatgagtttcccgactattacttccgcgtgacaaagagcgaacacatgacacagcttaaagagaagttccgtaagatctgtgacaaaagcatgatccgcaaacgtaactgcttccttaacgaggagcatctgaagcagaatccccgtcttgttgaacatgagatgcagaccttggatgctcgccaggacatgttggttgttgaggtccctaagctgggcaaagatgcgtgtgcaaaagcgattaaagagtgggggcagcctaaaagcaaaattactcatctgattttcacaagcgccagtacaaccgatatgcccggtgcggactaccattgtgcaaaattattgggtttatcgccttcagtaaaacgtgttatgatgtaccagttaggatgctacggtggtggcaccgtacttcgtattgcgaaggacatcgccgagaacaacaaaggagcccgtgtacttgctgtatgttgtgatatcatggcgtgcctttttcgcggccccagcgagagtgaccttgagttacttgtggggcaggccatcttcggagacggtgccgcagccgtcattgttggcgcagagcccgatgaatccgttggcgagcgcccgatctttgagcttgtaagtacaggacaaactatcttgcccaactctgaggggactatcggcggacatattcgtgaggcgggcttgatttttgaccttcacaaggatgttccaatgcttatctccaataatattgaaaaatgtcttatcgaagcattcactccgattggtatctccgattggaattcgattttttggatcacccatcctggtgggaaagctattttagacaaggtggaggagaaattacatcttaagtcagataagtttgtcgacagtcgccacgtgttgtcggaacatggcaacatgtcatcgtcaaccgtcttgttcgttatggacgaattacgtaaacgcagtttagaagagggtaagagtacgacgggggacgggttcgagtggggagtcttattcgggttcggtccaggattgacagtggaacgcgtcgtggttcgcagtgtccccattaagtactaa

SEQ ID NO: 73 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 74 - DNA, 306 bp, Cannabis sativa
  CDS (1)..(306)
atggcagtcaaacacttgatcgtgttaaagttcaaagatgaaatcacagaggctcagaaggaagaatttttcaagacgtatgtaaaccttgttaatatcatccccgctatgaaggatgtgtattggggtaaagacgtgacacagaagaacaaagaggaaggctacacgcacatcgtagaggtcacatttgagagcgtcgaaactattcaggattacatcattcatcccgcacacgttggattcggggatgtgtatcgctctttctgggaaaaattgctgatcttcgactatacaccgcgtaagtaa

SEQ ID NO: 75 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK